(19) United States
(12) Patent Application Publication
(10) Pub. No.: US 2026/0035694 A1
(43) Pub. Date: Feb. 5, 2026

(54) ANTISENSE OLIGOMERS FOR TREATMENT OF NON-SENSE MEDIATED RNA DECAY BASED CONDITIONS AND DISEASES

(71) Applicant: Stoke Therapeutics, Inc., Bedford, MA (US)

(72) Inventors: Isabel Aznarez, Boston, MA (US); Jacob Kach, Jamaica Plain, MA (US); Mikaela Downs, Lowell, MA (US); Sebastien Matthieu Hugues Weyn-Vanhentenryck, Cambridge, MA (US); Ana Corrionero Saiz, Cambridge, MA (US)

(21) Appl. No.: 18/869,417

(22) PCT Filed: Jun. 1, 2023
(86) PCT No.: PCT/US2023/024182

§ 371 Date: Nov. 26, 2024

Related U.S. Application Data
(60) Provisional application No. 63/347,743, filed on Jun. 1, 2022.

(51) Int. Cl. C12N 15/113

(52) U.S. Cl. CPC C12N 15/113; C12N 2310/11; C12N 2320/33

(57) ABSTRACT

Alternative splicing events in genes can lead to non-productive mRNA transcripts which in turn can affect protein expression level, and therapeutic agents which can target the alternative splicing events in genes can modulate the expression level of functional proteins in patients and/or inhibit aberrant protein expression. Such therapeutic agents can be used to treat a condition or disease caused by protein deficiency.

### CROSS-REFERENCE

This application is a national phase entry of International Application No. PCT/US2023/024182, filed Jun. 1, 2023, which claims the benefit of U.S. Provisional Application No. 63/347,743, filed Jun. 1, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jul. 13, 2023, is named 47991-733_831_SL.xml and is 1,482,762 bytes in size.

### BACKGROUND

Alternative splicing events in genes can lead to non-productive mRNA transcripts which in turn can affect protein expression levels, and therapeutic agents which can target the alternative splicing events in genes can modulate the expression level of functional proteins in patients. Such therapeutic agents can be used to treat a condition or disease caused by protein deficiency.

### SUMMARY

Described herein, in certain embodiments, is a method of modulating expression of a target protein in a cell having a pre-mRNA that is transcribed from a target gene and that comprises a non-sense mediated RNA decay-inducing exon (NMD exon), the method comprising contacting an agent or a vector encoding the agent to the cell, whereby the agent modulates splicing of the NMD exon from the pre-mRNA, thereby modulating the level of a processed mRNA that is processed from the pre-mRNA, and modulating the expression of the target protein in the cell, wherein the target gene is a SETD5 gene. Described herein, in certain embodiments, is a method of treating or reducing the likelihood of developing a disease or condition in a subject in need thereof by modulating expression of a target protein in a cell of the subject, comprising: contacting an agent or a vector encoding the agent to the cell of the subject, whereby the agent modulates splicing of a non-sense mediated RNA decay-inducing exon (NMD exon) from a pre-mRNA that is transcribed from a target gene and that comprises the NMD exon, thereby modulating the level of a processed mRNA that is processed from the pre-mRNA, and modulating the expression of the target protein in the cell of the subject, wherein the target gene is a SETD5 gene.

Described herein, in certain embodiments, is a method of modulating expression of a target protein in a cell having a pre-mRNA that is transcribed from a target gene and that comprises a first exon that comprises a translational start site, the method comprising contacting an agent or a vector encoding the agent to the cell, whereby the agent modulates splicing of the first exon that comprises a translational start site from the pre-mRNA, thereby modulating the level of a processed mRNA that is processed from the pre-mRNA, and modulating the expression of the target protein in the cell, wherein the target gene is a SETD5 gene.

Described herein, in certain embodiments, is a method of treating or reducing the likelihood of developing a disease or condition in a subject in need thereof by modulating expression of a target protein in a cell of the subject, comprising: contacting an agent or a vector encoding the agent to the cell, whereby the agent modulates splicing of a first exon that comprises a translational start site from a pre-mRNA that is transcribed from a target gene and that comprises the first exon that comprises a translational start site, thereby modulating the level of a processed mRNA that is processed from the pre-mRNA, and modulating the expression of the target protein in the cell, wherein the target gene is a SETD5 gene.

In some embodiments, the first exon that comprises a translational start site is upstream of an NMD exon. In some embodiments, the NMD exon is upstream of the second exon that comprises a second translational start site.

In some embodiments, the target protein is SETD5.

In some embodiments, the agent: (a) binds to a targeted portion of the pre-mRNA; (b) modulates binding of a factor involved in splicing of the NMD exon; or (c) a combination of (a) and (b).

In some embodiments, the agent interferes with binding of the factor involved in splicing of the NMD exon or the first exon that comprises a translational start site to a region of the targeted portion.

In some embodiments, the targeted portion of the pre-mRNA is proximal to the NMD exon or the first exon that comprises a translational start site.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NMD exon or the first exon that comprises a translational start site.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NMD exon or the first exon that comprises a translational start site.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NMD exon or the first exon that comprises a translational start site.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NMD exon or the first exon that comprises a translational start site.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site selected from the group consisting of: GRCh38/hg38: chr3 9429827; GRCh38/hg38: chr3 9433370; GRCh38/hg38: chr3 9434591 and GRCh38/hg38: chr3 9428823 9429009.

In some embodiments, the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site selected from the group consisting of: GRCh38/hg38: chr3 9429827; GRCh38/hg38: chr3 9433370; GRCh38/hg38: chr3 9434591 and GRCh38/hg38: chr3 9428823 9429009.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr3 9430051; GRCh38/hg38: chr3 9433562; GRCh38/hg38: chr3 9434630 and GRCh38/hg38: chr3 9428823 9429009.

In some embodiments, the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr3 9430051; GRCh38/hg38: chr3 9433562; GRCh38/hg38: chr3 9434630 and GRCh38/hg38: chr3 9428823 9429009.

In some embodiments, the targeted portion of the pre-mRNA is located in an intronic region between two canonical exonic regions of the pre-mRNA, and wherein the intronic region contains the NMD exon.

In some embodiments, the targeted portion of the pre-mRNA at least partially overlaps with the NMD exon or the first exon that comprises a translational start site.

In some embodiments, the targeted portion of the pre-mRNA at least partially overlaps with an intron upstream or downstream of the NMD exon or the first exon that comprises a translational start site.

In some embodiments, the targeted portion of the pre-mRNA comprises a 5′ NMD exon exon-intron junction, a 3′NMD exon exon-intron junction, a 5′ first exon-intron junction, or a 3′ first exon-intron junction.

In some embodiments, the targeted portion of the pre-mRNA is within the NMD exon or the first exon that comprises a translational start site.

In some embodiments, the targeted portion of the pre-mRNA comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon or the first exon that comprises a translational start site.

In some embodiments, the NMD exon or the first exon that comprises a translational start site comprises a sequence with at least 80%, at least 90%, or 100% sequence identity to the sequence set forth in any one of SEQ ID NO: 332-339.

In some embodiments, the NMD exon or the first exon that comprises a translational start site comprises the sequence set forth in any one of SEQ ID NO: 332-339.

In some embodiments, the pre-mRNA comprises a sequence with at least about 80%, 85%, 90%, 95%, 97%, or 100% sequence identity to the sequence set forth in any one of SEQ ID NO: 332-403.

In some embodiments, the pre-mRNA is encoded by a genetic sequence with at least about 80%, 85%, 90%, 95%, 97%, or 100% sequence identity to the sequence set forth in any one of SEQ ID NO: 332-403.

In some embodiments, the targeted portion of the pre-mRNA comprises a sequence with at least 80%, 85%, 90%, 95%, 97%, or 100% sequence identity to a region comprising at least 8 contiguous nucleic acids of the sequence set forth in any one of SEQ ID NO: 332-403.

In some embodiments, the agent is an antisense oligomer (ASO) and wherein the ASO comprises a sequence that is at least about 80%, 85%, 90%, 95%, 97%, or 100% sequence identity to at least 8, 9, 10, 11, 12, 13, 14, 15, 16, 17 or 18 contiguous nucleic acids of the sequence set forth in any one of SEQ ID NO: 1-331.

In some embodiments, the method comprises contacting the vector encoding the agent to the cell, wherein the agent is a polynucleotide comprising an antisense oligomer.

In some embodiments, the vector is a viral vector.

In some embodiments, the viral vector is an adeno-associated viral vector.

In some embodiments, the polynucleotide further comprises a modified snRNA.

In some embodiments, the modified human snRNA is a modified U1 snRNA or a modified U7 snRNA.

In some embodiments, the modified human snRNA is a modified U7 snRNA and wherein the antisense oligomer has a sequence at least 75%, at least 80%, at least 85%, at least 90%, at least 95%, at least 98%, at least 99%, or 100% identical to the sequence set forth in any one of SEQ ID NO: 1-331.

In some embodiments, the targeted portion of the pre-mRNA is within the NMD exon or the first exon that comprises a translational start site selected from the group consisting of: GRCh38/hg38: chr3 9429827 9430051; GRCh38/hg38: chr3 9433370 9433562; GRCh38/hg38: chr3 9434591 9434630 and GRCh38/hg38: chr3 9428823 9429009.

In some embodiments, the targeted portion of the pre-mRNA is upstream or downstream of the NMD exon or the first exon that comprises a translational start site selected from the group consisting of: GRCh38/hg38: chr3 9429827 9430051; GRCh38/hg38: chr3 9433370 9433562; GRCh38/hg38: chr3 9434591 9434630 and GRCh38/hg38: chr3 9428823 9429009.

In some embodiments, the targeted portion of the pre-mRNA comprises an exon-intron junction of the NMD exon or the first exon that comprises a translational start site selected from the group consisting of: GRCh38/hg38: chr3 9429827 9430051; GRCh38/hg38: chr3 9433370 9433562; GRCh38/hg38: chr3 9434591 9434630 and GRCh38/hg38: chr3 9428823 9429009.

In some embodiments, the target protein expressed from the processed mRNA is a full-length protein or a wild-type protein.

In some embodiments, the target protein expressed from the processed mRNA is at least partially functional as compared to a wild-type SETD5 protein.

In some embodiments, the target protein expressed from the processed mRNA is at least partially functional as compared to a full-length wild-type SETD5 protein.

In some embodiments, the agent promotes exclusion of the NMD exon or the first exon that comprises a translational start site from the processed mRNA thereby modulating the level of a processed mRNA that is processed from the pre-mRNA and that lacks the NMD exon or the first exon that comprises a translational start site.

In some embodiments, the exclusion of the NMD exon or the first exon that comprises a translational start site from the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon or the first exon that comprises a translational start site from the processed mRNA in a control cell.

In some embodiments, the method results in an increase in the level of the processed mRNA in the cell.

In some embodiments, the level of the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA in a control cell.

In some embodiments, the agent increases the expression of the target protein in the cell.

In some embodiments, a level of the target protein expressed from the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein produced in a control cell.

In some embodiments, the disease or condition is associated with a loss-of-function mutation in the target gene or the target protein.

In some embodiments, the disease or condition is associated with haploinsufficiency of the target gene, and wherein the subject has a first allele of the target gene encoding a functional protein, and a second allele of the target gene from which the protein is not produced or produced at a reduced level, or a second allele of the target gene encoding a nonfunctional protein or a partially functional protein.

In some embodiments, the disease or condition is an intellectual disability or an autism spectrum disease.

In some embodiments, the disease or condition is selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21).

In some embodiments, the disease or condition is associated with an autosomal recessive mutation of a SETD5 gene, wherein the subject has a first allele encoding from which: (i) the target protein is not produced or produced at a reduced level compared to a wild-type allele; or (ii) the target protein produced is nonfunctional or partially functional compared to a wild-type allele, and a second allele from which: (iii) the target protein is produced at a reduced level compared to a wild-type allele and the target protein produced is at least partially functional compared to a wild-type allele; or (iv) the target protein produced is partially functional compared to a wild-type allele.

In some embodiments, the disease or condition is an intellectual disability or an autism spectrum disease.

In some embodiments, the disease or condition is selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21).

In some embodiments, the agent promotes exclusion of the NMD exon or the first exon that comprises a translational start site from the pre-mRNA, thereby modulating the level of a processed mRNA that is processed from the pre-mRNA and that lacks the NMD exon or the first exon that comprises a translational start site and increases the expression of the target protein in the cell.

In some embodiments, the agent is an antisense oligomer (ASO) and wherein the antisense oligomer comprises a backbone modification comprising a phosphorothioate linkage or a phosphorodiamidate linkage.

In some embodiments, the agent is an antisense oligomer (ASO) and wherein the antisense oligomer comprises a phosphorodiamidate morpholino, a locked nucleic acid, a peptide nucleic acid, a 2′-O-methyl, a 2′-Fluoro, a 2′-O-methoxyethyl moiety, or a 2′-NMA moiety.

In some embodiments, the agent is an antisense oligomer (ASO) and wherein the antisense oligomer comprises at least one modified sugar moiety.

In some embodiments, each sugar moiety is a modified sugar moiety.

In some embodiments, the agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some embodiments, the agent is an antisense oligomer (ASO) and wherein the antisense oligomer is at least 80%, at least 85%, at least 90%, at least 95%, at least 98%, at least 99%, or 100%, complementary to the targeted portion of the pre-mRNA.

In some embodiments, the method further comprises assessing processed mRNA level or expression level of the target protein.

In some embodiments, the subject is a human.

In some embodiments, the subject is a non-human animal.

In some embodiments, the subject is a fetus, an embryo, or a child.

In some embodiments, the cells are ex vivo.

In some embodiments, the agent is administered by intrathecal injection, intracerebroventricular injection, intraperitoneal injection, intramuscular injection, subcutaneous injection, intravitreal, or intravenous injection of the subject.

In some embodiments, the method further comprises administering a second therapeutic agent to the subject.

In some embodiments, the second therapeutic agent is a small molecule.

In some embodiments, the second therapeutic agent is an antisense oligomer.

In some embodiments, the second therapeutic agent corrects intron retention.

In some embodiments, the pre-mRNA comprises two or more NMD exons.

In some embodiments, the pre-mRNA comprises three or more NMD exons.

In some embodiments, splicing of one or more NMD exons from the pre-mRNA are modulated.

In some embodiments, splicing of two or more NMD exons from the pre-mRNA are modulated.

In some embodiments, splicing of three or more NMD exons from the pre-mRNA are modulated.

In some embodiments, the two or more NMD exons are located in a single intron.

In some embodiments, the two or more NMD exons are located in different introns.

In some embodiments, the three or more NMD exons are located in a single intron.

In some embodiments, the three or more NMD exons are located in different introns.

In some embodiments, the method treats the disease or condition.

In some embodiments, the NMD exon is an exon that encodes an amino acid sequence that comprises a cleavage site.

In some embodiments, the NMD exon is an exon that comprises a premature termination codon (PTC).

In some embodiments, the exon that comprises the PTC is an NMD exon.

In some embodiments, the NMD exon is downstream of a translation start site.

In some embodiments, the NMD exon does not comprise a translation start site.

In some embodiments, the NMD exon is an exon in a 5′UTR.

In some embodiments, the NMD exon is an exon in a 5′UTR that comprises a PTC.

Described herein, in certain embodiments, is a composition comprising an agent or a vector encoding the agent that modulates splicing of a non-sense mediated RNA decay-inducing exon (NMD exon) from a pre-mRNA that is transcribed from a target gene and that comprises the NMD exon, thereby modulating the level of a processed mRNA that is processed from the pre-mRNA, and modulating expression of a target protein in a cell having the pre-mRNA, wherein the target gene is a SETD5 gene. Described herein, in certain embodiments, is a composition comprising an agent or a vector encoding the agent that modulates splicing of a non-sense mediated RNA decay-inducing exon (NMD exon) from a pre-mRNA that is transcribed from a target gene and that comprises the NMD exon, thereby treating a disease or condition in a subject in need thereof by modulating the level of a processed mRNA that is processed from the pre-mRNA, and modulating expression of a target protein in a cell of the subject, wherein the target gene is a SETD5 gene.

Described herein, in certain embodiments, is a composition comprising an agent or a vector encoding the agent that modulates splicing of a first exon that comprises a translational start site from a pre-mRNA that is transcribed from a target gene and that comprises the first exon that comprises a translational start site, thereby modulating the level of a processed mRNA that is processed from the pre-mRNA, and modulating expression of a target protein in a cell having the pre-mRNA, wherein the target gene is a SETD5 gene.

Described herein, in certain embodiments, is a composition comprising an agent or a vector encoding the agent that modulates splicing of a first exon that comprises a translational start a pre-mRNA that is transcribed from a target gene and that comprises the first exon that comprises a translational start site, thereby treating a disease or condition in a subject in need thereof by modulating the level of a processed mRNA that is processed from the pre-mRNA, and modulating expression of a target protein in a cell of the subject, wherein the target gene is a SETD5 gene.

Described herein, in certain embodiments, is a pharmaceutical composition comprising the composition as described herein; and a pharmaceutically acceptable excipient and/or a delivery vehicle.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1B depict a schematic representation of a target mRNA that contains a non-sense mediated mRNA decay-inducing exon (NMD exon mRNA) and therapeutic agent-mediated exclusion of the nonsense-mediated mRNA decay-inducing exon to increase expression of the full-length target protein or functional RNA. FIG. 1A shows a cell divided into nuclear and cytoplasmic compartments. In the nucleus, a pre-mRNA transcript of a target gene undergoes splicing to generate mRNA, and this mRNA is exported to the cytoplasm and translated into target protein. For this target gene, some fraction of the mRNA contains a nonsense-mediated mRNA decay-inducing exon (NMD exon mRNA) that is degraded in the cytoplasm, thus leading to no target protein production. FIG. 1B shows an example of the same cell divided into nuclear and cytoplasmic compartments. Treatment with a therapeutic agent, such as an antisense oligomer (ASO), promotes the exclusion of the nonsense-mediated mRNA decay-inducing exon and results in an increase in mRNA, which is in turn translated into higher levels of target protein.

FIG. 2 depicts an SETD5 NMD-inducing exon inclusion event. NMD-inducing Exon 5X: UCSC Genome Browser snapshot of a region in the SETD5 gene (exons are rectangles and introns are lines with arrowheads) that contains an NMD-inducing exon inclusion event (chr3 9434591 9434630) depicted by the shaded area and black bar on the top. The transcript shown is NM_001080517. RNA sequencing traces from human middle frontal gyrus samples from individuals at various ages and proliferating or differentiated RenCell VM cells treated with cycloheximide (CHX) or DMSO control are shown.

FIG. 3A-FIG. 3D depict validation of an exon inclusion event (Exon 5X). FIG. 3A is a schematic representation of an Exon 5× inclusion event. Exon 5 and Exon 6 refer to transcript NM_001080517. FIG. 3B shows RT-PCR results using RNA from SK-N-AS (neuroblastoma), ReNcell VM cells (Neural progenitor cells), or U87-MG (likely gliobastoma) treated with either DMSO (−) or cycloheximide (CHX) (+). Primers were positioned in Exons 5 and 6 of the transcript NM_001080517. FIG. 3C shows quantification of the RT-PCR products using RNA from various 2-month-old mouse brain regions plotted as a percentage of exon inclusion isoform (Exon inc/(Exon inc+productive mRNA)*100). FIG. 3D shows quantification of the RT-PCR products using RNA from various non-human primate brain regions plotted as percentage of exon inclusion isoform (Exon inc/(Exon inc +productive mRNA)*100).

FIG. 4 depicts ASO walk design. The shaded nucleotides correspond to Exon 5X. Figure discloses SEQ ID NOS 511-513, respectively, in order of appearance.

FIG. 5A-FIG. 5C depict ASO walk TaqMan qPCR results, i.e., ASO screening by TaqMan qPCR. FIG. 5A shows TaqMan qPCR using RNA from ReNcell VM cells 24 hours after they were nucleofected with 2 μM ASOs from region 1 depicted in FIG. 4. The TaqMan probe spans the Exon 5 and Exon 6 junction (based on the transcript NM_001080517) and measures productive mRNA. FIG. 5B shows TaqMan qPCR using RNA from ReNcell VM cells 24 hours after they were nucleofected with 2 μM ASOs from region 2 depicted in FIG. 4. The TaqMan probe spans the Exon 5 and Exon 6 junction (based on the transcript NM_001080517) and measures productive mRNA. FIG. 5C shows TaqMan qPCR using RNA from ReNcell VM cells nucleofected for 24 hours with 2 M ASOs from region 3 depicted in FIG. 4. The TaqMan probe spans the Exon 5 and Exon 6 junction (based on the transcript NM_001080517) and measures productive mRNA.

FIG. 6 depicts SETD5 NMD-inducing Exons 3× and 3Y inclusion events. UCSC Genome Browser snapshot of a region in the SETD5 gene (exons are rectangles and introns are lines with arrowheads) that contains NMD-inducing exon 3× (chr3 9429827 9430051) and NMD-inducing exon 3Y (chr3 9433370 9433562) depicted by the shaded area and black bar on the top. The transcript shown is NM_001080517. RNA sequencing traces from human middle frontal gyrus samples from individuals at various ages and proliferating ReNcell VM cells treated with cycloheximide (CHX) or DMSO control are shown.

FIG. 7A-FIG. 7D depict validation of Exons 3X and 3Y inclusion events. FIG. 7A shows schematic representation of Exons 3X and 3Y inclusion events. Exon 3 and Exon 4 refer to transcript NM_001080517. FIG. 7B shows RT-PCR results using RNA from SK-N-AS (neuroblastoma), ReNcell VM cells (Neural progenitor cells), or U87-MG (likely glioblastoma) treated with either DMSO (−) or cycloheximide (CHX) (+). Primers were positioned in Exons 3 and 4 of the transcript NM_001080517. FIG. 7C shows quantification of the RT-PCR products using RNA from various 2-month-old mouse brain regions plotted as percentage of exon inclusion isoform (Exon inc/(Exon inc+productive mRNA)*100). FIG. 7D shows quantification of the RT-PCR products using RNA from various non-human primate brain regions plotted as a percentage of exon inclusion isoform (Exon inc/(Exon inc +productive mRNA)*100).

FIG. 8 depicts ASO walk design for Exon 3X. The shaded nucleotides correspond to the Exon 3X. Figure discloses SEQ ID NOS 514-516, respectively, in order of appearance.

FIG. 9 depicts ASO walk design for Exon 3Y. The shaded nucleotides correspond to the Exon 3Y. Figure discloses SEQ ID NOS 517-519, respectively, in order of appearance.

FIG. 10 depicts ASO walk design for Exon 3. The shaded nucleotides correspond to the Exon 3. Figure discloses SEQ ID NOS 520-522, respectively, in order of appearance.

FIG. 11 shows the turning of SETD5 NMD exons into 5′UTR exons. The black boxes denote NMD exons. Exons that are half the height refer to exons that are not translated. Exons that are full height refer to exons that are translated.

FIG. 12 shows relative fold changes in the levels of SETD5 RNAs in response to the treatment of different exemplary ASOs according to some embodiments of the present disclosure or non-targeting ASO control (“NTC”), as compared to mock control, as assessed by TaqMan qPCR reactions that used primers spanning canonical Exon 5 and Exon 6 junction (“canonical (5-6)”), NMD Exon 5X (“NMD Exon (5X)”), and Exon 13 and Exon 14 junction (“Downstream (13-14)”).

FIGS. 13A and 13B show SETD5 protein expression level changes in response to ASO treatment, as measured by Jess Western blotting. Briefly, ReNcell VM cells were transfected with different exemplary ASOs according to some embodiments of the present disclosure, non-targeting ASO control (“NTC”), or no ASO (“mock control”), in the absence of cycloheximide. 72 hours after transfection, the cells were lysed, and the protein lysates were analyzed for impact on SETD5 protein expression by Jess Western blotting. FIG. 13A shows a representative Jess Western blot image of SETD5 protein in different experimental conditions, together with an image of total protein level as a loading control. FIG. 13B is a plot summarizing the fold change in SETD5 protein level as assessed by Jess Western blot and normalized to the total protein level. As shown in the figure, all tested ASOs were shown to increase the protein level of SETD5 in the cells.

FIG. 14 depicts the ASO macrowalk design for SETD5 Exon 5X.

FIGS. 15A-15B represent summative data at the 24-hour timepoint after nucleofection of HEK293 cells with 1 μM SETD5 macrowalk ASOs of FIG. 14. FIG. 15A shows a histogram representing the fold difference in target engagement of various ASOs from the SETD5 macrowalk relative to a mock control. FIG. 15B is a graph depicting the fold change of gene expression by various ASOs from the SETD5 macrowalk relative to a mock control.

FIGS. 16A-16B represent summative data at the 3-day timepoint after free-uptake of 20 M SETD5 macrowalk ASOs of FIG. 14 by HEK293 cells. FIG. 16A shows a histogram representing the fold difference in target engagement of various ASOs from the SETD5 macrowalk relative to control samples. FIG. 15B is a graph depicting the fold change of gene expression by various ASOs from the SETD5 macrowalk relative to control samples.

FIGS. 17A-17B illustrate summative data at the 24-hour timepoint after nucleofection of HEK293 cells with 0.5 M SETD5 macrowalk ASOs. FIG. 17A shows a histogram representing the percentage of SETD5 Exon 5X inclusion in HEK293 cells post application of various ASOs. FIG. 17B is a graph depicting the fold change of gene expression by various ASOs from the SETD5 macrowalk relative to control samples.

FIGS. 18A-18B represent summative data at the 3-day timepoint after free-uptake of 10 M SETD5 macrowalk ASOs by HEK293 cells. FIG. 18A shows a histogram representing the percentage of SETD5 Exon 5X inclusion in HEK293 cells post application of various ASOs. FIG. 18B is a graph depicting the fold change of gene expression by various ASOs from the SETD5 macrowalk relative to control samples.

FIGS. 19A-19B represent summative data at the 24-hour timepoint after nucleofection of HEK293 cells with 1 μM SETD5 microwalk ASOs. FIG. 19A shows a histogram representing the percentage of SETD5 Exon 5X inclusion in HEK293 cells post application of various ASOs relative to mock and SMN (SMN1 survival of motor neuron 1) targeting ASO controls. FIG. 19B is a graph representing the fold change of gene expression by various ASOs from a SETD5 microwalk relative to control samples.

FIGS. 20A-20B represent summative data at the 3-day timepoint after free-uptake of 20 μM SETD5 microwalk ASOs by HEK293 cells. FIG. 20A shows a histogram representing the percentage of SETD5 Exon 5X inclusion in HEK293 cells post application of various ASOs relative to mock and SMN controls. FIG. 20B is a graph representing the fold change of gene expression by various ASOs from a SETD5 microwalk relative to control samples.

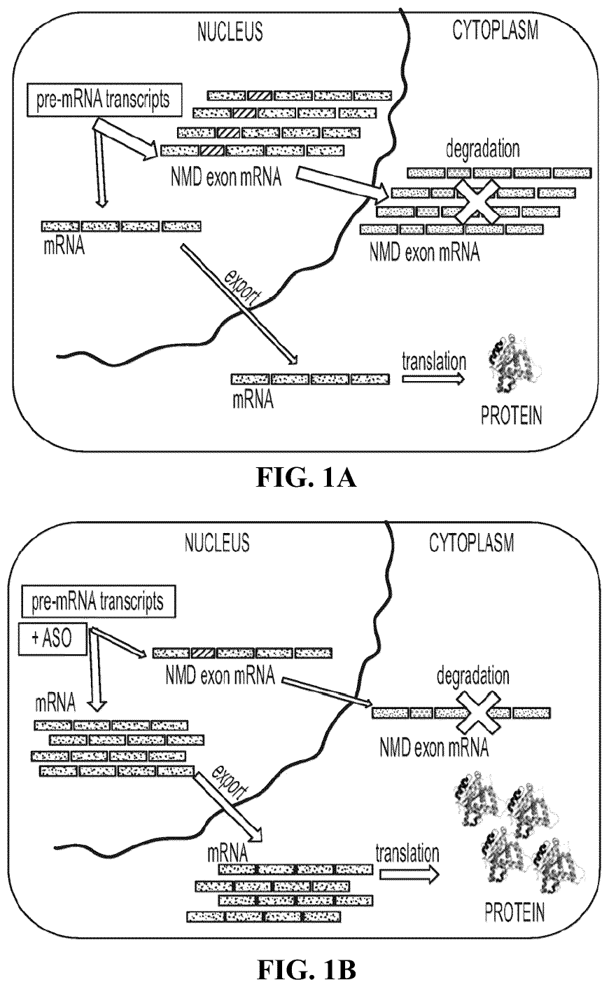

### DETAILED DESCRIPTION

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The coordinate as used herein refers to the coordinate of the genome reference assembly GRCh38 (Genome Research Consortium human build 38), also known as Hg38 (Human genome build 38).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

Alternative splicing events in SETD5 gene can lead to non-productive mRNA transcripts which in turn can lead to aberrant protein expression, and therapeutic agents which can target the alternative splicing events in SETD5 gene can modulate the expression level of functional proteins in DS patients and/or inhibit aberrant protein expression. Such therapeutic agents can be used to treat a condition caused by SETD5 protein deficiency.

One of the alternative splicing events that can lead to non-productive mRNA transcripts is the inclusion of an extra exon in the mRNA transcript that can induce non-sense mediated mRNA decay. The present disclosure provides compositions and methods for modulating alternative splicing of SETD5 to increase the production of protein-coding mature mRNA, and thus, translated functional SETD5 protein. These compositions and methods include antisense oligomers (ASOs) that can cause exon skipping and promote constitutive splicing of SETD5 pre-mRNA. In various embodiments, functional SETD5 protein can be increased using the methods of the disclosure to treat a condition caused by SETD5 protein deficiency.

“SETD5,” also known as SET domain containing 5, Histone-lysine N-methyltransferase SETD5, as referred to herein, includes any of the recombinant or naturally occurring forms of SETD5 protein or variants or homologs thereof that maintain SETD5 activity (e.g., within at least 40% 50%, 60%, 70%, 80%, 90%, 95%, 96%, 97%, 98%, 99% or 100% activity compared to SETD5). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring SETD5 protein. In some embodiments, the SETD5 protein is substantially identical to the protein identified by the UniProt reference number Q9C0A6 or a variant or homolog having substantial identity thereto.

Splicing and Nonsense-Mediated mRNA Decay

Intervening sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of the 5′ splice site (5′ss) by U1 snRNA or the 3′splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency.

Those mRNA isoforms with premature termination codons (PTCs) at least 50 bp upstream of an exon junction complex are likely to be targeted for degradation by the nonsense-mediated mRNA decay (NMD) pathway. Mutations in traditional (BPS/PPT/3′ss/5′ss) and auxiliary splicing motifs can cause aberrant splicing, such as exon skipping or cryptic (or pseudo-) exon inclusion or splice-site activation and contribute significantly to human morbidity and mortality. Both aberrant and alternative splicing patterns can be influenced by natural DNA variants in exons and introns.

Given that exon-intron boundaries can occur at any of the three positions of a codon, it is clear that only a subset of alternative splicing events can maintain the canonical open reading frame. For example, only exons that are evenly divisible by 3 can be skipped or included in the mRNA without any alteration of reading frame. Splicing events that do not have compatible phases will induce a frame-shift. Unless reversed by downstream events, frame-shifts can certainly lead to one or more PTCs, probably resulting in subsequent degradation by NMD. NMD is a translation-coupled mechanism that eliminates mRNAs containing PTCs. NMD can function as a surveillance pathway that exists in all eukaryotes. NMD can reduce errors in gene expression by eliminating mRNA transcripts that contain premature stop codons. Translation of these aberrant mRNAs could, in some cases, lead to deleterious gain-of-function or dominant-negative activity of the resulting proteins. NMD targets not only transcripts with PTCs but also a broad array of mRNA isoforms expressed from many endogenous genes, suggesting that NMD is a master regulator that drives both fine and coarse adjustments in steady-state RNA levels in the cell.

The terms “non-sense mediated RNA decay exon” (or “NSE” or “NMD exon”) and “NMD-inducing exon” (or NIE) used interchangeably and can refer to an exon (e.g., a noncanonical exon) that can activate the NMD pathway if present in a mature RNA transcript. A fully processed mRNA that contains an NMD exon can be subject to non-sense mediated degradation (NMD). In constitutive splicing events, the intron containing an NMD exon is usually spliced out, but the intron or a portion thereof (e.g., NMD exon) may be retained during alternative or aberrant splicing events. Mature mRNA transcripts containing an NMD exon may be non-productive, for example, due to frame shifts which induce the NMD pathway. A mature mRNA transcript that contains an NMD exon can contain a premature stop codon (or premature termination codon (PTC)). In some embodiments, a mature mRNA transcript containing an NMD exon contains a premature stop codon (or premature termination codon (PTC)) downstream of the NMD exon. In some embodiments, an NMD exon is an exon that contains a premature stop codon (or premature termination codon (PTC)) or other sequences that facilitate degradation of a mature RNA transcript containing the NMD exon. Inclusion of a NMD exon in mature RNA transcripts may downregulate gene expression. An NMD exon can be within an intron of a pre-mRNA. In some embodiments, an NMD exon is a region within an intron (e.g., a canonical intron). In some embodiments, an NMD exon is downstream from a translation start site. For example, an NMD exon can be an exon downstream of an exon containing a translation start site (e.g., a first translation start site). In some embodiments, an NMD exon is not an exon that contains a translation start site. In some embodiments, an NMD exon is an exon in a 5′ UTR.

The terms “mature mRNA,” and “fully-spliced mRNA,” are used interchangeably herein to describe a fully processed mRNA. A mature mRNA that contains an NMD exon can be non-productive mRNA and lead to NMD of the mature mRNA. NMD exon containing mature mRNA may sometimes lead to reduced protein expression compared to protein expression from a corresponding mature mRNA that does not contain the NMD exon.

In some embodiments, the compositions as described herein or the methods as described herein increase protein expression by increasing productive RNA production. In some embodiments, by the compositions as described herein or the methods as described herein, translation starts at the second translation start site that is downstream of NMD causing events by skipping the exon containing the first translation start site that is upstream of NMD causing events. In some embodiments, the compositions as described herein or the methods as described herein shift the translation start site from the first translation initiation site to the second translation initiation site. In some embodiments, the compositions as described herein or the methods as described herein transform the exon containing the first translation start site that is upstream of NMD causing events to the UTR (untranslated region). In some embodiments, the compositions as described herein or the methods as described herein transform the exon containing the first translation start site that is upstream of NMD causing events to the 5′-UTR. In some embodiments, the compositions as described herein or the methods as described herein lead to skipping an upstream ORF (open reading frame) to start translation downstream of NMD causing events to avoid inclusion of PTC-containing exons. In some embodiments, the compositions as described herein or the methods as described herein lead to shifting translation start sites to avoid NMD exons. In some embodiments, the compositions as described herein or the methods as described herein lead to skipping an exon upstream of a NMD exon, wherein there is a translation start site downstream of the NMD exon.

In some embodiments, the protein translated from the second translation start site has the activity same as or comparable to the protein translated from the first translation start site. In some embodiments, the protein translated from the second translation start site has the activity within at least 40% 50%, 60%, 70%, 80%, 90%, 95%, 96%, 97%, 98%, 99% or 100% activity compared to the protein translated from the first translation start site.

The “translation start site” or “translation initiation site” as used herein refers to the location where translation into protein starts. The start codon is the first codon of a messenger RNA (mRNA) transcript translated by a ribosome. The start codon codes for methionine in eukaryotes and Archaea, and a N-formylmethionine (fMet) in bacteria, mitochondria, and plastids. In some embodiments, the start codon is preceded by a 5′ untranslated region (5′ UTR).

Pseudo splice sites have the same splicing recognition sequences as genuine splice sites but are not used in splicing reactions. They outnumber genuine splice sites in the human genome by an order of a magnitude and are normally repressed by thus far poorly understood molecular mechanisms. Cryptic 5′ splice sites have the consensus NNN/GUNNNN or NNN/GCNNNN where N is any nucleotide and/is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where M is C or A, R is G or A, and Y is C or U.

Splice sites and their regulatory sequences can be readily identified by a skilled person using suitable algorithms publicly available, listed, for example, in Kralovicova, J. and Vorechovsky, I. (2007) Global control of aberrant splice site activation by auxiliary splicing sequences: evidence for a gradient in exon and intron definition. Nucleic Acids Res., 35, 6399-6413, (nebi.nlm.nih.gov/pmc/articles/PMC2095810/pdf/gkm680.pdf)

### Target Genes

The present disclosure provides compositions and methods for modulating alternative splicing of a target to modulate the production of functional protein-coding mature mRNA, and thus, translated functional the target protein, wherein the target is SETD5. These compositions and methods include antisense oligomers (ASOs) that can promote canonical splicing of the target pre-mRNA, wherein the target is SETD5. In various embodiments, functional target protein can be increased using the methods of the disclosure to treat a condition caused by target protein deficiency, wherein the target is any one selected from the group consisting of SETD5.

In some embodiments, the methods of the invention are used to increase functional the target protein production to treat a condition in a subject in need thereof, wherein the target gene is SETD5 gene. In some embodiments, the subject has a condition in which the target protein is not necessarily deficient relative to wild-type, but where an increase in the target protein mitigates the condition nonetheless, wherein the target gene is SETD5 gene. In some embodiments, the condition is caused by sporadic mutation. In some embodiments, the methods of the invention are used to reduce functional target protein production to treat a condition in a subject in need thereof, wherein the target gene is SETD5 gene. In some embodiments, the methods of the invention are used to modulate functional target protein production to treat a condition in a subject in need thereof, wherein the target is gene is SETD5 gene.

### Target Transcripts

In some embodiments, the methods of the present disclosure exploit the presence of NIE in the pre-mRNA transcribed from SETD5 genes. Splicing of the identified SETD5 NIE pre-mRNA species to produce functional mature SETD5 mRNA may be induced using a therapeutic agent such as an ASO that stimulates exon skipping of an NIE. Induction of exon skipping may result in inhibition of an NMD pathway. The resulting mature SETD5 mRNA can be translated normally without activating NMD pathway, thereby increasing the amount of SETD5 protein in the patient's cells and alleviating symptoms of a condition or disease associated with SETD5 deficiency, such as an intellectual disability or an autism spectrum disease. the disease or condition is selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21).

Canonical splicing of the identified target NSAE (non-sense mediated RNA decay alternative exon) from pre-mRNA transcripts to produce the functional, mature target mRNA can be induced using a therapeutic agent, such as an ASO, that promotes constitutive splicing of the target NSAE pre-mRNA at the canonical splice sites. In some embodiments, the resulting functional, mature target mRNA can be translated normally, thereby increasing the amount of the functional target protein in the patient's cells and preventing symptoms of the target associated disease. In some embodiments, canonical splicing of the identified target NSAE pre-mRNA transcripts to produce functional, mature target mRNA may be reduced using a therapeutic agent, such as an ASO, that inhibits constitutive splicing of target NSAE pre-mRNA at the canonical splice sites. In some embodiments, the resulting functional, mature target mRNA can be translated abnormally, thereby decreasing the amount of functional target protein in the patient's cells and preventing symptoms of the target associated disease.

In some embodiments, the diseases or conditions that can be treated or ameliorated using the method or composition disclosed herein are not directly associated with the target protein (gene) that the therapeutic agent targets. In some embodiments, a therapeutic agent provided herein can target a protein (gene) that is not directly associated with a disease or condition, but the modulation of expression of the target protein (gene) can treat or ameliorate the disease or condition. For instance, targeting genes like SETD5 by a therapeutic agent provided herein can treat or ameliorate can treat or ameliorate central nervous system diseases. For instance, targeting genes like SETD5 by a therapeutic agent provided herein can treat or ameliorate intellectual disability or an autism spectrum disease. In some embodiments, targeting genes like SETD5 by a therapeutic agent provided herein can treat or ameliorate a disease or condition selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21). In some embodiments, such target genes like SETD5 are said to be indicated for Pathway (central nervous system). In some embodiments, such target genes like SETD5 are said to be indicated for Pathway (intellectual disability or an autism spectrum disease). In some embodiments, such target genes like SETD5 are said to be indicated for Pathway (autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21)).

In various embodiments, the present disclosure provides a therapeutic agent which can target SETD5 mRNA transcripts to modulate splicing or protein expression level. The therapeutic agent can be a small molecule, polynucleotide, or polypeptide. In some embodiments, the therapeutic agent is an ASO. Various regions or sequences on the SETD5 pre-mRNA can be targeted by a therapeutic agent, such as an ASO. In some embodiments, the ASO targets a SETD5 pre-mRNA transcript containing an NIE. In some embodiments, the ASO targets a sequence within an NIE of a SETD5 pre-mRNA transcript. In some embodiments, the ASO targets a sequence upstream (or 5′) from the 5′ end of an NIE (3′ss) of a SETD5 pre-mRNA transcript. In some embodiments, the ASO targets a sequence downstream (or 3′) from the 3′ end of an NIE (5′ss) of a SETD5 pre-mRNA transcript. In some embodiments, the ASO targets a sequence that is within an intron flanking on the 5′ end of the NIE of a SETD5 pre-mRNA transcript. In some embodiments, the ASO targets a sequence that is within an intron flanking the 3′ end of the NIE of a SETD5 pre-mRNA transcript. In some embodiments, the ASO targets a sequence comprising an NIE-intron boundary of a SETD5 pre-mRNA transcript. An NIE-intron boundary can refer to the junction of an intron sequence and an NIE region. The intron sequence can flank the 5′ end of the NIE, or the 3′ end of the NIE. In some embodiments, the ASO targets a sequence within an exon of a SETD5 pre-mRNA transcript. In some embodiments, the ASO targets a sequence within an intron of a SETD5 pre-mRNA transcript. In some embodiments, the ASO targets a sequence comprising both a portion of an intron and a portion of an exon of a SETD5 pre-mRNA transcript.

In some embodiments, the ASO targets a sequence about 4 to about 300 nucleotides upstream (or 5′) from the 5′ end of the NIE. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 5′ end of the NIE region. In some embodiments, the ASO may target a sequence more than 300 nucleotides upstream from the 5′ end of the NIE. In some embodiments, the ASO targets a sequence about 4 to about 300 nucleotides downstream (or 3′) from the 3′ end of the NIE. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 3′ end of the NIE. In some embodiments, the ASO targets a sequence more than 300 nucleotides downstream from the 3′ end of the NIE.

In some embodiments, the ASOs disclosed herein target a NSAE pre-mRNA transcribed from SETD5 genomic sequence. In some embodiments, the ASO targets a NSAE pre-mRNA transcript from a genomic sequence comprising a NSAE exon of SETD5 genomic sequences. In some embodiments, the ASO targets a NSAE pre-mRNA transcript from a genomic sequence comprising an intron flanking the 3′ splice site of the NSAE exon and an intron flanking the 5′ splice site of a NSAE exon of SETD5 genomic sequences. In some embodiments, the ASO targets a NSAE pre-mRNA transcript comprising a sequence selected from the group consisting of the pre-mRNA transcripts of Table 3 (e.g., the sequence set forth in any one of SEQ ID NO: 340-403). In some embodiments, the ASO targets a pre-mRNA sequence comprising a NSAE exon of SETD5 pre-mRNA sequences. In some embodiments, the ASO targets a pre-mRNA sequence comprising an intron flanking the 3′ splice site of the NSAE exon of SETD5 pre-mRNA sequences. In some embodiments, the ASO targets a pre-mRNA sequence comprising an intron flanking the 5′ splice site of the NSAE exon of SETD5 pre-mRNA sequences. In some embodiments, the transcript is selected from the group consisting of the transcripts of Table 3 (e.g., the sequence set forth in any one of SEQ ID NO: 340-403).

In some embodiments, the SETD5 NIE containing pre-mRNA transcript is encoded by a genetic sequence with at least about 80%, 85%, 90%, 95%, 96%, 97%, 98%, 99% or 100% sequence identity to any one of the sequences listed in Table 2 or Table 3 (e.g., the sequence set forth in any one of SEQ ID NO: 332-403). In some embodiments, the SETD5 NIE pre-mRNA transcript comprises a sequence with at least about 80%, 85%, 90%, 95%, 96%, 97%, 98%, 99% or 100% sequence identity to any one of the sequences listed in Table 2 or Table 3 (e.g., the sequence set forth in any one of SEQ ID NO: 332-403).

In some embodiments, the SETD5 NIE containing pre-mRNA transcript (or NMD exon mRNA) comprises a sequence with at least about 80%, 85%, 90%, 95%, 97%, or 100% sequence identity to any one of the sequences listed in Table 2 or Table 3 (e.g., the sequence set forth in any one of SEQ ID NO: 332-403). In some embodiments, SETD5 NIE containing pre-mRNA transcript (or NMD exon mRNA) is encoded by a sequence with at least about 80%, 85%, 90%, 95%, 97%, or 100% sequence identity to any one of the sequences listed in Table 2 or Table 3 (e.g., the sequence set forth in any one of SEQ ID NO: 332-403). In some embodiments, the targeted portion of the NMD exon mRNA comprises a sequence with at least 80%, 85%, 90%, 95%, 97%, or 100% sequence identity to a region comprising at least 8 contiguous nucleic acids of any one of the sequences listed in Table 2 or Table 3 (e.g., the sequence set forth in any one of SEQ ID NO: 332-403).

In some embodiments, the pre-mRNA transcript comprises a sequence with at least about 80%, 85%, 90%, 95%, 96%, 97%, 98%, 99% or 100% sequence identity to a pre-mRNA transcript of SETD5 pre-mRNA transcripts or a complement thereof described herein. In some embodiments, the targeted portion of the pre-mRNA selected from the group consisting of SETD5 pre-mRNAs comprises a sequence with at least 80%, 85%, 90%, 95%, 97%, or 100% sequence identity to a region comprising at least 8 contiguous nucleotides of a sequence of the pre-mRNA transcripts of Table 2 or Table 3 (e.g., the sequence set forth in any one of SEQ ID NO: 332-403) or complements thereof. In some embodiments, the targeted portion of the pre-mRNA of SETD5 pre-mRNA comprises a sequence that is complementary to at least 8, 9, 10, 11, 12, 13, 14, 15, 16, 17, 18, 19 or 20 contiguous nucleic acids of a sequence of Table 2 or Table 3 (e.g., the sequence set forth in any one of SEQ ID NO: 332-403) or a complement thereof.

In some embodiments, the pre-mRNA transcript comprises a sequence with at least about 80%, 85%, 90%, 95%, 96%, 97%, 98%, 99% or 100% sequence identity to a pre-mRNA transcript of SETD5 pre-mRNA transcripts or a complement thereof described herein. In some embodiments, the targeted portion of the pre-mRNA selected from the group consisting of SETD5 pre-mRNAs comprises a sequence with at least 80%, 85%, 90%, 95%, 97%, or 100% sequence identity to a region comprising at least 8 contiguous nucleotides of a sequence of the pre-mRNA transcripts of Table 2 or Table 3 (e.g., the sequence set forth in any one of SEQ ID NO: 332-403) or complements thereof. In some embodiments, the targeted portion of the pre-mRNA of SETD5 pre-mRNA comprises a sequence that is complementary to at least 8, 9, 10, 11, 12, 13, 14, 15, 16, 17, 18, 19 or 20 contiguous nucleic acids of a sequence of Table 2 or Table 3 (e.g., the sequence set forth in any one of SEQ ID NO: 332-403) or a complement thereof.

In some embodiments, the ASOs disclosed herein target a NSAE pre-mRNA transcribed from a SETD5 genomic sequence. In some embodiments, the ASO targets a NSAE pre-mRNA transcript from a SETD5 genomic sequence comprising a NSAE exon.

In some embodiments, the SETD5 pre-mRNA transcript is encoded by a genetic sequence with at least about 80%, 85%, 90%, 95%, 96%, 97%, 98%, 99% or 100% sequence identity to the Ensembl reference number ENSG00000168137.18 or a complement thereof. In some embodiments, the SETD5 pre-mRNA transcript comprises a sequence with at least about 80%, 85%, 90%, 95%, 96%, 97%, 98%, 99% or 100% sequence identity to a SETD5 pre-mRNA transcript or a complement thereof described herein.

In some embodiments, the targeted portion of the SETD5 pre-mRNA comprises a sequence with at least 80%, 85%, 90%, 95%, 97%, or 100% sequence identity to a region comprising at least 8 contiguous nucleotides of a sequence of sequence of Table 3 (e.g., the sequence set forth in any one of SEQ ID NO: 340-403) or complements thereof. In some embodiments, the targeted portion of the SETD5 pre-mRNA comprises a sequence with at least 80%, 85%, 90%, 95%, 97%, or 100% sequence identity to a region comprising at least 8 contiguous nucleotides of a sequence selected from the group consisting of sequences listed in Table 2 or Table 3 (e.g., the sequence set forth in any one of SEQ ID NO: 332-403) or complements thereof. In some embodiments, the ASO comprises a sequence that is at least about 80%, 85%, 90%, 95%, 97%, or 100% identical to any one the sequences of Table 4 (e.g., the sequence set forth in any one of SEQ ID NO: 1-331) or complements thereof.

In some embodiments, the ASO targets Exon 5X of a SETD5 pre-mRNA. In some embodiments, the ASO targets exon 3X and exon 3Y of a SETD5 pre-mRNA. In some embodiments, the ASO targets exon 3X or exon 3Y of a SETD5 pre-mRNA. In some embodiments, the ASO targets exon 3X of a SETD5 pre-mRNA. In some embodiments, the ASO targets exon 3Y of a SETD5 pre-mRNA.

In some embodiments, the ASO targets exon (GRCh38/hg38: chr3 9429827 9430051), exon (GRCh38/hg38: chr3 9433370 9433562), exon (GRCh38/hg38: chr3 9434591 9434630) or exon (GRCh38/hg38: chr3 9428823 9429009) of SETD5.

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of Exon 5X, exon 3X, or exon 3Y, of SETD5. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr3 9429827 9430051, GRCh38/hg38: chr3 9433370 9433562, GRCh38/hg38: chr3 9434591 9434630 or GRCh38/hg38: chr3 9428823 9429009 of SETD5.

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of Exon 5X, exon 3X, or exon 3Y of SETD5. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr3 9429827 9430051, GRCh38/hg38: chr3 9433370 9433562, GRCh38/hg38: chr3 9434591 9434630 or GRCh38/hg38: chr3 9428823 9429009 of SETD5.

In some embodiments, the ASO has a sequence complementary to the targeted portion of the NMD exon mRNA according to any one of the sequences listed in Table 2 or Table 3 (e.g., the sequence set forth in any one of SEQ ID NO: 332-403).

In some embodiments, the ASO targets a sequence upstream from the 5′ end of an NIE. For example, ASOs targeting a sequence upstream from the 5′ end of an NIE (e.g., Exon 5X, exon 3X, exon 3Y, of SETD5) comprises a sequence that is at least about 80%, 85%, 90%, 95%, 97%, or 100% complimentary to at least 8 contiguous nucleic acids of any one of the sequences listed in Table 2 or Table 3 (e.g., the sequence set forth in any one of SEQ ID NO: 332-403). For example, ASOs targeting a sequence upstream from the 5′ end of an NIE (e.g., exon (GRCh38/hg38: chr3 9429827 9430051), exon (GRCh38/hg38: chr3 9433370 9433562), exon (GRCh38/hg38: chr3 9434591 9434630) or exon (GRCh38/hg38: chr3 9428823 9429009) of SETD5) can comprise a sequence with at least 80%, 85%, 90%, 95%, 97%, or 100% sequence identity to any one of the sequences listed in Table 2 or Table 3 (e.g., the sequence set forth in any one of SEQ ID NO: 332-403).

In some embodiments, the ASOs target a sequence containing an exon-intron boundary (or junction). For example, ASOs targeting a sequence containing an exon-intron boundary can comprise a sequence that is at least about 80%, 85%, 90%, 95%, 97%, or 100% complimentary to at least 8 contiguous nucleic acids of any one of the sequences listed in Table 2 or Table 3 (e.g., the sequence set forth in any one of SEQ ID NO: 332-403). In some embodiments, the ASOs target a sequence downstream from the 3′ end of an NIE. For example, ASOs targeting a sequence downstream from the 3′ end of an NIE (e.g., Exon 5X, exon 3X, exon 3Y, of SETD5) can comprise a sequence with at least 80%, 85%, 90%, 95%, 97%, or 100% sequence identity to any one of the sequences listed in Table 2 or Table 3 (e.g., the sequence set forth in any one of SEQ ID NO: 332-403). For example, ASOs targeting a sequence downstream from the 3′ end of an NIE (e.g., exon (GRCh38/hg38: chr3 9429827 9430051), exon (GRCh38/hg38: chr3 9433370 9433562), exon (GRCh38/hg38: chr3 9434591 9434630) or exon (GRCh38/hg38: chr3 9428823 9429009) of SETD5) can comprise a sequence with at least 80%, 85%, 90%, 95%, 97%, or 100% sequence identity to any one of the sequences listed in Table 2 or Table 3 (e.g., the sequence set forth in any one of SEQ ID NO: 332-403). In some embodiments, ASOs target a sequence within an NIE.

In some embodiments, the ASO targets Exon 5X of a SETD5 NIE containing pre-mRNA comprising NIE Exon 5X, exon 3X of a SETD5 NIE containing pre-mRNA comprising NIE exon 3X, or exon 3Y of a SETD5 NIE containing pre-mRNA comprising NIE exon 3Y. In some embodiments, the ASO targets a sequence downstream (or 3′) from the 5′ end of Exon 5X, exon 3X, or exon 3Y of SETD5 pre-mRNA. In some embodiments, the ASO targets an exon sequence upstream (or 5′) from the 3′ end of Exon 5X, exon 3X or exon 3Y of SETD5 pre-mRNA.

In some embodiments, the targeted portion of the SETD5 NIE containing pre-mRNA is in intron 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, hybridization of an ASO to the targeted portion of the NIE pre-mRNA results in exon skipping of at least one of NIE within intron 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, and subsequently increases SETD5 protein production. In some embodiments, the targeted portion of the SETD5 NIE containing pre-mRNA is in canonical intron 5 or canonical intron 3 of SETD5. In some embodiments, the targeted portion of the SETD5 NIE containing pre-mRNA is intron (GRCh38/hg38:chr3 9434486 9434823) or intron (GRCh38/hg38: chr3 9429010 9433844) of SETD5.

In some embodiments, the methods and compositions of the present disclosure are used to increase the expression of SETD5 by inducing exon skipping of a NMD exon of an SETD5 NIE containing pre-mRNA. In some embodiments, the NMD exon is a sequence within any of introns 1-50. In some embodiments, the NMD exon is a sequence within any of introns 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, the NMD exon can be any SETD5 intron or a portion thereof. In some embodiments, the NMD exon is within canonical intron 5 or canonical intron 3 of SETD5. In some embodiments, the NMD exon is within intron (GRCh38/hg38:chr3 9434486 9434823) or intron (GRCh38/hg38: chr3 9429010 9433844) of SETD5. In some embodiments, the NMD exon is canonical exon 3.

### Protein Expression

In some embodiments, a mutation occurs in both alleles. In some embodiments, a mutation occurs in one of the two alleles. In some embodiments, additional mutation occurs in one of the two alleles. In some embodiments, the additional mutation occurs in the same allele as the first mutation. In other embodiments, the additional mutation occurs is a trans mutation.

In some embodiments, the methods described herein are used to increase the production of a functional SETD5 protein or SETD5 RNA. As used herein, the term “functional” refers to the amount of activity or function of a SETD5 protein or SETD5 RNA that is necessary to eliminate any one or more symptoms of a treated condition or disease, e.g., an intellectual disability or an autism spectrum disease. In some embodiments, the disease or condition is selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21). In some embodiments, the methods are used to increase the production of a partially functional SETD5 protein or SETD5 RNA. As used herein, the term “partially functional” refers to any amount of activity or function of the SETD5 protein or SETD5 RNA that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the SETD5 protein by cells of a subject having a NIE containing pre-mRNA encoding SETD5 protein, wherein the subject has an intellectual disability or an autism spectrum disease caused by a deficient amount of activity of SETD5 protein, and wherein the deficient amount of the SETD5 protein is caused by haploinsufficiency of the SETD5 protein. In some embodiments, the subject has a disease or condition selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21). In such an embodiment, the subject has a first allele encoding a functional SETD5 protein, and a second allele from which the SETD5 protein is not produced. In another such embodiment, the subject has a first allele encoding a functional SETD5 protein, and a second allele encoding a nonfunctional SETD5 protein. In another such embodiment, the subject has a first allele encoding a functional SETD5 protein, and a second allele encoding a partially functional SETD5 protein. In any of these embodiments, the antisense oligomer binds to a targeted portion of the NIE containing pre-mRNA transcribed from the second allele, thereby inducing exon skipping of the NMD exon from the pre-mRNA and causing an increase in the level of mature mRNA encoding functional SETD5 protein, and an increase in the expression of the SETD5 protein in the cells of the subject.

In some embodiments, the method is a method of decreasing the expression of the target protein by cells of a subject having a NSAE pre-mRNA encoding the target protein, wherein the subject has a disease caused by an excess amount of activity of the target protein, wherein the excess amount of the target protein is caused by a mutation, and wherein the target gene is SETD5. In some embodiments, the antisense oligomer binds to a targeted portion of the NSAE pre-mRNA transcribed from the allele carrying a mutation, thereby increasing alternate splicing of NSAEs into the pre-mRNA, and causing an decrease in the level of mature mRNA encoding the functional target protein, and an decrease in the expression of the target protein in the cells of the subject. In related embodiments, the method is a method of using an ASO to decrease the expression of a functional protein or functional RNA. In some embodiments, an ASO is used to decrease the expression of the target protein in cells of a subject having a NSAE pre-mRNA encoding the target protein, wherein the subject has an excess in the amount or function of the target protein.

In some embodiments, the method is a method of modulating the expression of the target protein by cells of a subject having a NSAE pre-mRNA encoding the target protein, wherein the subject has a disease caused by a deficient or excess amount of activity of the target protein, wherein the deficient or excess amount of the target protein is caused by a mutation, and wherein the target gene is SETD5. In some embodiments, the antisense oligomer binds to a targeted portion of the NSAE pre-mRNA transcribed from the allele carrying a mutation, thereby modulating alternate splicing of NSAEs into the pre-mRNA, and causing a modulation in the level of mature mRNA encoding the functional target protein, and an modulation in the expression of the target protein in the cells of the subject. In related embodiments, the method is a method of using an ASO to modulate the expression of a functional protein or functional RNA. In some embodiments, an ASO is used to modulate the expression of the target protein in cells of a subject having a NSAE pre-mRNA encoding the target protein, wherein the subject has an abnormality in the amount or function of the target protein.

In some embodiments, the method is a method of increasing the expression of the SETD5 protein by cells of a subject having a NIE containing pre-mRNA encoding the SETD5 protein, wherein the subject has an intellectual disability, or an autism spectrum disease caused by a deficient amount of activity of SETD5 protein, and wherein the deficient amount of the SETD5 protein is caused by autosomal recessive inheritance. In some embodiments, the subject has a disease or condition selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21).

In some embodiments, the method is a method of increasing the expression of the SETD5 protein by cells of a subject having a NIE containing pre-mRNA encoding the SETD5 protein, wherein the subject has an intellectual disability, or an autism spectrum disease caused by a deficient amount of activity of SETD5 protein, and wherein the deficient amount of the SETD5 protein is caused by autosomal dominant inheritance. In some embodiments, the subject has a disease or condition selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21).

In some embodiments, the method is a method of increasing the expression of the SETD5 protein by cells of a subject having a NIE containing pre-mRNA encoding the SETD5 protein, wherein the subject has an intellectual disability, or an autism spectrum disease caused by a deficient amount of activity of SETD5 protein, and wherein the deficient amount of the SETD5 protein is caused by X-linked dominant inheritance. In some embodiments, the subject has a disease or condition selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21).

In related embodiments, the method is a method of using an ASO to increase the expression of a protein or functional RNA. In some embodiments, an ASO may be used to increase the expression of SETD5 protein in cells of a subject having a NIE containing pre-mRNA encoding SETD5 protein, wherein the subject has a deficiency, e.g., an intellectual disability or an autism spectrum disease, in the amount or function of a SETD5 protein. In some embodiments, the subject has a disease or condition selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21).

In some embodiments, the NIE containing pre-mRNA transcript that encodes the protein that is causative of the disease or condition is targeted by the ASOs described herein. In some embodiments, a NIE containing pre-mRNA transcript that encodes a protein that is not causative of the disease is targeted by the ASOs. For example, a disease that is the result of a mutation or deficiency of a first protein in a particular pathway may be ameliorated by targeting a NIE containing pre-mRNA that encodes a second protein, thereby increasing production of the second protein. In some embodiments, the function of the second protein is able to compensate for the mutation or deficiency of the first protein (which is causative of the disease or condition).

In some embodiments, the subject has a disease or condition is associated with a loss-of-function mutation in the target gene or the target protein. In some embodiments, the disease or condition is associated with haploinsufficiency of the target gene. In some embodiments, the subject has a first allele of the target gene encoding a functional protein, and a second allele of the target gene from which the protein is not produced or produced at a reduced level, or a second allele of the target gene encoding a nonfunctional protein or a partially functional protein.

In some embodiments, the subject has a disease or condition associated with an autosomal recessive mutation of a SETD5 gene. In some embodiments, the subject has a first allele encoding from which: (i) the target protein is not produced or produced at a reduced level compared to a wild-type allele; or (ii) the target protein produced is nonfunctional or partially functional compared to a wild-type allele, and a second allele from which: (iii) the target protein is produced at a reduced level compared to a wild-type allele and the target protein produced is at least partially functional compared to a wild-type allele; or (iv) the target protein produced is partially functional compared to a wild-type allele.

In some embodiments, the subject has:
(a) a first mutant allele from which
   (i) the SETD5 protein is produced at a reduced level compared to production from a wild-type allele,
   (ii) the SETD5 protein is produced in a form having reduced function compared to an equivalent wild-type protein, or
   (iii) the SETD5 protein or functional RNA is not produced; and
(b) a second mutant allele from which
   (i) the SETD5 protein is produced at a reduced level compared to production from a wild-type allele,
   (ii) the SETD5 protein is produced in a form having reduced function compared to an equivalent wild-type protein, or
   (iii) the SETD5 protein is not produced, and
      wherein the NIE containing pre-mRNA is transcribed from the first allele and/or the second allele. In these embodiments, the ASO binds to a targeted portion of the NIE containing pre-mRNA transcribed from the first allele or the second allele, thereby inducing exon skipping of the NMD exon from the NIE containing pre-mRNA and causing an increase in the level of mRNA encoding SETD5 protein and an increase in the expression of the target protein or functional RNA in the cells of the subject. In these embodiments, the target protein or functional RNA having an increase in expression level resulting from the exon skipping of the NMD exon from the NIE containing pre-mRNA may be either in a form having reduced function compared to the equivalent wild-type protein (partially functional), or having full function compared to the equivalent wild-type protein (fully functional).

In some embodiments, the level of mRNA encoding SETD5 protein is increased 1.1 to 10-fold, when compared to the amount of mRNA encoding SETD5 protein that is produced in a control cell, e.g., one that is not treated with the antisense oligomer or one that is treated with an antisense oligomer that does not bind to the targeted portion of the SETD5 NIE containing pre-mRNA.

In some embodiments, a subject treated using the methods of the present disclosure expresses a partially functional SETD5 protein from one allele, wherein the partially functional SETD5 protein may be caused by a frameshift mutation, a nonsense mutation, a missense mutation, or a partial gene deletion. In some embodiments, a subject treated using the methods of the disclosure expresses a nonfunctional SETD5 protein from one allele, wherein the nonfunctional SETD5 protein may be caused by a frameshift mutation, a nonsense mutation, a missense mutation, a partial gene deletion, in one allele. In some embodiments, a subject treated using the methods of the disclosure has a SETD5 whole gene deletion, in one allele.

### Exon Inclusion

As used herein, a “non-sense mediated RNA decay alternative exon” (or “NSAE” or “NMD exon”) is an exon created from alternative splicing events that contains a premature stop codon or leads to the introduction of a premature termination codon or other sequences that triggers degradation of the mRNA containing the of the NMD exon. As used herein, a “NIE containing pre-mRNA” is a pre-mRNA transcript that contains at least one NMD-inducing-exon. Alternative or aberrant splicing can result in inclusion of the at least one NMD exon in the mature mRNA transcripts. The terms “mature mRNA,” and “fully-spliced mRNA,” are used interchangeably herein to describe a fully processed mRNA. Inclusion of the at least one NMD exon can be non-productive mRNA and lead to NMD of the mature mRNA. NIE containing mature mRNA may sometimes lead to aberrant protein expression.

In some embodiments, the included NMD exon is the most abundant NMD exon in a population of NIE containing pre-mRNAs transcribed from the gene encoding the target protein in a cell. In some embodiments, the included NMD exon is the most abundant NMD exon in a population of NIE containing pre-mRNAs transcribed from the gene encoding the target protein in a cell, wherein the population of NIE containing pre-mRNAs comprises two or more included NMD exons. In some embodiments, an antisense oligomer targeted to the most abundant NMD exon in the population of NIE containing pre-mRNAs encoding the target protein induces exon skipping of one or two or more NMD exons in the population, including the NMD exon to which the antisense oligomer is targeted or binds. In some embodiments, the targeted region is in a NMD exon that is the most abundant NMD exon in a NIE containing pre-mRNA encoding the SETD5 protein.

The degree of exon inclusion can be expressed as percent exon inclusion, e.g., the percentage of transcripts in which a given NMD exon is included. In brief, percent exon inclusion can be calculated as the percentage of the amount of RNA transcripts with the exon inclusion, over the sum of the average of the amount of RNA transcripts with exon inclusion plus the average of the amount of RNA transcripts with exon exclusion.

In some embodiments, an included NMD exon is an exon that is identified as an included NMD exon based on a determination of at least about 5%, at least about 10%, at least about 15%, at least about 20%, at least about 25%, at least about 30%, at least about 35%, at least about 40%, at least about 45%, or at least about 50%, inclusion. In embodiments, a included NMD exon is an exon that is identified as a included NMD exon based on a determination of about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 15% to about 100%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, or about 25% to about 35%, inclusion.

In some embodiments, contacting cells with an ASO that is complementary to a targeted portion of a SETD5 pre-mRNA transcript results in an increase in the amount of SETD5 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SETD5 protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of SETD5 protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SETD5 pre-mRNA transcript results in an increase in the amount of mRNA encoding SETD5, including the mature mRNA encoding the target protein. In some embodiments, the amount of mRNA encoding SETD5 protein, or the mature mRNA encoding the SETD5 protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding SETD5 protein, or the mature mRNA encoding SETD5 protein produced in the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding SETD5 protein, or the mature mRNA encoding SETD5 protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SETD5 NIE containing pre-mRNA.

In some embodiments, contacting cells with an ASO that is complementary to a targeted portion of a SETD5 pre-mRNA transcript results in a decrease in the amount of SETD5 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SETD5 protein produced by the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of SETD5 protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, the level of mRNA encoding SETD5 protein is decreased 1.1 to 10-fold, when compared to the amount of mRNA encoding SETD5 protein that is produced in a control cell, e.g., one that is not treated with the antisense oligomer or one that is treated with an antisense oligomer that does not bind to the targeted portion of the SETD5 pre-mRNA.

In some embodiments, the level of mRNA encoding SETD5 protein is decreased 1.1 to 10-fold, when compared to the amount of mRNA encoding SETD5 protein that is produced in a control cell, e.g., one that is not treated with the antisense oligomer or one that is treated with an antisense oligomer that does not bind to the targeted portion of the SETD5 pre-mRNA.

In some embodiments of the present invention, a subject can have a mutation in SETD5. A variety of pathogenic variants have been reported to cause SETD5 deficiency, including missense variants, nonsense variants, single- and double-nucleotide insertions and deletions, complex insertion/deletions, and splice site variants. In the presence of this pathogenic variant approximately 2%-5% of transcripts are correctly spliced, allowing for residual enzyme activity. In some embodiments, disease results from loss of function of SETD5 caused by SETD5 pathogenic variants that generate truncated proteins or proteins with altered conformations or reduced activity.

The NIE can be in any length. In some embodiments, the NIE comprises a full sequence of an intron, in which case, it can be referred to as intron retention. In some embodiments, the NIE can be a portion of the intron. In some embodiments, the NIE can be a 5′ end portion of an intron including a 5′ss sequence. In some embodiments, the NIE can be a 3′ end portion of an intron including a 3′ss sequence.

In some embodiments, the NIE can be a portion within an intron without inclusion of a 5′ss sequence. In some embodiments, the NIE can be a portion within an intron without inclusion of a 3′ss sequence. In some embodiments, the NIE can be a portion within an intron without inclusion of either a 5′ss or a 3′ss sequence. In some embodiments, the NIE can be from 5 nucleotides to 10 nucleotides in length, from 10 nucleotides to 15 nucleotides in length, from 15 nucleotides to 20 nucleotides in length, from 20 nucleotides to 25 nucleotides in length, from 25 nucleotides to 30 nucleotides in length, from 30 nucleotides to 35 nucleotides in length, from 35 nucleotides to 40 nucleotides in length, from 40 nucleotides to 45 nucleotides in length, from 45 nucleotides to 50 nucleotides in length, from 50 nucleotides to 55 nucleotides in length, from 55 nucleotides to 60 nucleotides in length, from 60 nucleotides to 65 nucleotides in length, from 65 nucleotides to 70 nucleotides in length, from 70 nucleotides to 75 nucleotides in length, from 75 nucleotides to 80 nucleotides in length, from 80 nucleotides to 85 nucleotides in length, from 85 nucleotides to 90 nucleotides in length, from 90 nucleotides to 95 nucleotides in length, or from 95 nucleotides to 100 nucleotides in length. In some embodiments, the NIE can be at least 10 nucleotides, at least 20 nucleotides, at least 30 nucleotides, at least 40 nucleotides, at least 50 nucleotides, at least 60 nucleoids, at least 70 nucleotides, at least 80 nucleotides in length, at least 90 nucleotides, or at least 100 nucleotides in length. In some embodiments, the NIE can be from 100 to 200 nucleotides in length, from 200 to 300 nucleotides in length, from 300 to 400 nucleotides in length, from 400 to 500 nucleotides in length, from 500 to 600 nucleotides in length, from 600 to 700 nucleotides in length, from 700 to 800 nucleotides in length, from 800 to 900 nucleotides in length, from 900 to 1,000 nucleotides in length. In some embodiments, the NIE may be longer than 1,000 nucleotides in length.

In some embodiments, the pre-mRNA comprises two or more NMD exons. In some embodiments, the pre-mRNA comprises three or more NMD exons. In some embodiments, the pre-mRNA comprises two or more different NMD exons. In some embodiments, the pre-mRNA comprises three or more different NMD exons. In some embodiments, splicing of one or more NMD exons from the pre-mRNA are modulated. In some embodiments, splicing of two or more NMD exons from the pre-mRNA are modulated. In some embodiments, splicing of three or more NMD exons from the pre-mRNA are modulated.

In some embodiments, splicing of one or more NMD exons from the pre-mRNA are increased. In some embodiments, splicing of two or more NMD exons from the pre-mRNA are increased. In some embodiments, splicing of three or more NMD exons from the pre-mRNA are increased. In some embodiments, splicing of one or more NMD exons from the pre-mRNA are decreased. In some embodiments, splicing of two or more NMD exons from the pre-mRNA are decreased. In some embodiments, splicing of three or more NMD exons from the pre-mRNA are decreased.

In some embodiments, splicing of one NMD exon from the pre-mRNA are decreased and other NMD exons from the pre-mRNA are increased. In some embodiments, splicing of one NMD exon from the pre-mRNA are increased and other NMD exons from the pre-mRNA are decreased. In some embodiments, splicing of two NMD exons from the pre-mRNA are decreased and other NMD exons from the pre-mRNA are increased. In some embodiments, splicing of two NMD exons from the pre-mRNA are increased and other NMD exons from the pre-mRNA are decreased. In some embodiments, splicing of three NMD exons from the pre-mRNA are decreased and other NMD exons from the pre-mRNA are increased. In some embodiments, splicing of three NMD exons from the pre-mRNA are increased and other NMD exons from the pre-mRNA are decreased. In some embodiments, splicing of four NMD exons from the pre-mRNA are decreased and other NMD exons from the pre-mRNA are increased. In some embodiments, splicing of four NMD exons from the pre-mRNA are increased and other NMD exons from the pre-mRNA are decreased. In some embodiments, splicing of five NMD exons from the pre-mRNA are decreased and other NMD exons from the pre-mRNA are increased. In some embodiments, splicing of five NMD exons from the pre-mRNA are increased and other NMD exons from the pre-mRNA are decreased. In some embodiments, splicing of some NMD exons from the pre-mRNA are decreased and other NMD exons from the pre-mRNA are increased. In some embodiments, splicing of some NMD exons from the pre-mRNA are increased and other NMD exons from the pre-mRNA are decreased.

In some embodiments, the two or more NMD exons are located in a single intron. In some embodiments, the two or more NMD exons are located in different introns. In some embodiments, the three or more NMD exons are located in a single intron. In some embodiments, the three or more NMD exons are located in different introns.

Inclusion of an NMD exon can lead to a frameshift and the introduction of a premature termination codon (PIC) in the mature mRNA transcript rendering the transcript a target of NMD. Mature mRNA transcript containing NIE can be non-productive mRNA transcript which does not lead to protein expression. The PIC can be present in any position downstream of an NIE. In some embodiments, the PIC can be present in any exon downstream of an NIE. In some embodiments, the PIC can be present within the NIE. For example, inclusion of Exon 5X, exon 3X, or exon 3Y of SETD5 pre-mRNA in an mRNA transcript encoded by the SETD5 gene can induce a PIC in the mRNA transcript. For example, inclusion of exon (GRCh38/hg38: chr3 9429827 9430051), exon (GRCh38/hg38: chr3 9433370 9433562), exon (GRCh38/hg38: chr3 9434591 9434630) or exon (GRCh38/hg38: chr3 9428823 9429009) of SETD5 in an mRNA transcript encoded by SETD5.

### Therapeutic Agents

In some embodiments, the agents as used herein refers to the therapeutic agents. In some embodiments, the therapeutic agents as used herein refers to the agents.

In various embodiments of the present disclosure, compositions and methods comprising a therapeutic agent are provided to modulate protein expression level of SETD5. In some embodiments, provided herein are compositions and methods to modulate alternative splicing of SETD5 pre-mRNA. In some embodiments, provided herein are compositions and methods to induce exon skipping in the splicing of SETD5 pre-mRNA, e.g., to induce skipping of a NMD exon during splicing of SETD5 pre-mRNA. In other embodiments, therapeutic agents may be used to induce the inclusion of an exon in order to decrease the protein expression level.

A therapeutic agent disclosed herein can be a NIE repressor agent. A therapeutic agent may comprise a polynucleic acid polymer. A therapeutic agent disclosed herein can be an alternative splicing repressor agent. In some embodiments, a therapeutic agent may comprise a polynucleic acid polymer. In other embodiments, a therapeutic agent may comprise a small molecule. In other embodiments, a therapeutic agent may comprise a polypeptide. In some embodiments, the therapeutic agent is a nucleic acid binding protein, with or without being complexed with a nucleic acid molecule. In other embodiments, the therapeutic agent is a nucleic acid molecule that encodes for another therapeutic agent. In further embodiments, the therapeutic agent is incorporated into a viral delivery system, such as an adenovirus-associated vector.

According to one aspect of the present disclosure, provided herein is a method of treatment or prevention of a condition or disease associated with a functional SETD5 protein deficiency, comprising administering a NIE repressor agent to a subject to increase levels of functional SETD5 protein, wherein the agent binds to a region of the pre-mRNA transcript to decrease inclusion of the NIE in the mature transcript. For example, provided herein is a method of treatment or prevention of a condition associated with a functional SETD5 protein deficiency, comprising administering a NIE repressor agent to a subject to increase levels of functional SETD5 protein, wherein the agent binds to a region of an intron containing an NIE (e.g., Exon 5X, exon 3Y, or exon 3X of SETD5) of the pre-mRNA transcript or to a NIE-activating regulatory sequence in the same intron. For example, provided herein is a method of treatment or prevention of a condition associated with a functional SETD5 protein deficiency, comprising administering a NIE repressor agent to a subject to increase levels of functional SETD5 protein, wherein the agent binds to a region of an intron containing an NIE (e.g., exon (GRCh38/hg38: chr3 9429827 9430051), exon (GRCh38/hg38: chr3 9433370 9433562), exon (GRCh38/hg38: chr3 9434591 9434630) or exon (GRCh38/hg38: chr3 9428823 9429009) of SETD5) of the pre-mRNA transcript or to a NIE-activating regulatory sequence in the same intron.

According to one aspect of the present disclosure, provided herein is a method of treatment or prevention of a condition associated with a functional-SETD5 protein deficiency, comprising administering an alternative splicing repressor agent to a subject to increase levels of functional SETD5 protein, wherein the agent binds to a region of the pre-mRNA transcript to decrease inclusion of the NSAE in the mature transcript.

Alternatively, for example, provided herein is a method of treatment or prevention of a condition associated with a functional target protein overexpression, comprising administering an alternative splicing repressor agent to a subject to decrease levels of functional target protein, wherein the agent binds to a region of an exon or an intron of the pre-mRNA transcript, wherein the target gene is SETD5.

Where reference is made to reducing NIE inclusion in the mature mRNA, the reduction may be complete, e.g., 100%, or may be partial. The reduction may be clinically significant. The reduction/correction may be relative to the level of NIE inclusion in the subject without treatment, or relative to the amount of NIE inclusion in a population of similar subjects. The reduction/correction may be at least 10% less NIE inclusion relative to the average subject, or the subject prior to treatment. The reduction may be at least 20% less NIE inclusion relative to an average subject, or the subject prior to treatment. The reduction may be at least 40% less NIE inclusion relative to an average subject, or the subject prior to treatment. The reduction may be at least 50% less NIE inclusion relative to an average subject, or the subject prior to treatment. The reduction may be at least 60% less NIE inclusion relative to an average subject, or the subject prior to treatment. The reduction may be at least 80% less NIE inclusion relative to an average subject, or the subject prior to treatment. The reduction may be at least 90% less NIE inclusion relative to an average subject, or the subject prior to treatment.

Where reference is made to increasing active SETD5 protein levels, the increase may be clinically significant. The increase may be relative to the level of active SETD5 protein in the subject without treatment, or relative to the amount of active SETD5 protein in a population of similar subjects. The increase may be at least 10% more active SETD5 protein relative to the average subject, or the subject prior to treatment. The increase may be at least 20% more active SETD5 protein relative to the average subject, or the subject prior to treatment. The increase may be at least 40% more active SETD5 protein relative to the average subject, or the subject prior to treatment. The increase may be at least 50% more active SETD5 protein relative to the average subject, or the subject prior to treatment. The increase may be at least 80% more active SETD5 protein relative to the average subject, or the subject prior to treatment. The increase may be at least 100% more active SETD5 protein relative to the average subject, or the subject prior to treatment. The increase may be at least 200% more active SETD5 protein relative to the average subject, or the subject prior to treatment. The increase may be at least 500% more active SETD5 protein relative to the average subject, or the subject prior to treatment.

Where reference is made to decreasing functional-SETD5 protein levels, the decrease may be clinically significant. The decrease may be relative to the level of functional-SETD5 protein in the subject without treatment, or relative to the amount of functional-SETD5 protein in a population of similar subjects. The decrease may be at least 10% less functional-SETD5 protein relative to the average subject, or the subject prior to treatment. The decrease may be at least 20% less functional-SETD5 protein relative to the average subject, or the subject prior to treatment. The decrease may be at least 40% less functional-SETD5 protein relative to the average subject, or the subject prior to treatment. The decrease may be at least 50% less functional-SETD5 protein relative to the average subject, or the subject prior to treatment. The decrease may be at least 80% less functional-SETD5 protein relative to the average subject, or the subject prior to treatment. The decrease may be at least 100% less functional-SETD5 protein relative to the average subject, or the subject prior to treatment.

In embodiments wherein the NIE repressor agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

In embodiments wherein the alternative splicing repressor agent comprises a polynucleic acid polymer, the polynucleic acid polymer may be about 50 nucleotides in length. In embodiments wherein the alternative splicing modulator agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

The sequence of the polynucleic acid polymer may have 4 or fewer mismatches to a target sequence of the pre-mRNA transcript. The sequence of the polynucleic acid polymer may have 3 or fewer mismatches to a target sequence of the pre-mRNA transcript. The sequence of the polynucleic acid polymer may have 2 or fewer mismatches to a target sequence of the pre-mRNA transcript. The sequence of the polynucleic acid polymer may have 1 or fewer mismatches to a target sequence of the pre-mRNA transcript. The sequence of the polynucleic acid polymer may have no mismatches to a target sequence of the pre-mRNA transcript.

The polynucleic acid polymer may specifically hybridize to a target sequence of the pre-mRNA transcript. For example, the polynucleic acid polymer may have 91%, 92%, 93%, 94%, 95%, 96%, 97%, 98%, 99%, 99.5% or 100% sequence complementarity to a target sequence of the pre-mRNA transcript. The hybridization may be under high stringent hybridization conditions.

The polynucleic acid polymer comprises a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of the sequences listed in Table 4 (e.g., the sequence set forth in any one of SEQ ID NO: 1-331). The polynucleic acid polymer may comprise a sequence with 100% sequence identity to a sequence selected from the group consisting of the sequences listed in Table 4 (e.g., the sequence set forth in any one of SEQ ID NO: 1-331). The polynucleic acid polymer is a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of the sequences listed in Table 4 (e.g., the sequence set forth in any one of SEQ ID NO: 1-331). The polynucleic acid polymer is a sequence with 100% sequence identity to a sequence selected from the group consisting of the sequences listed in Table 4 (e.g., the sequence set forth in any one of SEQ ID NO: 1-331).

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence. References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

### Antisense Oligomers

Provided herein is a composition comprising an antisense oligomer that induces exon skipping by binding to a targeted portion of a SETD5 NIE containing pre-mRNA. As used herein, the terms “ASO” and “antisense oligomer” are used interchangeably and refer to an oligomer such as a polynucleotide, comprising nucleobases that hybridizes to a target nucleic acid (e.g., a SETD5 NIE containing pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art, for example, in PCT Application No. PCT/US2014/054151, published as WO 2015/035091, titled “Reducing Nonsense-Mediated mRNA Decay,” incorporated by reference herein, can be used to practice the methods described herein.

In some embodiments, ASOs “specifically hybridize” to or are “specific” to a target nucleic acid or a targeted portion of a NIE containing pre-mRNA. Typically, such hybridization occurs with a Tₘ substantially greater than 37° C., preferably at least 50° C., and typically between 60° C. to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a target portion of a NIE containing pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5,6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example, in a peptide nucleic acid (PNA), or linking groups including carbamate, amides, and linear and cyclic hydrocarbon groups. In some embodiments, the backbone modification is a phosphothioate linkage. In some embodiments, the backbone modification is a phosphoramidate linkage.

In some embodiments, the stereochemistry at each of the phosphorus internucleotide linkages of the ASO backbone is random. In some embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In some embodiments, an ASO used in the methods of the disclosure, including, but not limited to, any of the ASOs set forth herein in Table 4 (e.g., the sequence set forth in any one of SEQ ID NO: 1-331), comprises an ASO having phosphorus internucleotide linkages that are not random. In some embodiments, a composition used in the methods of the disclosure comprises a pure diastereomeric ASO. In some embodiments, a composition used in the methods of the disclosure comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second-generation antisense oligonucleotides containing chiral phosphorothioate linkages,” Nucleic Acids Research 42(22): 13456-13468, incorporated herein by reference). In some embodiments, an ASO used in the methods of the disclosure, including, but not limited to, any of the ASOs set forth herein in SEQ ID NOs: 1-331, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 1-331, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 1-331, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 1-331, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′-F, 2′-NMA; N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′-F, 2′-MOE, and 2′-NMA. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-O ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

As used herein, “2′-NMA” can mean a —O—CH2-C(═O)—NH—CH3 group in place of the 2′-OH group of a ribosyl sugar moiety. A “2′-NMA sugar moiety” or “2′-NMA moiety” is a sugar moiety with a 2′-O—CH2-C(═O)—NH—CH3 group in place of the 2′-OH group of a ribosyl sugar moiety. Unless otherwise indicated, a 2′-NMA sugar moiety is in the β-D configuration. “NMA” can mean O—N-methyl acetamide. As used herein, “2′-NMA nucleoside” can mean a nucleoside comprising a 2′-NMA sugar moiety.

In some embodiments, each monomer of the ASO is modified in the same way, for example, each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a SETD5 NIE containing pre-mRNA that is downstream (in the 3′ direction) of the 5′ splice site (or 3′ end of the NIE) of the included exon in a SETD5 NIE containing pre-mRNA (e.g., the direction designated by positive numbers relative to the 5′ splice site). In some embodiments, the ASOs are complementary to a targeted portion of the SETD5 NIE containing pre-mRNA that is within the region about +1 to about +500 relative to the 5′ splice site (or 3′ end) of the included exon. In some embodiments, the ASOs may be complementary to a targeted portion of a SETD5 NIE containing pre-mRNA that is within the region between nucleotides +6 and +40,000 relative to the 5′ splice site (or 3′ end) of the included exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20 relative to 5′ splice site (or 3′ end) of the included exon. In some aspects, the ASOs are complementary to a targeted portion that is within the region from about +1 to about +100, from about +100 to about +200, from about +200 to about +300, from about +300 to about +400, or from about +400 to about +500 relative to 5′ splice site (or 3′ end) of the included exon.

In some embodiments, the ASOs are complementary to (and bind to) a targeted portion of a SETD5 NIE containing pre-mRNA that is upstream (in the 5′ direction) of the 5′ splice site (or 3′ end) of the included exon in a SETD5 NIE containing pre-mRNA (e.g., the direction designated by negative numbers relative to the 5′ splice site). In some embodiments, the ASOs are complementary to a targeted portion of the SETD5 NIE containing pre-mRNA that is within the region about −4 to about −270 relative to the 5′ splice site (or 3′end) of the included exon. In some embodiments, the ASOs may be complementary to a targeted portion of a SETD5 NIE containing pre-mRNA that is within the region between nucleotides −1 and −40,000 relative to the 5′ splice site (or 3′ end) of the included exon. In some aspects, the ASOs are complementary to a targeted portion that is within the region about −1 to about −40,000, about −1 to about −30,000, about −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 5′ splice site (or 3′ end) of the included exon.

In some embodiments, the ASOs are complementary to a targeted region of a SETD5 NIE containing pre-mRNA that is upstream (in the 5′ direction) of the 3′ splice site (or 5′ end) of the included exon in a SETD5 NIE containing pre-mRNA (e.g., in the direction designated by negative numbers). In some embodiments, the ASOs are complementary to a targeted portion of the SETD5 NIE containing pre-mRNA that is within the region about −1 to about −500 relative to the 3′ splice site (or 5′ end) of the included exon. In some embodiments, the ASOs are complementary to a targeted portion of the SETD5 NIE containing pre-mRNA that is within the region −1 to −40,000 relative to the 3′ splice site of the included exon. In some aspects, the ASOs are complementary to a targeted portion that is within the region about −1 to about −40,000, about −1 to about −30,000, −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 3′ splice site of the included exon. In some aspects, the ASOs are complementary to a targeted portion that is within the region from about −1 to about −100, from about −100 to about −200, from about −200 to about −300, from about −300 to about −400, or from about −400 to about −500 relative to 3′ splice site of the included exon.

In some embodiments, the ASOs are complementary to a targeted region of a SETD5 NIE containing pre-mRNA that is downstream (in the 3′ direction) of the 3′ splice site (5′ end) of the included exon in a SETD5 NIE containing pre-mRNA (e.g., in the direction designated by positive numbers). In some embodiments, the ASOs are complementary to a targeted portion of the SETD5 NIE containing pre-mRNA that is within the region of about +1 to about +40,000 relative to the 3′ splice site of the included exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20, or about +1 to about +10 relative to 3′ splice site of the included exon.

In some embodiments, the targeted portion of the SETD5 NIE containing pre-mRNA is within the region +100 relative to the 5′ splice site (3′ end) of the included exon to −100 relative to the 3′ splice site (5′ end) of the included exon. In some embodiments, the targeted portion of the SETD5 NIE containing pre-mRNA is within the NIE. In some embodiments, the target portion of the SETD5 NIE containing pre-mRNA comprises a NMD exon and intron boundary.

The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted portion of the NIE containing pre-mRNA are used. In some embodiments, two or more ASOs that are complementary to different targeted portions of the NIE containing pre-mRNA are used.

In some embodiments, the antisense oligonucleotides of the disclosure are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a SETD5 NIE containing pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

In some embodiments, an ASO that targets a pre-mRNA disclosed herein is selected from the group consisting of the sequences listed in Table 4 (e.g., the sequence set forth in any one of SEQ ID NO: 1-331).

### Pharmaceutical Compositions

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present disclosure includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In embodiments, the present disclosure employs a penetration enhancer to effect the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple antisense oligonucleotides. In embodiments, the antisense oligonucleotide is administered in combination with another drug or therapeutic agent.

### Combination Therapies

In some embodiments, provided herein is a composition comprising one or more NSAE-modulating agents. In some embodiments, provided herein is a composition comprising two or more NSAE-modulating agents. In some embodiments, provided herein is a composition comprising one or more ASO complementary to a targeted region of SETD5 pre-mRNA. In some embodiments, provided herein is a composition comprising two or more ASO complementary to a targeted region of SETD5 pre-mRNA. In some embodiments, provided herein is a composition comprising one or more ASO complementary to a same targeted region of SETD5 pre-mRNA. In some embodiments, provided herein is a composition comprising two or more ASO complementary to a same targeted region of SETD5 pre-mRNA. In some embodiments, provided herein is a composition comprising one or more ASO complementary to different targeted regions of SETD5 pre-mRNA. In some embodiments, provided herein is a composition comprising two or more ASO complementary to different targeted regions of SETD5 pre-mRNA. In some embodiments, provided herein is a composition comprising one or more ASOs of Table 4 (e.g., the sequence set forth in any one of SEQ ID NO: 1-331). In some embodiments, provided herein is a composition comprising two and more ASOs of in Table 4 (e.g., the sequence set forth in any one of SEQ ID NO: 1-331).

In some embodiments, the ASOs disclosed in the present disclosure can be used in combination with one or more additional therapeutic agents. In some embodiments, the one or more additional therapeutic agents can comprise a small molecule. For example, the one or more additional therapeutic agents can comprise a small molecule described in WO2016128343A1, WO2017053982A1, WO2016196386A1, WO201428459A1, WO201524876A2, WO2013119916A2, and WO2014209841A2, which are incorporated by reference herein in their entirety. In some embodiments, the one or more additional therapeutic agents comprise an ASO that can be used to correct intron retention.

### Treatment of Subjects

Any of the compositions provided herein may be administered to an individual. “Individual” may be used interchangeably with “subject” or “patient.” An individual may be a mammal, for example, a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In embodiments, a fetus is treated in utero, e.g., by administering the ASO composition to the fetus directly or indirectly (e.g., via the mother).

Suitable routes for administration of ASOs of the present disclosure may vary depending on cell type to which delivery of the ASOs is desired. The ASOs of the present disclosure may be administered to patients parenterally, for example, by intrathecal injection, intracerebroventricular injection, intraperitoneal injection, intramuscular injection, subcutaneous injection, or intravenous injection.

In embodiments, the antisense oligonucleotide is administered with one or more agents capable of promoting penetration of the subject antisense oligonucleotide across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the antisense oligonucleotides are linked or conjugated with agents that provide desirable pharmaceutical or pharmacodynamic properties. In embodiments, the antisense oligonucleotide is coupled to a substance, known in the art to promote penetration or transport across the blood-brain barrier, e.g., an antibody to the transferrin receptor. In embodiments, the antisense oligonucleotide is linked with a viral vector, e.g., to render the antisense compound more effective or increase transport across the blood-brain barrier. In embodiments, osmotic blood brain barrier disruption is assisted by infusion of sugars, e.g., meso-erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 9,193,969, “Compositions and methods for selective delivery of oligonucleotide molecules to specific neuron types,” U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each incorporated herein by reference.

In some embodiments, an ASO of the disclosure is coupled to a dopamine reuptake inhibitor (DRI), a selective serotonin reuptake inhibitor (SSRI), a noradrenaline reuptake inhibitor (NRI), a norepinephrine-dopamine reuptake inhibitor (NDRI), and a serotonin-norepinephrine-dopamine reuptake inhibitor (SNDRI), using methods described in, e.g., U.S. Pat. No. 9,193,969, incorporated herein by reference.

In some embodiments, subjects treated using the methods and compositions are evaluated for improvement in condition using any methods known and described in the art.

In some cases, a therapeutic agent comprises a modified snRNA, such as a modified human snRNA. In some cases, a therapeutic agent comprises a vector, such as a viral vector, that encodes a modified snRNA. In some embodiments, the modified snRNA is a modified U1 snRNA (see, e.g., Alanis et al., Human Molecular Genetics, 2012, Vol. 21, No. 11 2389-2398). In some embodiments, the modified snRNA is a modified U7 snRNA (see, e.g., Gadgil et al., J Gene Med. 2021; 23:e3321). Modified U7 snRNAs can be made by any method known in the art including the methods described in Meyer, K.; Schümperli, Daniel (2012), Antisense Derivatives of U7 Small Nuclear RNA as Modulators of Pre-mRNA Splicing. In: Stamm, Stefan; Smith, Christopher W. J.; Lührmann, Reinhard (eds.) Alternative pre-mRNA Splicing: Theory and Protocols (pp. 481-494), Chichester: John Wiley & Sons 10.1002/9783527636778.ch45, incorporated by reference herein in its entirety. In some embodiments, a modified U7 (smOPT) does not compete with WT U7 (Stefanovic et al., 1995).

In some embodiments, the modified snRNA comprises an smOPT modification. For example, the modified snRNA can comprise a sequence AAUUUUUGGAG (SEQ ID NO: 409). For example, the sequence AAUUUUUGGAG (SEQ ID NO: 409) can replace a sequence AAUUUGUCUAG (SEQ ID NO: 510) in a wild-type U7 snRNA to generate the modified U& snRNA (smOPT). In some embodiments, a smOPT modification of a U7 snRNA renders the particle functionally inactive in histone pre-mRNA processing (Stefanovic et al., 1995). In some embodiments, a modified U7 (smOPT) is expressed stably in the nucleus and at higher levels than WT U7 (Stefanovic et al., 1995). In some embodiments, the snRNA comprises a U1 snRNP-targeted sequence. In some embodiments, the snRNA comprises a U7 snRNP-targeted sequence. In some embodiments, the snRNA comprises a modified U7 snRNP-targeted sequence and wherein the modified U7 snRNP-targeted sequence comprises smOPT. In some embodiments, the modified snRNA has been modified to comprise a single-stranded nucleotide sequence that hybridizes to a SETD5 pre-mRNA. In some embodiments, the modified snRNA has been modified to comprise a single-stranded nucleotide sequence that hybridizes to a SETD5 mRNA. Exemplary sequences of mouse U7 vector that can be used in the subject compositions and methods include those listed in Table 5. In some embodiments, the antisense oligomer sequence in a U1 or U7 vector disclosed herein has a sequence at least 75%, at least 80%, at least 85%, at least 90%, at least 95%, at least 98%, at least 99%, or 100% identical to a sequence listed in Table 4 (e.g., the sequence set forth in any one of SEQ ID NO: 1-331).

In some embodiments, the modified snRNA has been modified to comprise a single-stranded nucleotide sequence that hybridizes to a target region of a SETD5 pre-mRNA or a processed SETD5 mRNA, such as a target region of a SETD5 pre-mRNA that modulates exclusion of an NMD exon, a target region of a SETD5 pre-mRNA that modulates exclusion of a first exon that comprises a translational start site. In some embodiments, the modified snRNA has been modified to comprise a single-stranded nucleotide sequence that comprises one or two or more sequences of the ASOs disclosed herein. In some embodiments, the modified snRNA has been modified to comprise a single-stranded nucleotide sequence that hybridizes to sequence of a SETD5 pre-mRNA with a mutation, such as a SETD5 NMD exon-containing pre-mRNA with a mutation, or a SETD5 pre-mRNA with a mutation containing a first exon that comprises a translational start site. In some embodiments, the modified snRNA has been modified to comprise a single-stranded nucleotide sequence that comprises two or more sequences that hybridize to two or more target regions of a SETD5 NMD exon-containing pre-mRNA. For example, a modified snRNA can be modified to comprise a single-stranded nucleotide sequence that hybridizes to at least 8 contiguous nucleic acids of a SETD5 NMD exon-containing pre-mRNA. In some embodiments, the modified snRNA has been modified to comprise a single-stranded nucleotide sequence that hybridizes to any of the target regions of a SETD5 NMD exon-containing pre-mRNA disclosed herein. In some embodiments, the modified snRNA has been modified to comprise a single-stranded nucleotide sequence that comprises two or more sequences that hybridize to two or more target regions of a SETD5 NMD exon-containing pre-mRNA. For example, a modified snRNA can be modified to comprise a single-stranded nucleotide sequence that hybridizes to one or two or more sequences of an intron containing an NMD exon (e.g., Exon 5X of SETD5) of the pre-mRNA transcript or to a NMD exon-activating regulatory sequence in the same intron. For example, a modified snRNA can be modified to comprise a single-stranded nucleotide sequence that hybridizes to a region within an NMD exon or upstream or downstream of an NMD exon (e.g., Exon 5X of SETD5). In some embodiments, the modified snRNA has a 5′ region that has been modified to comprise a single-stranded nucleotide sequence that hybridizes to a SETD5 NMD exon-containing pre-mRNA. In some embodiments, the modified snRNA has a 3′ region that has been modified to comprise a single-stranded nucleotide sequence that hybridizes to a SETD5 NMD exon-containing pre-mRNA.

In some embodiments, the modified snRNA has been modified to comprise a single-stranded nucleotide sequence that hybridizes to a target region of a SETD5 pre-mRNA that modulates exclusion of an NMD exon. For example, a modified snRNA can be modified to comprise a single-stranded nucleotide sequence that hybridizes to a region that overlaps with an NMD exon and an intron upstream of the NMD exon (e.g., Exon 5X of SETD5). For example, a modified snRNA can be modified to comprise a single-stranded nucleotide sequence that hybridizes to a region that overlaps with an NMD exon and an intron downstream of the NMD exon (e.g., Exon 5X of SETD5). For example, a modified snRNA can be modified to comprise a single-stranded nucleotide sequence that is complementary to an intron sequence that is downstream of an NMD exon (e.g., Exon 5X of SETD5). For example, a modified snRNA can be modified to comprise a single-stranded nucleotide sequence that is complementary to a 3′ splice site of an intron sequence that is downstream of an NMD exon (e.g., Exon 5X of SETD5). For example, a modified snRNA can be modified to comprise a single-stranded nucleotide sequence that is complementary to a 5′ splice site of an intron sequence that is downstream of an NMD exon (e.g., Exon 5X of SETD5). For example, a modified snRNA can be modified to comprise a single-stranded nucleotide sequence that is complementary to an intron sequence that is upstream of an NMD exon (e.g., Exon 5X of SETD5). For example, a modified snRNA can be modified to comprise a single-stranded nucleotide sequence that is complementary to a splice site of an intron sequence that is upstream of an NMD exon (e.g., Exon 5X of SETD5). For example, a modified snRNA can be modified to comprise a single-stranded nucleotide sequence that is complementary to a 3′ splice site of an intron sequence that is upstream of an NMD exon (e.g., Exon 5X of SETD5). For example, a modified snRNA can be modified to comprise a single-stranded nucleotide sequence that is complementary to a 5′ splice site of an intron sequence that is upstream of an NMD exon (e.g., Exon 5X of SETD5).

In some embodiments, the modified snRNA has been modified to comprise a single-stranded nucleotide sequence that hybridizes to a target region of a SETD5 pre-mRNA that modulates exclusion of a first exon that comprises a translational start site, such as an NMD-exon that comprises a translational start site. For example, a modified snRNA can be modified to comprise a single-stranded nucleotide sequence that hybridizes to a region that is whin an intron containing a first NMD-exon that comprises a translational start site. For example, a modified snRNA can be modified to comprise a single-stranded nucleotide sequence that hybridizes to a region that is upstream of a first exon that comprises a translational start site, such a region within an intron upstream of a first exon that comprises a translational start site. For example, a modified snRNA can be modified to comprise a single-stranded nucleotide sequence that hybridizes to a region that is downstream of a first exon that comprises a translational start site, such a region within an intron downstream of a first exon that comprises a translational start site. For example, a modified snRNA can be modified to comprise a single-stranded nucleotide sequence that hybridizes to a region that is within of a first exon that comprises a translational start site. For example, a modified snRNA can be modified to comprise a single-stranded nucleotide sequence that hybridizes to a region that is overlaps with a splice site of a first exon that comprises a translational start site. For example, a modified snRNA can be modified to comprise a single-stranded nucleotide sequence that hybridizes to a region that is overlaps with a 3′ splice site of a first exon that comprises a translational start site. For example, a modified snRNA can be modified to comprise a single-stranded nucleotide sequence that hybridizes to a region that is overlaps with a 5′ splice site of a first exon that comprises a translational start site.

Methods of Identifying Additional ASOs that Induce Exon Skipping

Also within the scope of the present disclosure are methods for identifying or determining ASOs that induce exon skipping of a SETD5 NIE containing pre-mRNA. For example, a method can comprise identifying or determining ASOs that induce NMD exon skipping of a SETD5 NIE containing pre-mRNA. ASOs that specifically hybridize to different nucleotides within the target region of the pre-mRNA may be screened to identify or determine ASOs that improve the rate and/or extent of splicing of the target intron. In some embodiments, the ASO may block or interfere with the binding site(s) of a splicing repressor(s)/silencer. Any method known in the art may be used to identify (determine) an ASO that when hybridized to the target region of the exon results in the desired effect (e.g., NMD exon skipping, protein or functional RNA production). These methods also can be used for identifying ASOs that induce exon skipping of the included exon by binding to a targeted region in an intron flanking the included exon, or in a non-included exon. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ splice site of the included exon (e.g., a portion of sequence of the exon located upstream of the target/included exon) to approximately 100 nucleotides downstream of the 3′ splice site of the target/included exon and/or from approximately 100 nucleotides upstream of the 5′ splice site of the included exon to approximately 100 nucleotides downstream of the 5′ splice site of the target/included exon (e.g., a portion of sequence of the exon located downstream of the target/included exon). For example, a first ASO of 15 nucleotides in length may be designed to specifically hybridize to nucleotides +6 to +20 relative to the 3′ splice site of the target/included exon. A second ASO may be designed to specifically hybridize to nucleotides +11 to +25 relative to the 3′ splice site of the target/included exon. ASOs are designed as such spanning the target region of the pre-mRNA. In embodiments, the ASOs can be tiled more closely, e.g., every 1, 2, 3, or 4 nucleotides. Further, the ASOs can be tiled from 100 nucleotides downstream of the 5′ splice site, to 100 nucleotides upstream of the 3′ splice site. In some embodiments, the ASOs can be tiled from about 1,160 nucleotides upstream of the 3′ splice site, to about 500 nucleotides downstream of the 5′ splice site. In some embodiments, the ASOs can be tiled from about 500 nucleotides upstream of the 3′ splice site, to about 1,920 nucleotides downstream of the 3′ splice site.

One or more ASOs, or a control ASO (an ASO with a scrambled sequence, sequence that is not expected to hybridize to the target region) are delivered, for example, by transfection, into a disease-relevant cell line that expresses the target pre-mRNA (e.g., a NIE containing pre-mRNA described herein). The exon skipping effects of each of the ASOs may be assessed by any method known in the art, for example, by reverse transcriptase (RT)-PCR using primers that span the splice junction, as described in Example 4. A reduction or absence of a longer RT-PCR product produced using the primers spanning the region containing the included exon (e.g., including the flanking exons of the NIE) in ASO-treated cells as compared to in control ASO-treated cells indicates that splicing of the target NIE has been enhanced. In some embodiments, the exon skipping efficiency (or the splicing efficiency to splice the intron containing the NIE), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in exon skipping (or enhanced splicing of NIE).

Regions defined by ASOs that promote splicing of the target intron are explored in greater detail by means of an ASO “micro-walk”, involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the NIE, as described herein (see, e.g., Example 4). A reduction or absence of a longer RT-PCR product produced using the primers spanning the NIE in ASO-treated cells as compared to in control ASO-treated cells indicates that exon skipping (or splicing of the target intron containing an NIE) has been enhanced. In some embodiments, the exon skipping efficiency (or the splicing efficiency to splice the intron containing the NIE), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

ASOs that when hybridized to a region of a pre-mRNA result in exon skipping (or enhanced splicing of the intron containing a NIE) and increased protein production may be tested in vivo using animal models, for example, transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intrathecal injection, intracerebroventricular injection, intraperitoneal injection, intramuscular injection, subcutaneous injection, or intravenous injection. Following administration, the cells, tissues, and/or organs of the model animals may be assessed to determine the effect of the ASO treatment by, for example, evaluating splicing (e.g., efficiency, rate, extent) and protein production by methods known in the art and described herein. The animal models may also be any phenotypic or behavioral indication of the disease or disease severity.

Also within the scope of the present disclosure is a method to identify or validate an NMD-inducing exon in the presence of an NMD inhibitor, for example, cycloheximide. An exemplary method is provided in Example 2.

TABLE 1
List of exemplary target gene sequences

| Gene | Gene | | | |
|---|---|---|---|---|
| Symbol | ID No. | Disease | OMIM | Genetics |
| SETD5 | 55209 | intellectual disability, | 615743 | Haploinsufficient, |
| | | autism spectrum disorder | | Loss-of-function |

Lengthy table referenced here
US20260035694A1-20260205-T00001
Please refer to the end of the specification for access instructions.

Lengthy table referenced here
US20260035694A1-20260205-T00002
Please refer to the end of the specification for access instructions.

Lengthy table referenced here
US20260035694A1-20260205-T00003
Please refer to the end of the specification for access instructions.

Lengthy table referenced here
US20260035694A1-20260205-T00004
Please refer to the end of the specification for access instructions.

Lengthy table referenced here
US20260035694A1-20260205-T00005
Please refer to the end of the specification for access instructions.

### EXAMPLES

The present disclosure will be more specifically illustrated by the following Examples. However, it should be understood that the present disclosure is not limited by these examples in any manner.

### Example 1: Identification of NMD-Inducing Exon Inclusion Events in Transcripts by RNAseq Using Next-Generation Sequencing

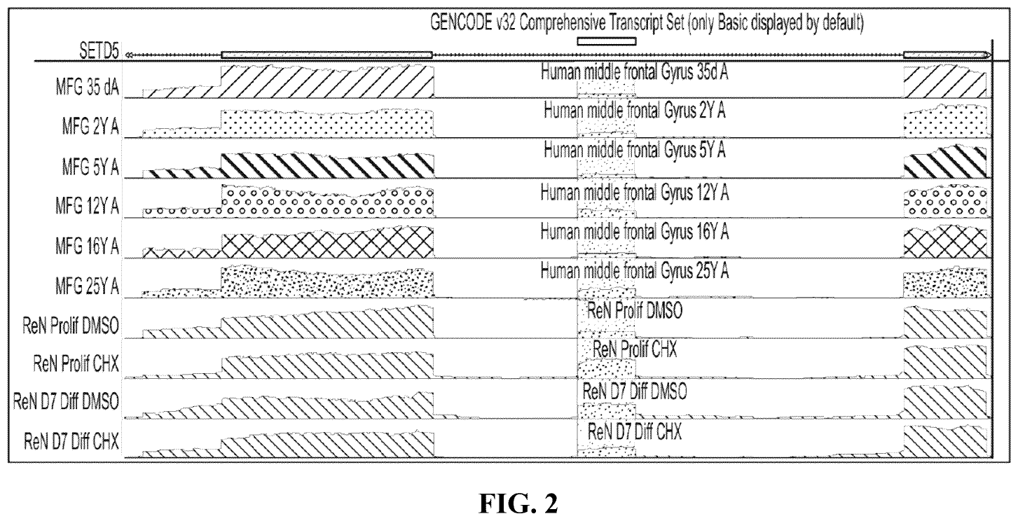

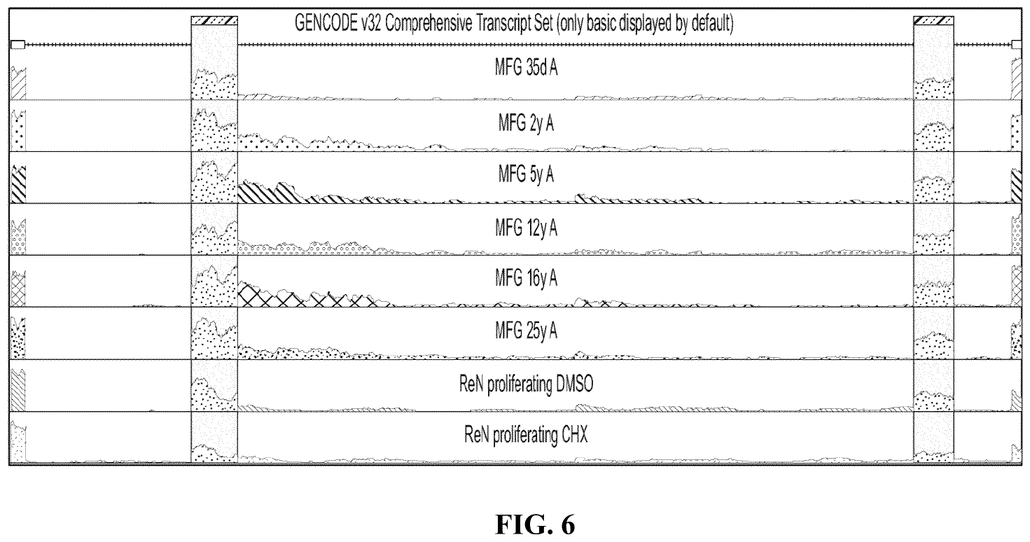

Whole transcriptome shotgun sequencing is carried out using next-generation sequencing to reveal a snapshot of transcripts produced by the genes described herein to identify NIE inclusion events. For this purpose, polyA+RNA from nuclear and cytoplasmic fractions of human cells are isolated and cDNA libraries are constructed using Illumina's TruSeq Stranded mRNA library prep kit. The libraries are pair-end sequenced, resulting in 100-nucleotide reads that are mapped to the human genome (Grch38/hg38 assembly). FIGS. 2 and 6 depict identification of different exemplary nonsense-mediated mRNA decay (NMD)-inducing exons in various genes.

Exemplary genes are summarized in Table 1. The sequence for each intron is summarized in Table 2.

### Example 2: Confirmation of NIE Via Cycloheximide Treatment

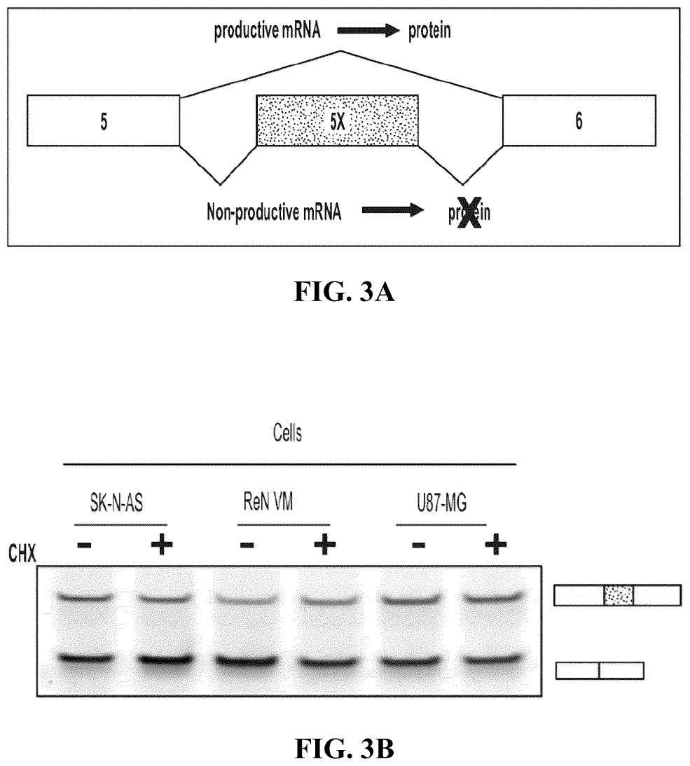

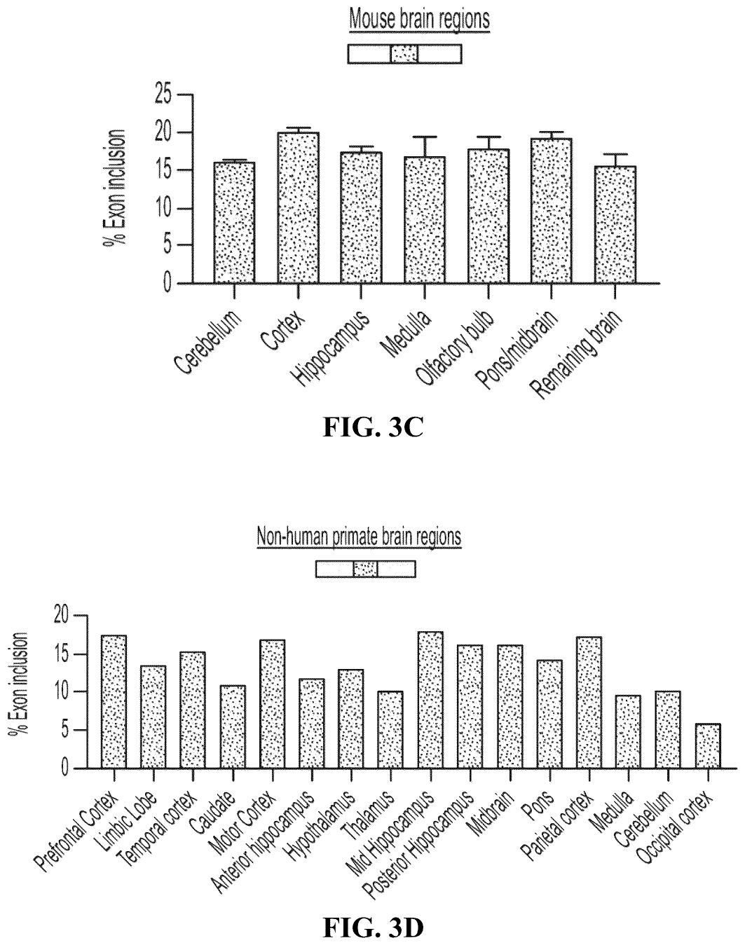

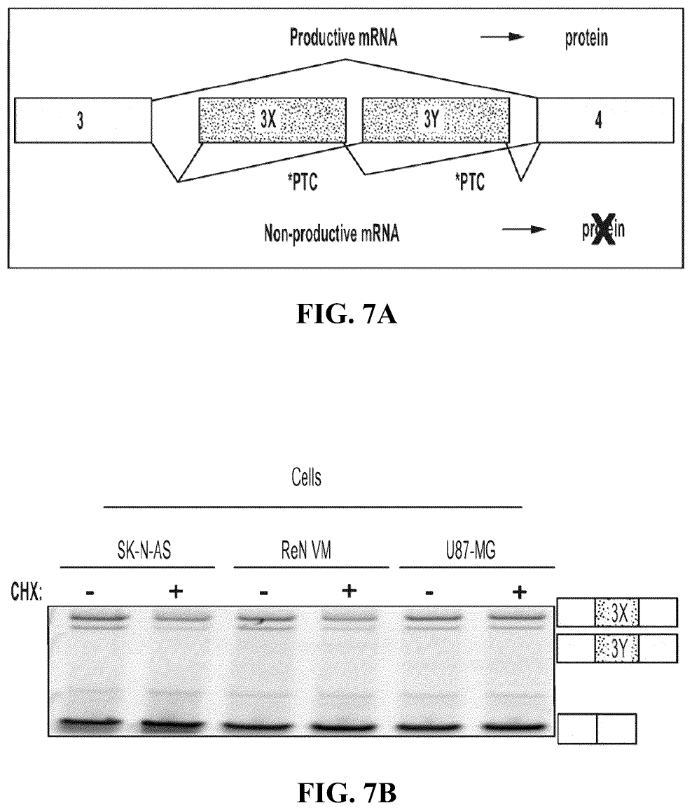

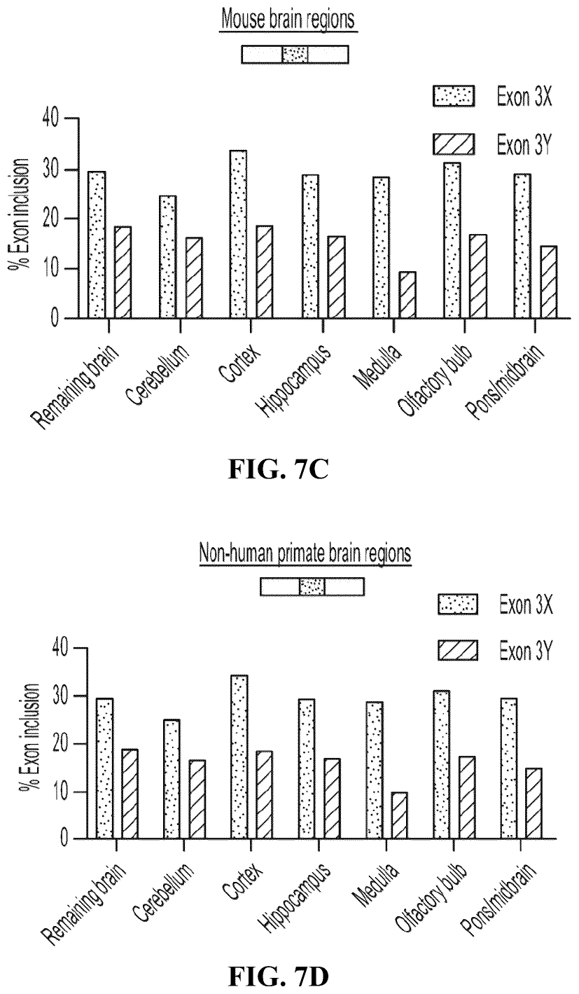

RT-PCR analysis using cytoplasmic RNA from DMSO-treated or cycloheximide-treated SK-N-AS (neuroblastoma), ReNcell VM cells (neural progenitor cells), or U87-MG (likely glioblastoma) cells and primers in exons can confirm the presence of a band corresponding to an NMD-inducing exon. The identity of the product is confirmed by sequencing. Densitometry analysis of the bands is performed to calculate the percentage of NMD exon inclusion of total transcript. Treatment of cells with cycloheximide to inhibit NMD can lead to an increase of the product corresponding to the NMD-inducing exon in the cytoplasmic fraction. FIGS. 3B and 7B depict confirmation of exemplary NIE exons in various gene transcripts using cycloheximide treatment, respectively.

FIGS. 3C and 7C show quantification of the RT-PCR products using RNA from various 2-month-old mouse brain regions plotted as a percentage of exon inclusion isoform (Exon inc/(Exon inc +productive mRNA)*100).

FIGS. 3D and 7D show quantification of the RT-PCR products using RNA from various non-human primate brain regions plotted as a percentage of exon inclusion isoform (Exon inc/(Exon inc +productive mRNA)*100).

### Example 3: NMD Exon Region ASO Walk

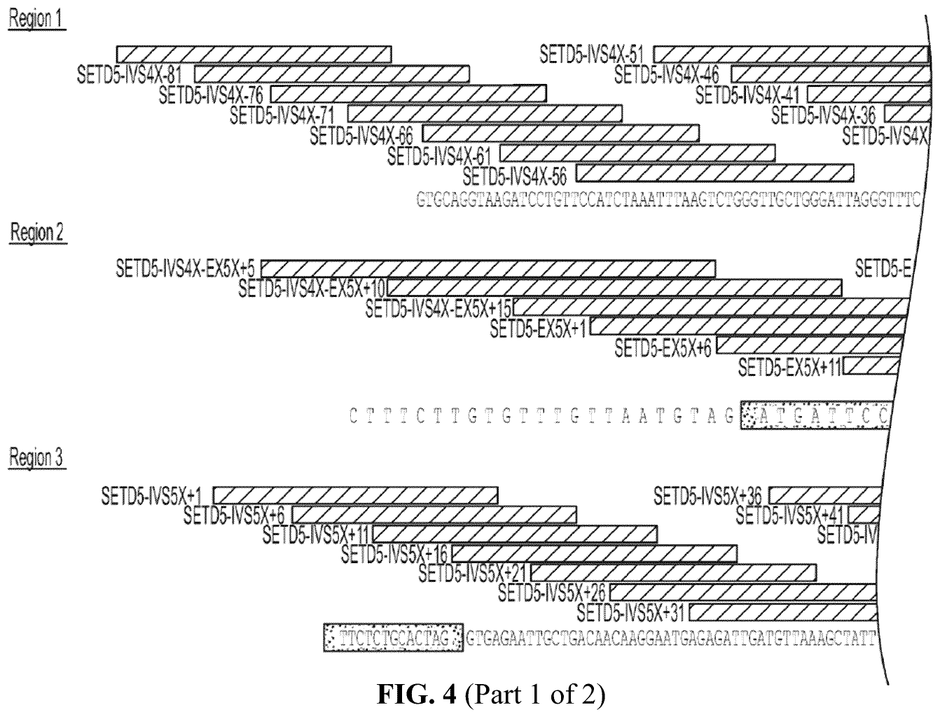

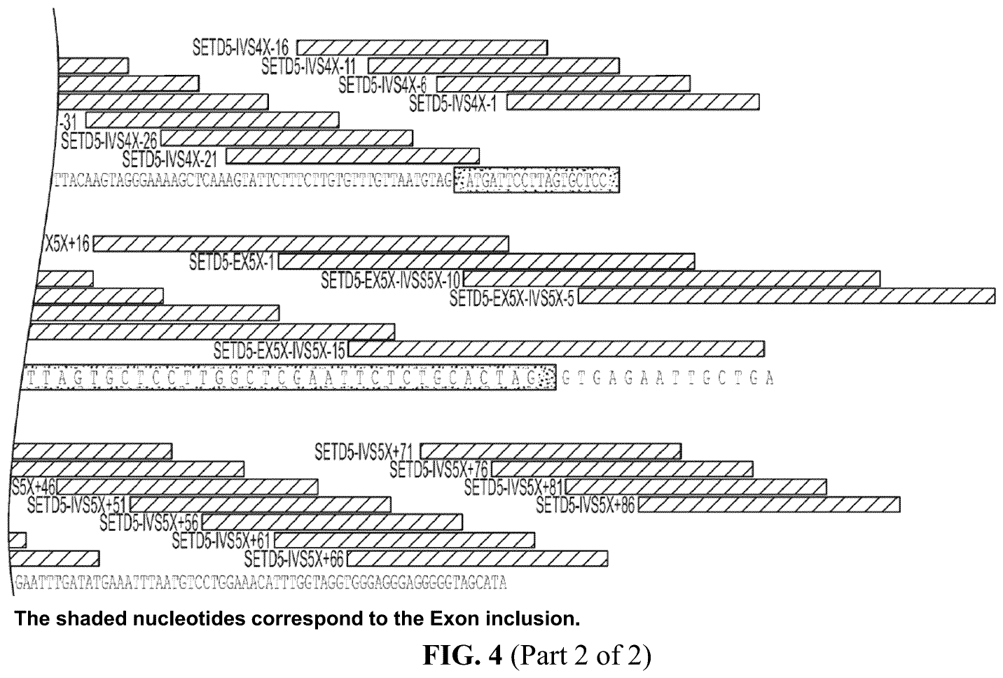

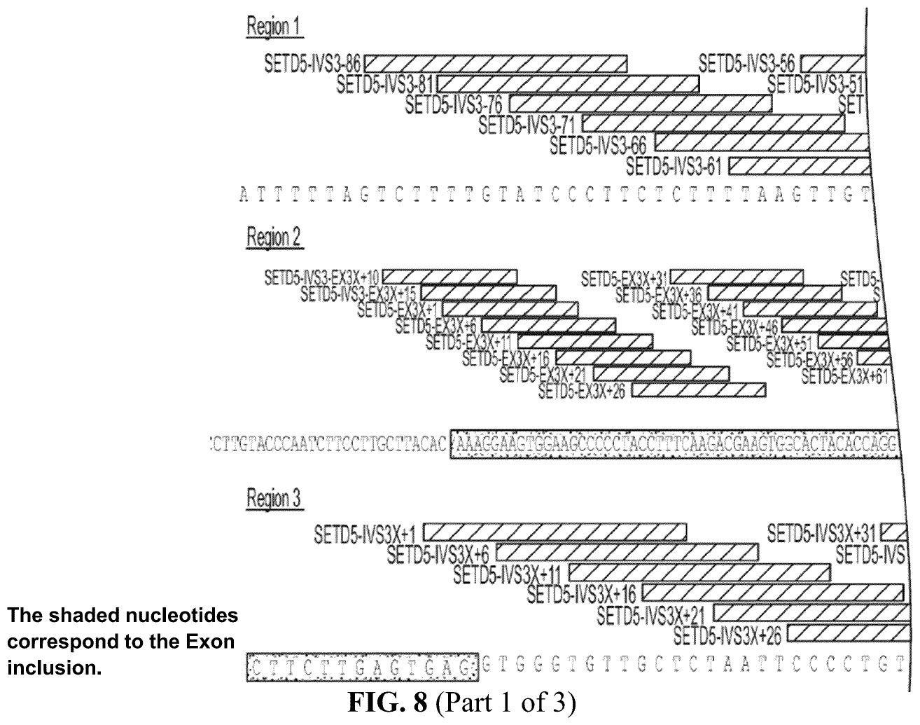

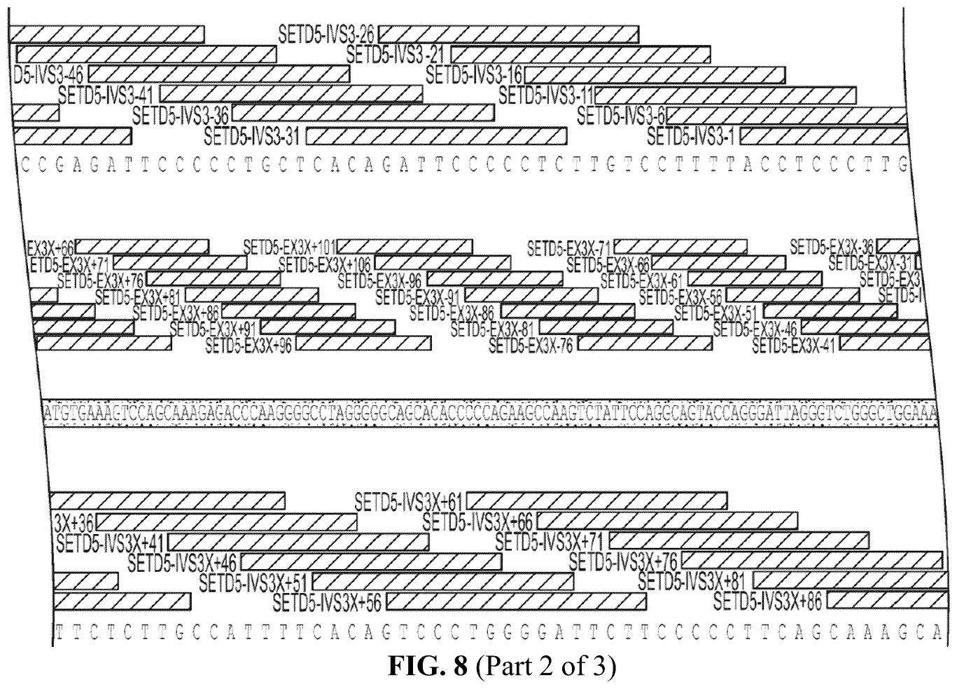

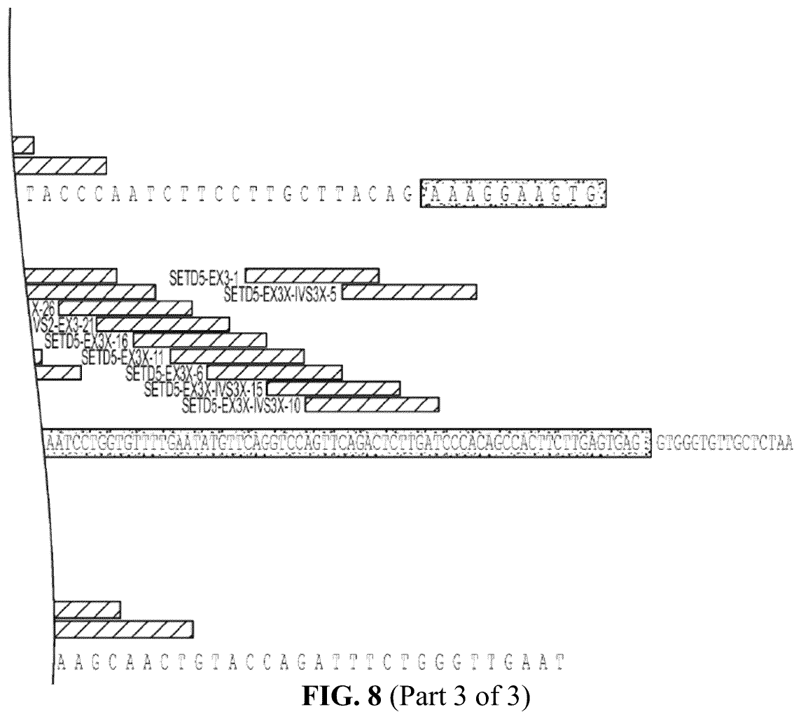

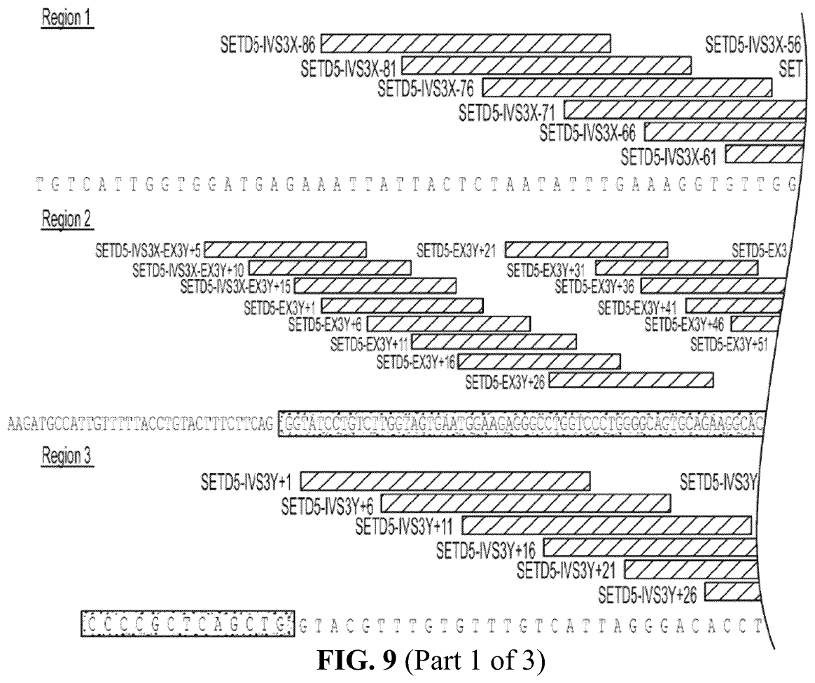

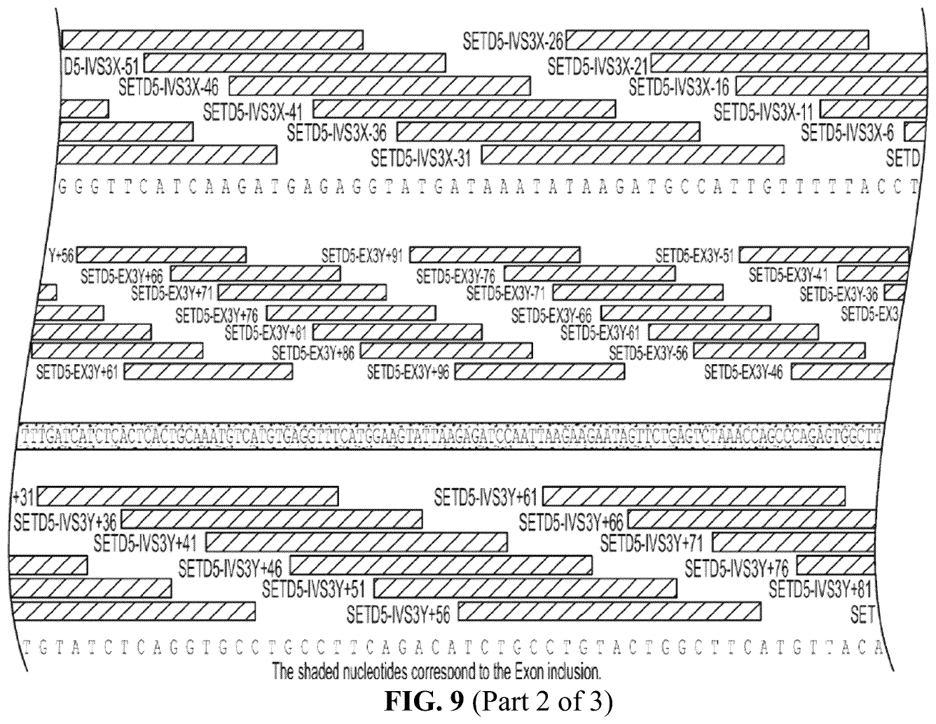

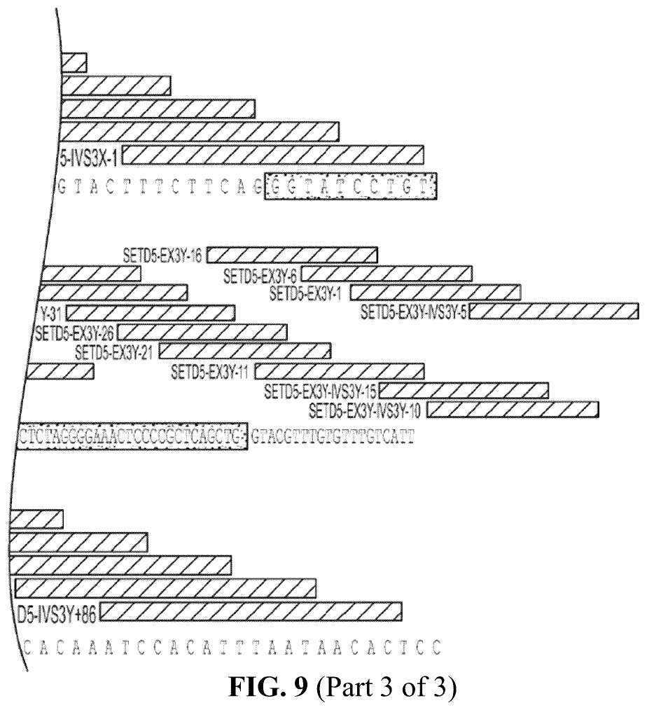

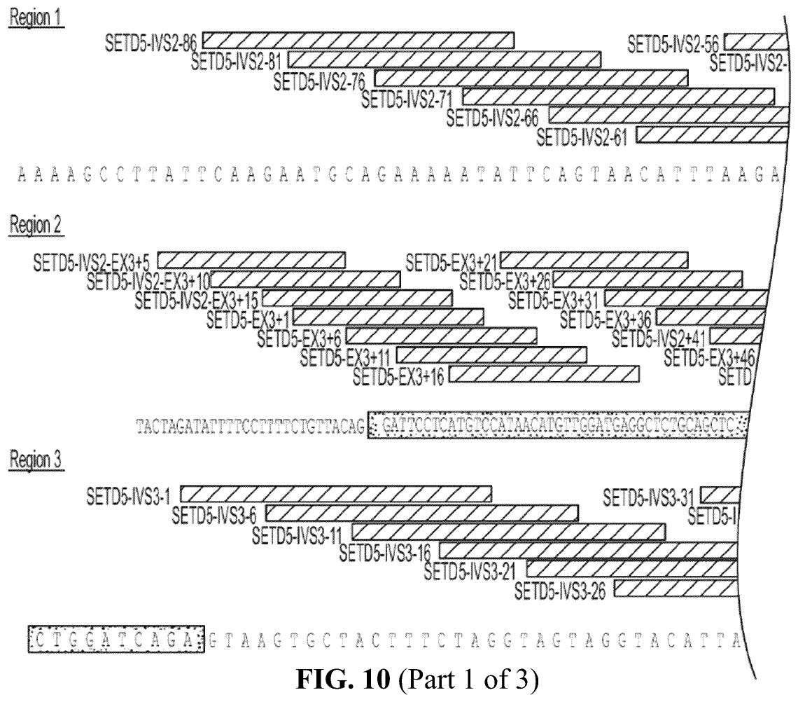

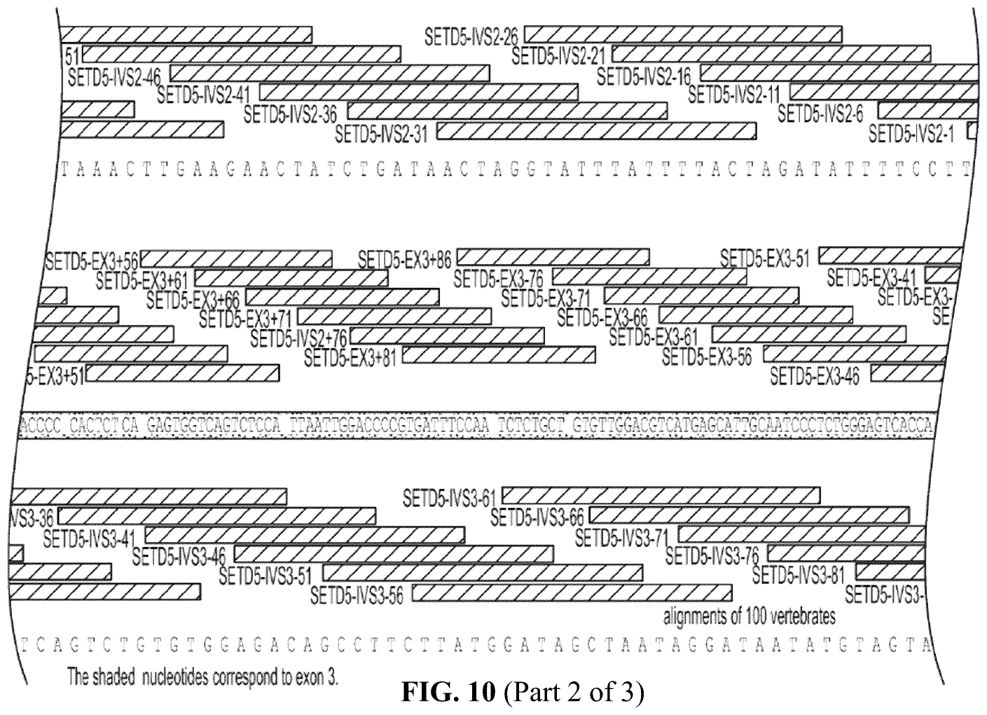

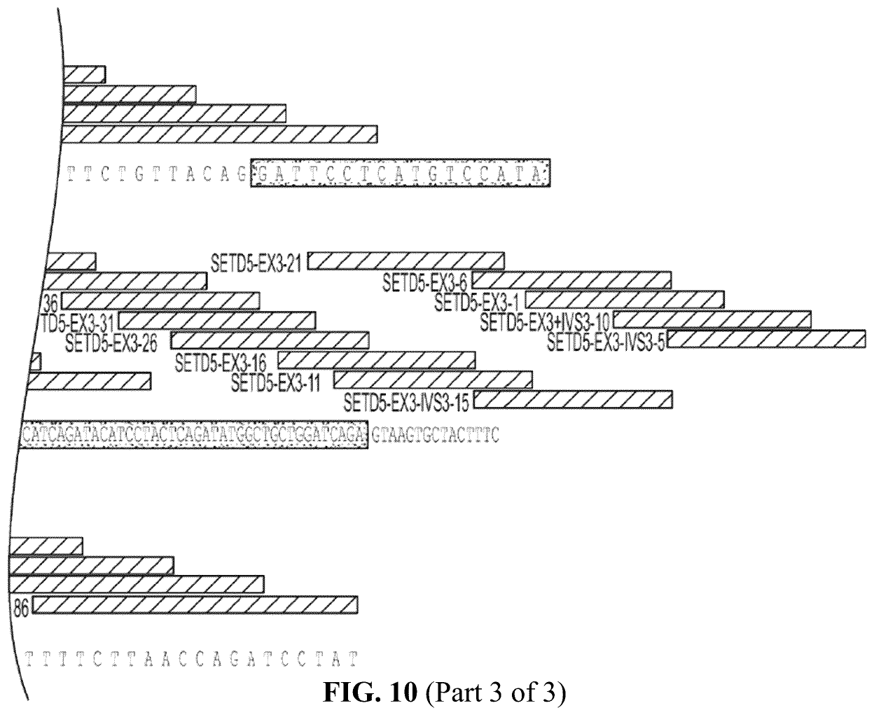

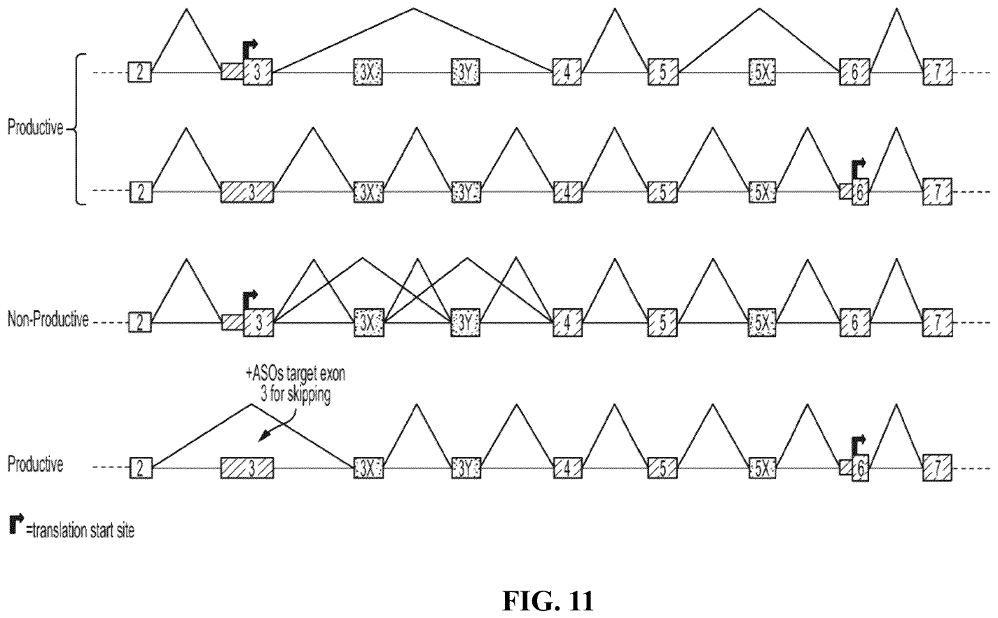

An ASO walk is performed for NMD exon region targeting sequences immediately upstream of the 3′ splice site, across the 3′splice site, the NMD exon, across the 5′ splice site, and downstream of the 5′ splice site using ASOs. ASOs are designed to cover these regions by shifting 5 nucleotides at a time. FIGS. 4, 8, and 9 depict ASO walks for various exemplary NIE exon regions, respectively.

### Example 4: NMD Exon Region ASO Walk Evaluated by RT-PCR

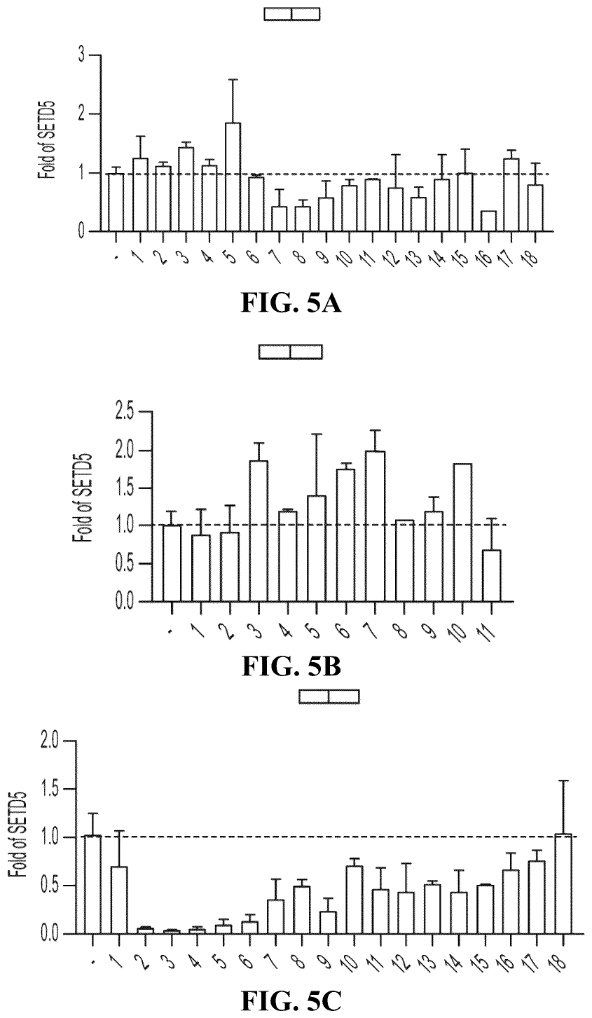

ASO walk sequences can be evaluated by, for example, RT-PCR. PAGE can be used to show SYBR safe-stained RT-PCR products of ReNcell VM cells treated with a ASO targeting the NMD exon regions as described herein at 2 μM concentration in human cells by gymnotic uptake. Products corresponding to NMD exon inclusion and full-length are quantified and the percentage of NMD exon inclusion is plotted; full-length products can be normalized to an RPL32 internal control and fold-change relative to the control can be plotted. FIGS. 5A, 5B, and 5C depict evaluation via RT-PCR of various exemplary ASO walks along exemplary NIE exon regions, respectively.

### Example 5: NMD Exon Region ASO Walk Evaluated by RT-qPCR

SYBR green RT-qPCR amplification results normalized to RPL32, can be obtained using the same ASO uptake experiment that can be evaluated by SYBR-safe RT-PCR, and can be plotted as fold change relative to Sham to confirm SYBR-safe RT-PCR results. FIGS. 5A, 5B, and 5C depict evaluation via RT-qPCR of various exemplary ASO walks along exemplary NIE exon regions, respectively.

FIG. 5A depicts TaqMan qPCR using RNA from ReNcell VM cells evaluated 24 hours after nucleofection with 2 μM ASOs from region 1 depicted in FIG. 4. TaqMan probe spans the Exons 5 and 6 junction (based on the transcript NM_001080517) and measures productive mRNA.

FIG. 5B depicts TaqMan qPCR using RNA from ReNcell VM cells evaluated 24 hours after nucleofection with 2 μM ASOs from region 2 depicted in FIG. 4. TaqMan probe spans the Exons 5 and 6 junction (based on the transcript NM_001080517) and measures productive mRNA.

FIG. 5C depicts TaqMan qPCR using RNA from ReNcell VM cells evaluated 24 hours after nucleofection with 2 μM ASOs from region 3 depicted in FIG. 4. TaqMan probe spans the Exons 5 and 6 junction (based on the transcript NM_001080517) and measures productive mRNA.

### Example 6: Dose-Dependent Effect of Selected ASO in CXH-Treated Cells

PAGE can be used to show SYBR safe-stained RT-PCR products of mock-treated (Sham, RNAiMAX alone), or treatment with ASOs targeting NMD exons at 30 nM, 80 nM, and 200 nM concentrations in mouse or human cells by RNAiMAX transfection. Products corresponding to NMD exon inclusion and full-length are quantified and the percentage of NMD exon inclusion can be plotted. The full-length products can also be normalized to HPRT internal control, and fold-change relative to Sham can be plotted.

### Example 7: Intracerebroventricular (ICV) Injection of Selected ASOs

PAGEs of SYBR safe-stained RT-PCR products of mice from uninjected (-, no ASO control), or 300 ag of Cep290 (negative control ASO; Gerard et al, Mol. Ther. Nuc. Ac., 2015), ASO-injected brains. Products corresponding to NMD exon inclusion and full-length can be quantified and the percentage of NMD exon inclusion can be plotted. TaqMan PCR can be performed using two different probes spanning NMD exon junctions and the products can be normalized to GAPDH internal control, and fold-change of ASO-injected relative to Cep290-injected brains can be plotted.

### Example 8: Intravenous (IV) Injection of Selected ASOs

PAGEs of SYBR safe-stained RT-PCR products of mice from uninjected (-, no ASO control), or 300 g of Cep290 (negative control ASO; Gerard et al, Mol. Ther. Nuc. Ac., 2015), ASO-injected brains. Products corresponding to NMD exon inclusion and full-length can be quantified and the percentage of NMD exon inclusion can be plotted. TaqMan PCR can be performed using two different probes spanning NMD exon junctions and the products can be normalized to GAPDH internal control and fold-change of ASO-injected relative to Cep290-injected brains can be plotted.

### Example 9: Effect on RNA Splicing and Protein Expression by Treatment with Selected ASOs

In one experiment, ReNcell VM cells were transfected with different exemplary ASOs according to some embodiments of the present disclosure, non-targeting ASO control (“NTC”), or no ASO (“mock control”), in the absence of cycloheximide. RNA was isolated 24 hours after transfection and analyzed for impact on SETD5 mRNA splicing and SETD5 mRNA expression. Briefly, TaqMan qPCR reactions using probes that span Exons 5 and 6 junction (based on the transcript NM_001080517) were conducted to measure productive SETD5 mRNA; TaqMan qPCR reactions using probes that span across NMX Exon 5X only were conducted to measure non-productive SETD5 mRNA that contain Exon 5X and that would be degraded by NMD; TaqMan qPCR reactions using probes that span the Exon 13 and 14 junction (based on the transcript NM_001080517) were conducted to measure total SETD5 mRNA level, including both productive and non-productive SETD5 mRNAs.

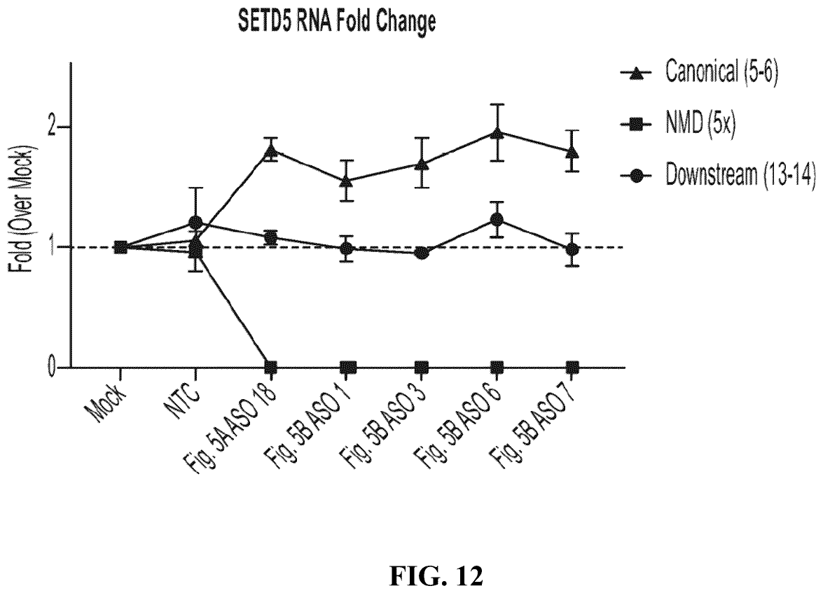

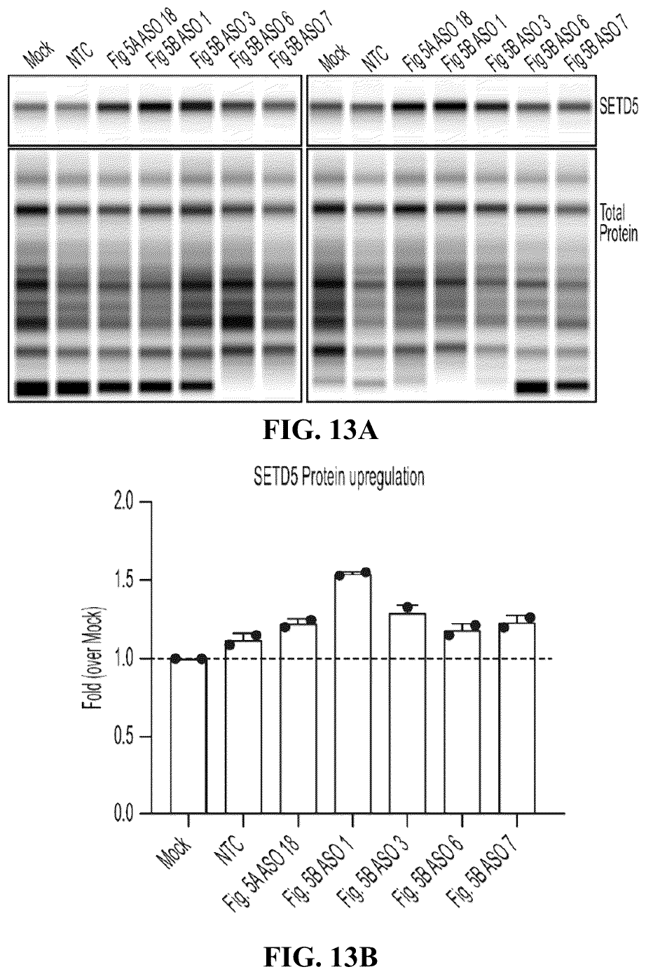

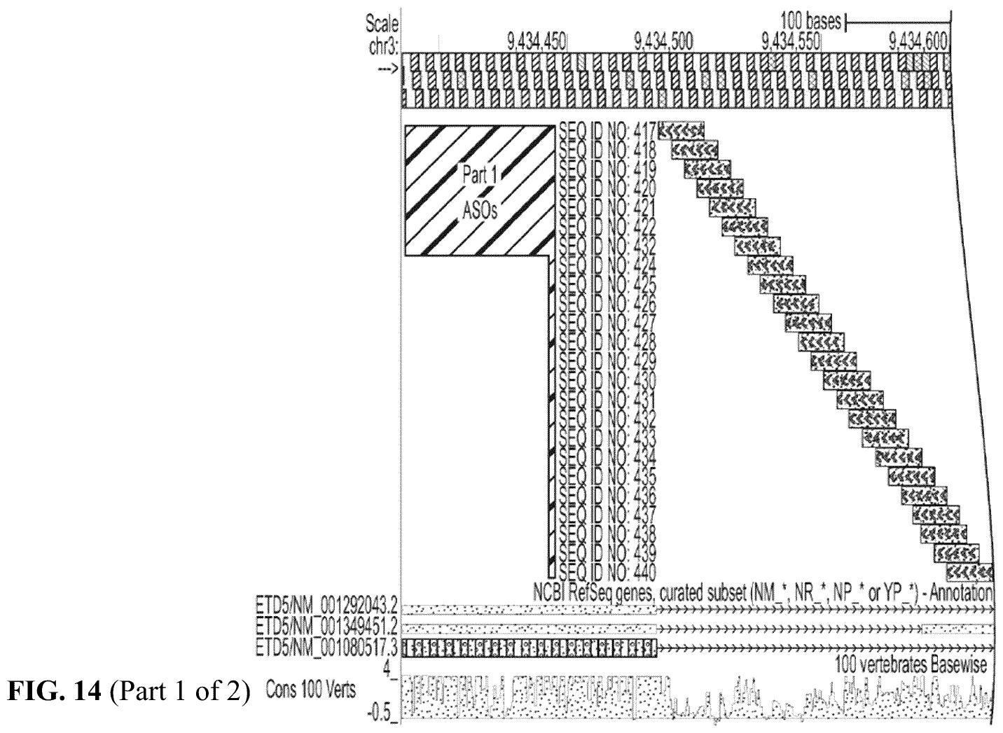

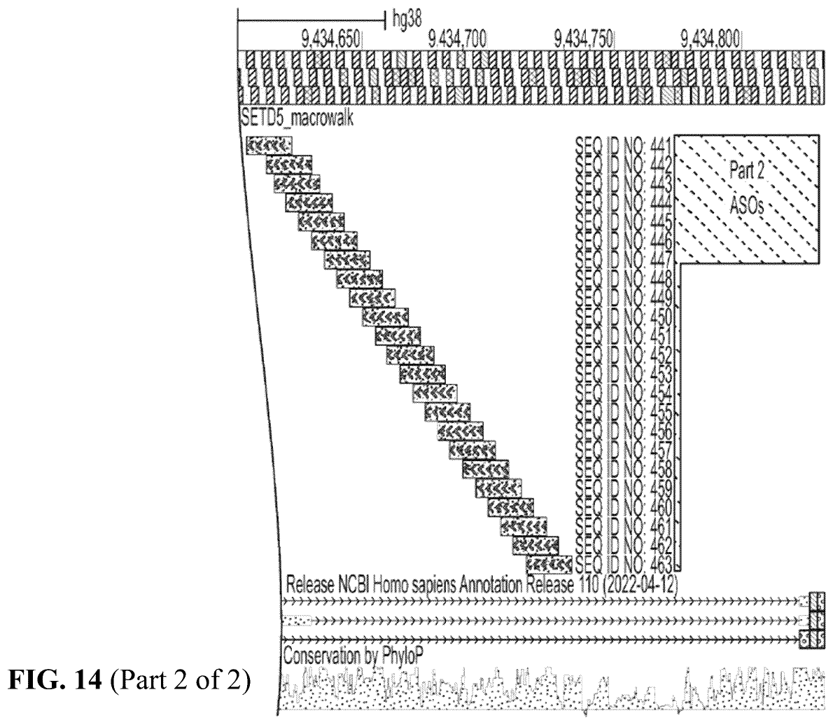

FIG. 12 is a plot that summarizes the relative fold changes in the levels of different SETD5 RNA transcripts in response to the ASO treatment. As shown in the figure, all exemplary ASOs that were tested, including ASO 18 shown in FIG. 5A, and ASO 1, 3, 6, and 7 shown in FIG. 5B, were shown to increase productive SETD5 mRNA transcripts as measured by probes spanning the Exon 5 and 6 junction (“Canonical (5-6)”), as compared to both mock control and NTC, while reducing the level of non-productive SETD5 mRNA transcripts down to almost zero, as measured by probes spanning NMD Exon 5X (“NMD (5X)”). The tested ASOs did not change the total level of SETD5 mRNA transcripts. FIGS. 13A and 13B show SETD5 protein expression level change in response to ASO treatment, as measured by Jess Western blotting. Briefly, ReNcell VM cells were transfected with different exemplary ASOs according to some embodiments of the present disclosure, non-targeting ASO control (“NTC”), or no ASO (“mock control”), in the absence of cycloheximide. 72 hours after transfection the cells were lysed, and the protein lysates were analyzed for impact on SETD5 protein expression by Jess Western blotting. FIG. 13A shows a representative Jess Western blot image of SETD5 protein the different experiment conditions, together with an image of total protein level as a loading control. FIG. 13B is a plot summarizing the fold change in SETD5 protein level as assessed by Jess Western blot and normalized by the total protein level. As shown in the figure, all tested ASOs were shown to increase the protein level of SETD5 in the cells.

### Example 10: Effects on Target Engagement and Gene Expression in HEK293 Cells by Treatment with Selected Exemplary ASOs at Different Concentrations

HEK293 cells were nucleofected with or allowed to freely uptake different exemplary ASO compounds that are listed in Table 6 according to some embodiments of the present disclosure. The cells were then evaluated at fixed time points (24 hours or 3 days post ASO treatment) to assess the fold difference in the level of target engagement and gene expression by the tested ASOs relative to the mock control or SMN control used in the same conditions. Test ASOs included those that overlapped with splice sites (splice-site ASOs), introns (intronic ASOs), and exons (exonic ASOs) in the SETD5 macrowalk and SETD5 microwalks. All cytosines in tested exemplary ASOs were methylcytosines.

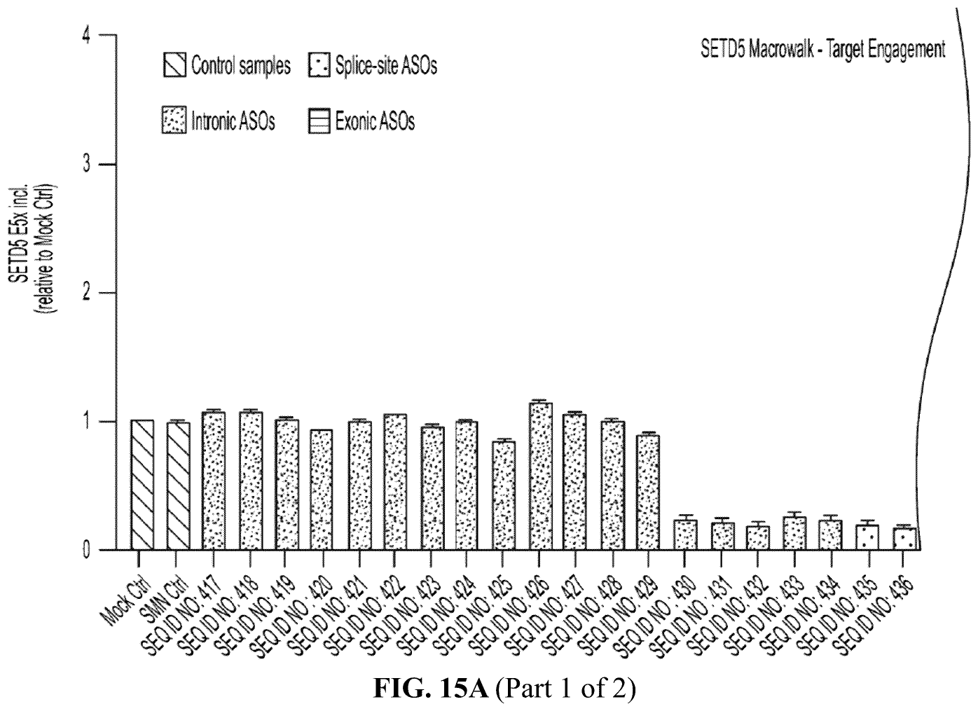

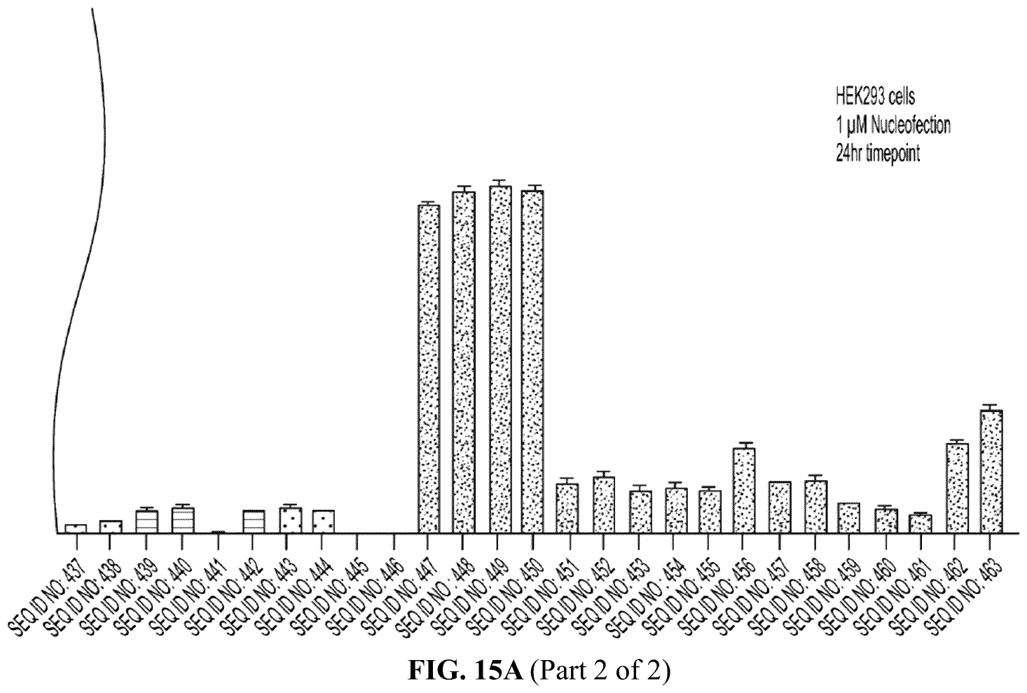

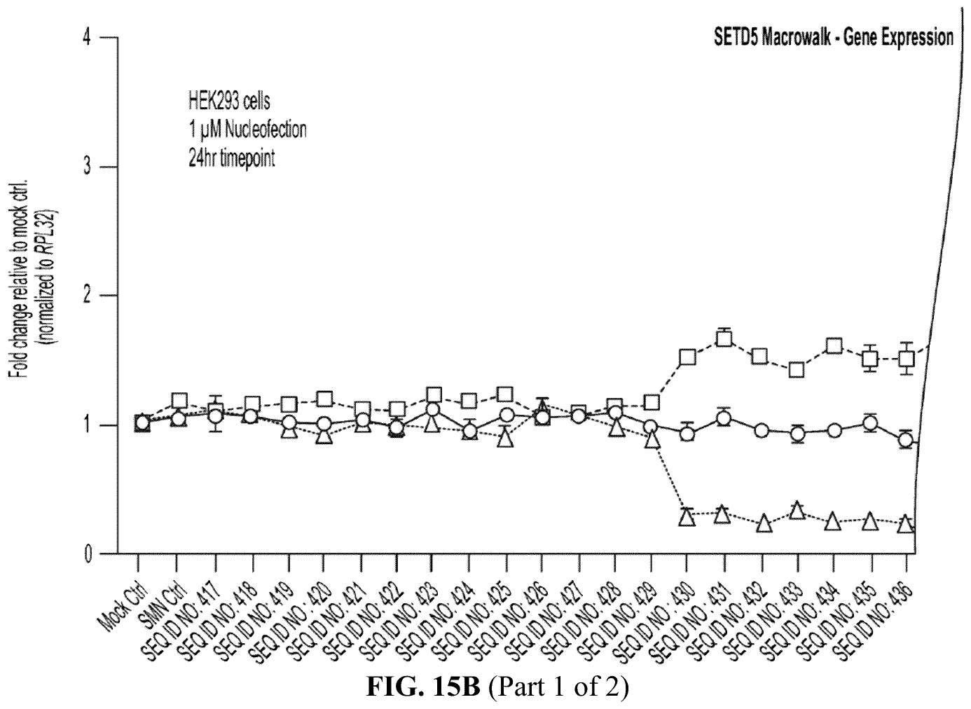

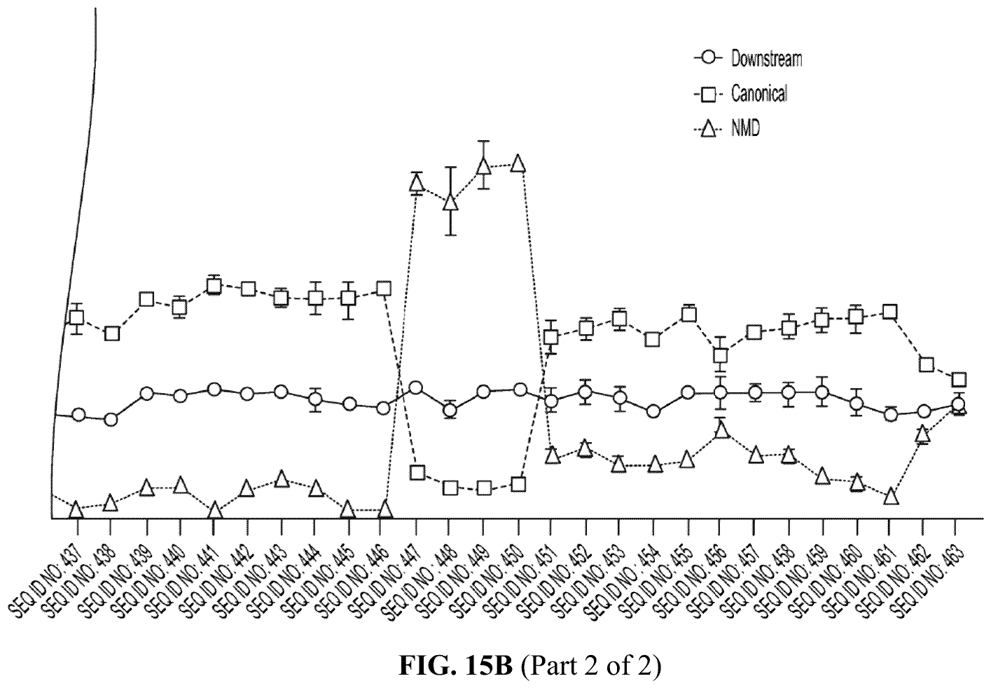

In one such experiment, HEK293 cells were nucleofected with 1 μM of test ASOs and evaluated 24 hours after nucleofection (FIGS. 15A-15B). SETD5 Exon 5X inclusion percentage and SETD5 gene expression (transcript levels) relative to the mock control are summarized in FIGS. 15A-15B, respectively. “NMD” transcript levels in FIG. 15B indicate the levels of SETD5 transcripts containing NMD Exon 5X, “canonical” transcript levels indicate the levels of SETD5 transcripts that do not include NMD Exon 5X, and “downstream” transcript levels indicate the total level of SETD5 transcripts measured by RT-PCR with probes covering mRNA regions downstream of Exon 5X.

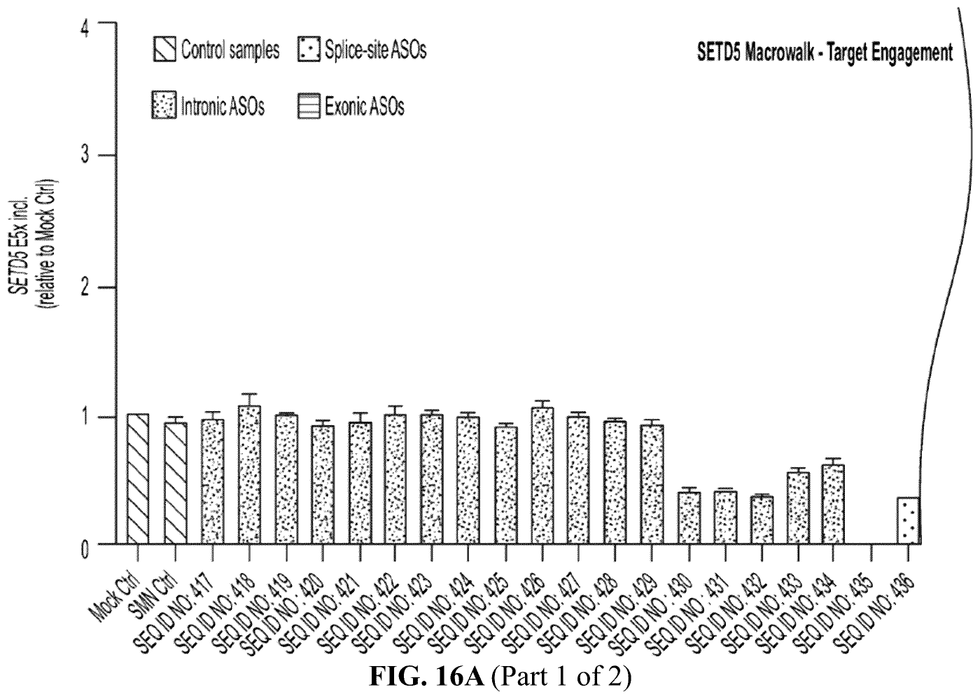

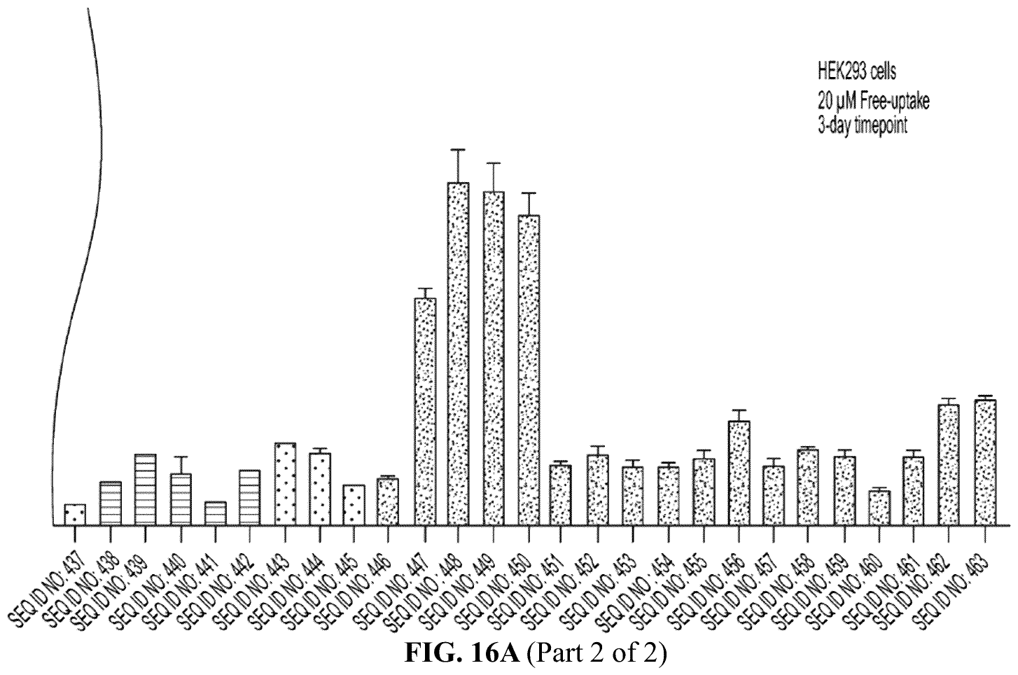

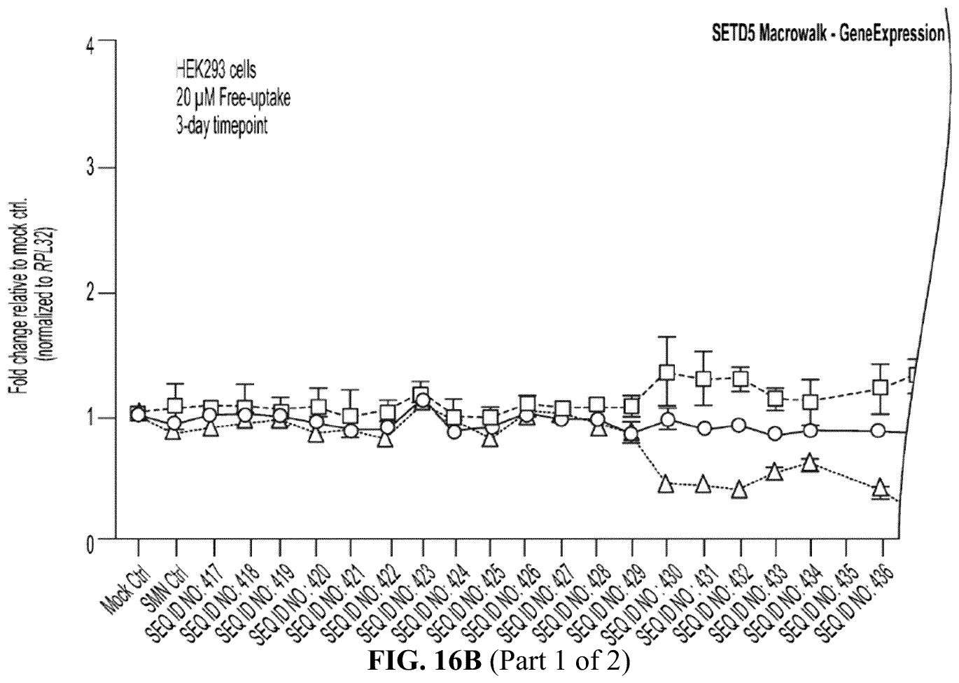

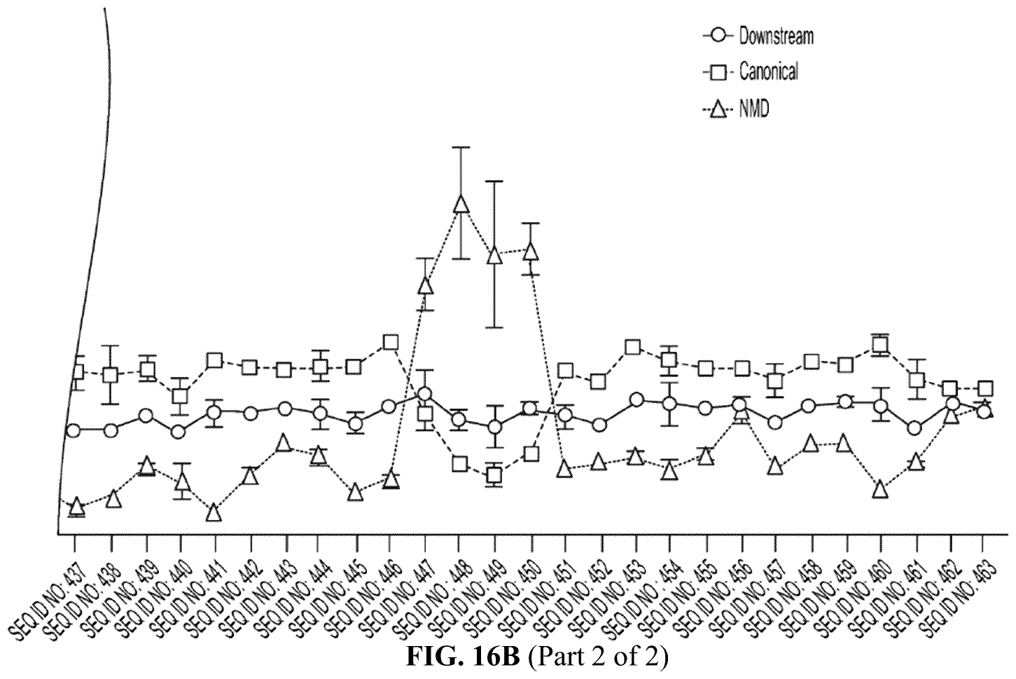

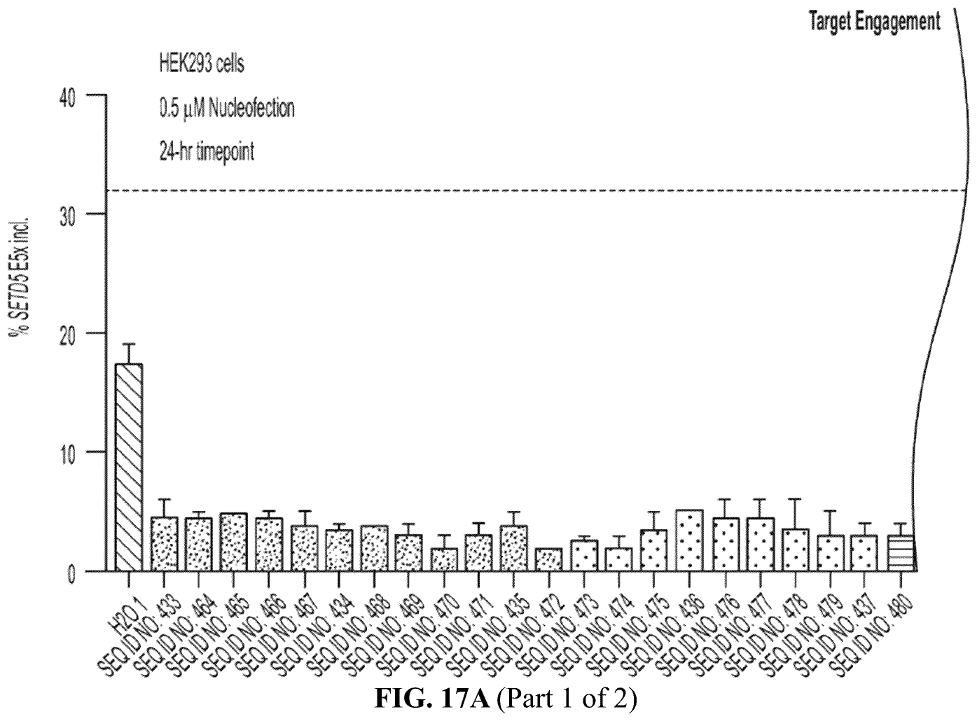

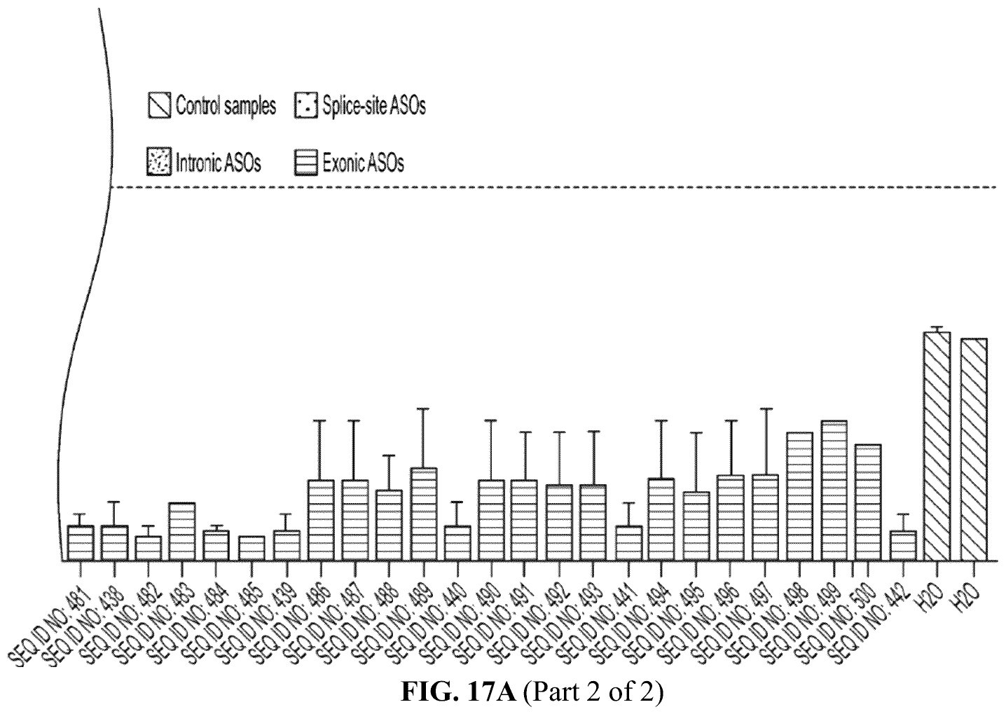

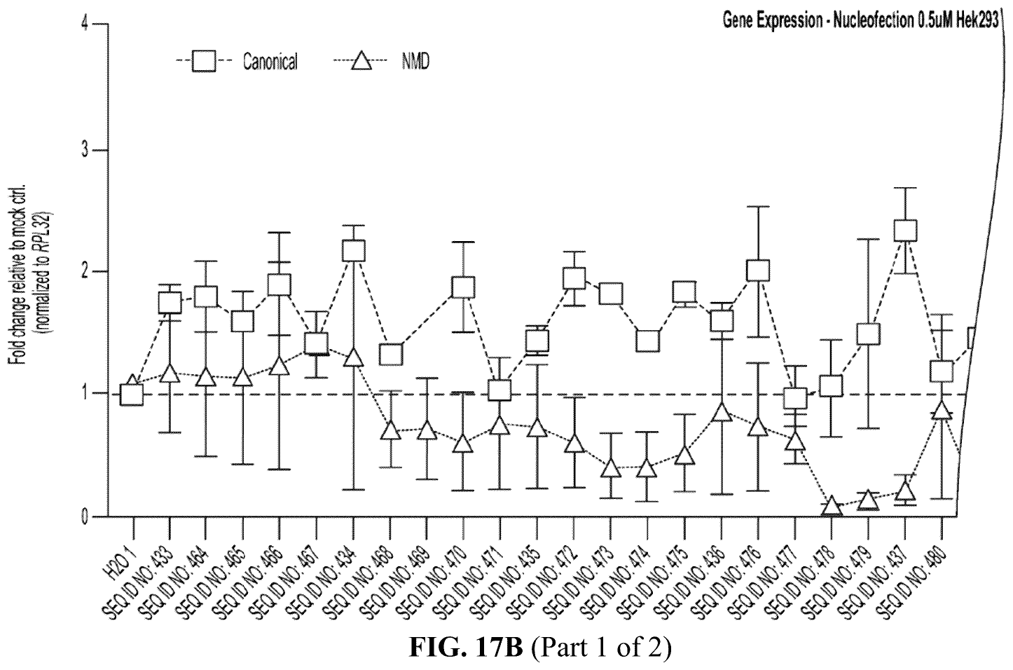

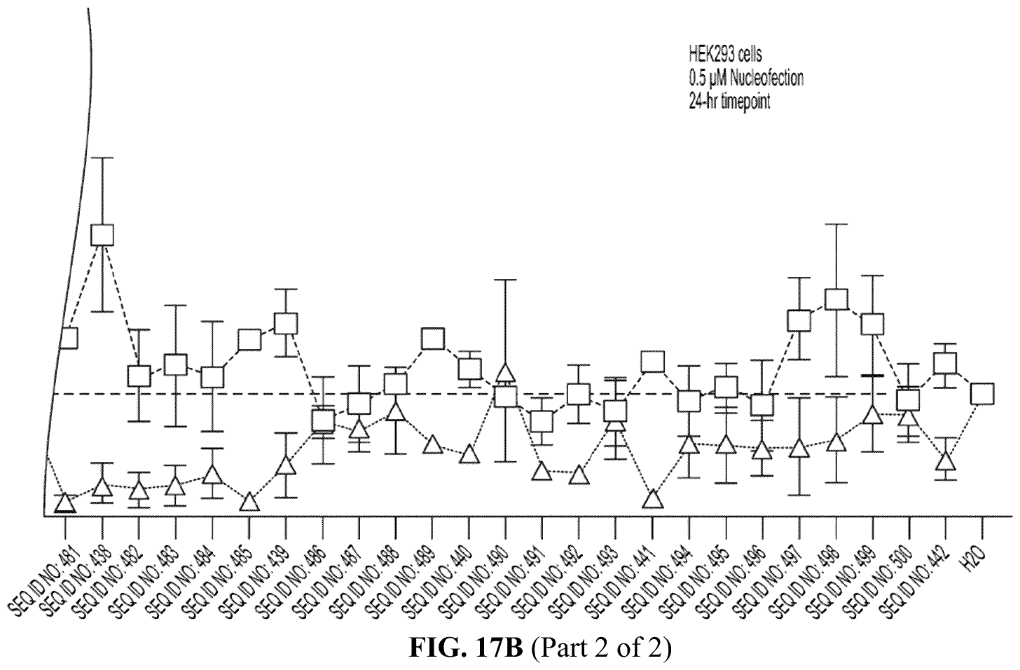

In another experiment, HEK293 cells were allowed to freely uptake 20 μM of test ASOs and evaluated 3 days after the initiation of ASO exposure (FIGS. 16A-16B). SETD5 Exon 5X inclusion percentage and SETD5 gene expression (transcript levels) relative to the mock control are summarized in FIGS. 16A-16B, respectively. In a similar experiment, HEK293 cells were nucleofected with 0.5 μM of test ASOs and evaluated 24 hours after nucleofection (FIGS. 17A-17B). SETD5 Exon 5X inclusion percentage and SETD5 gene expression (transcript levels) relative to the mock control are summarized in FIGS. 17A-17B, respectively.

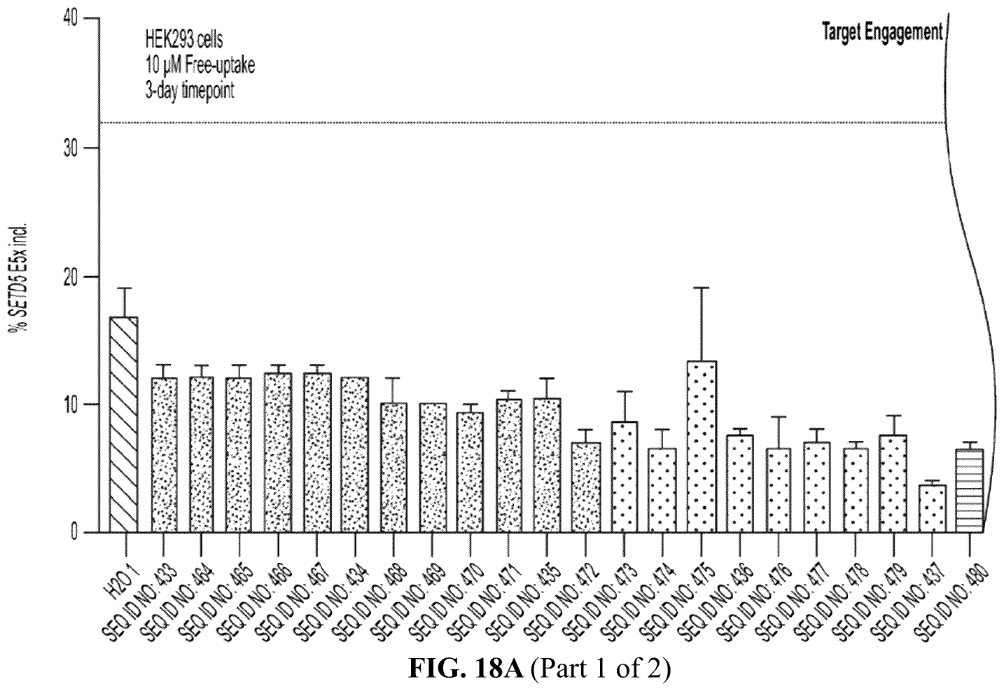

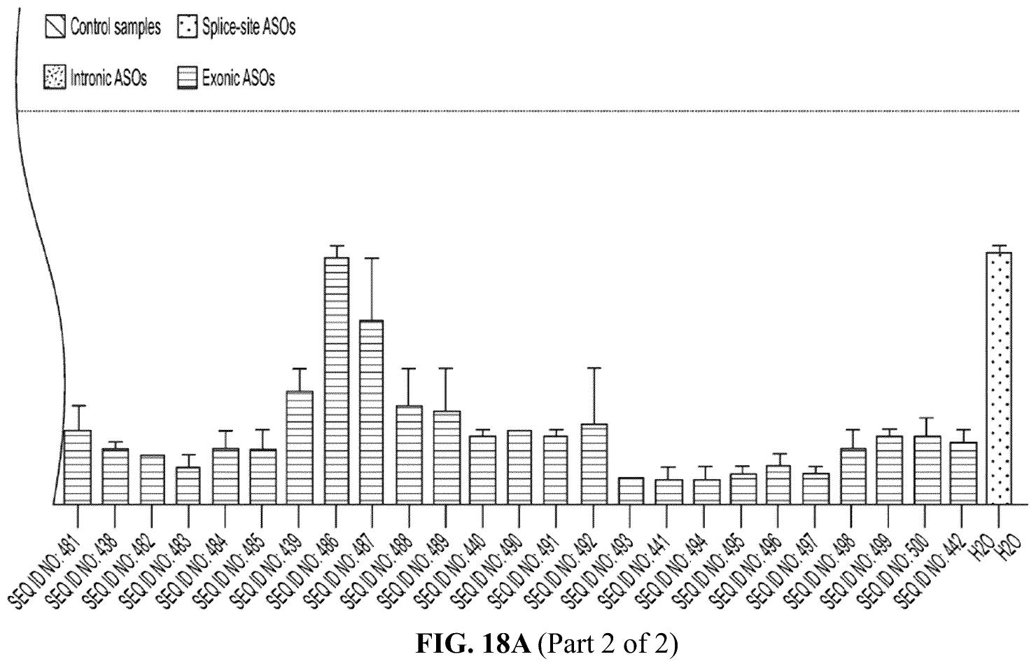

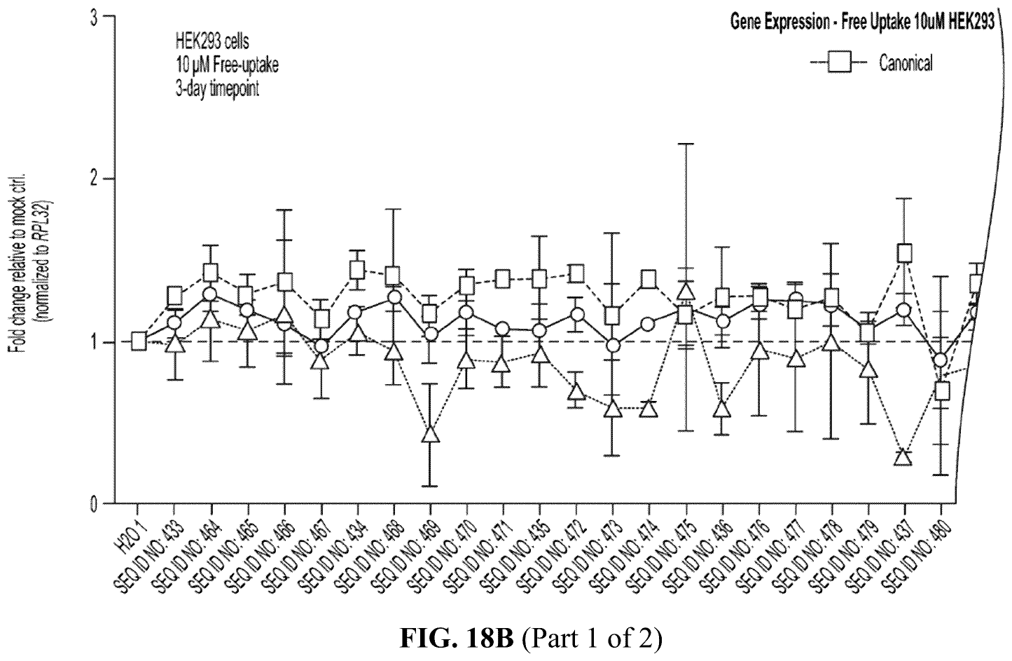

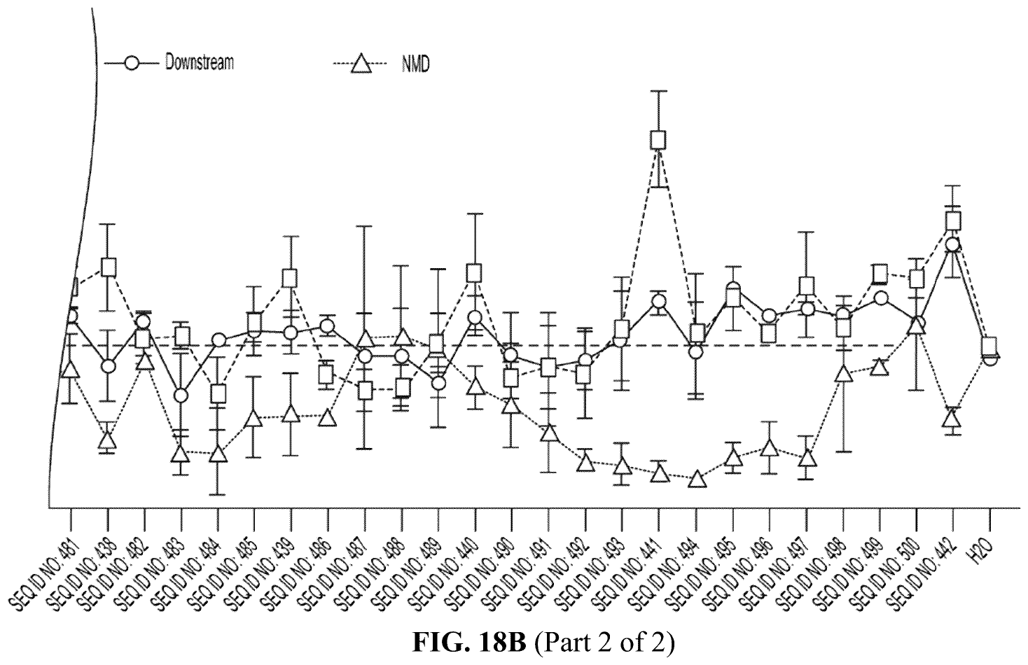

In yet another experiment, HEK293 cells were allowed to freely uptake test ASOs at 10 μM and evaluated 3 days after nucleofection (FIGS. 18A-18B). SETD5 Exon 5X inclusion percentage and SETD5 gene expression (transcript levels) relative to the mock control are summarized in FIGS. 17A-17B, respectively.

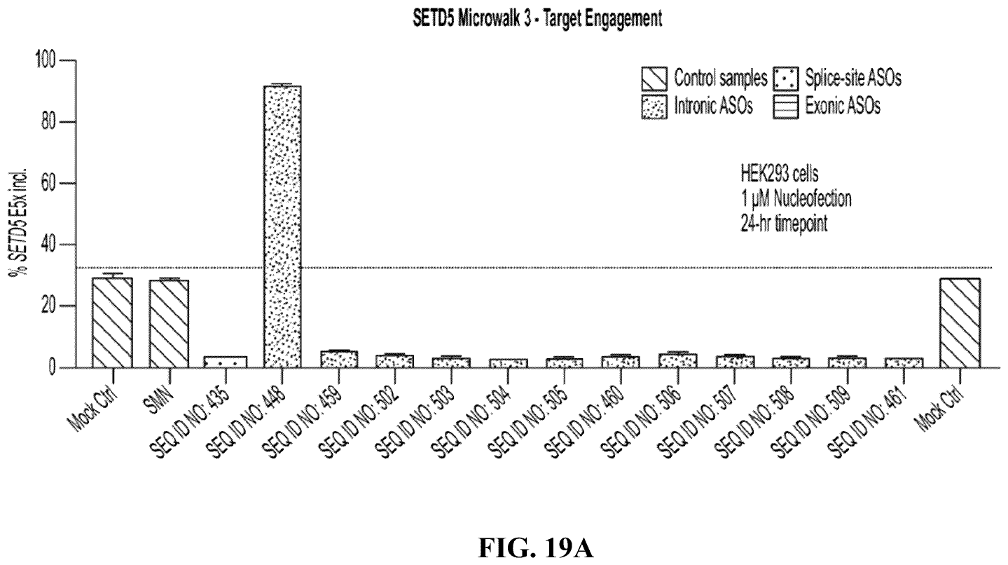

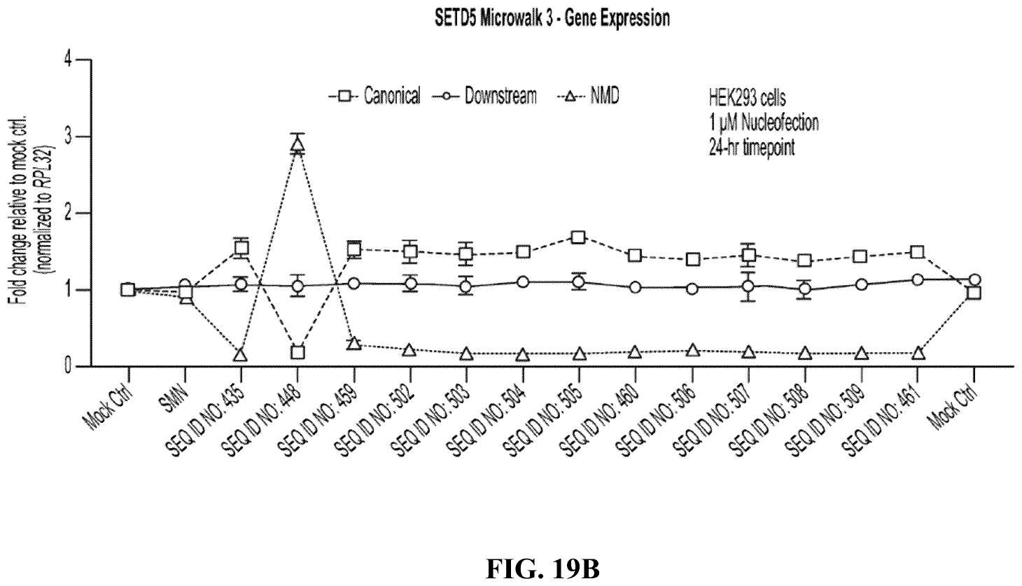

In another experiment, a subset of intronic ASOs generated from an SETD5 microwalk were treated under the same conditions (1 μM of test ASOs and evaluated 24 hours after nucleofection) and the percentage of SETD5 Exon 5X inclusion (FIG. 19A) and fold change in gene expression were assessed (FIG. 19B). FIG. 19A shows a histogram representing the percentage of SETD5 Exon 5X inclusion in HEK293 cells post application of various ASOs relative to mock and SMN-targeting (SMN1 survival of motor neuron 1-targeting) ASO controls. FIG. 19B is a graph representing the fold change of gene expression by various ASOs from the SETD5 microwalk relative to control samples.

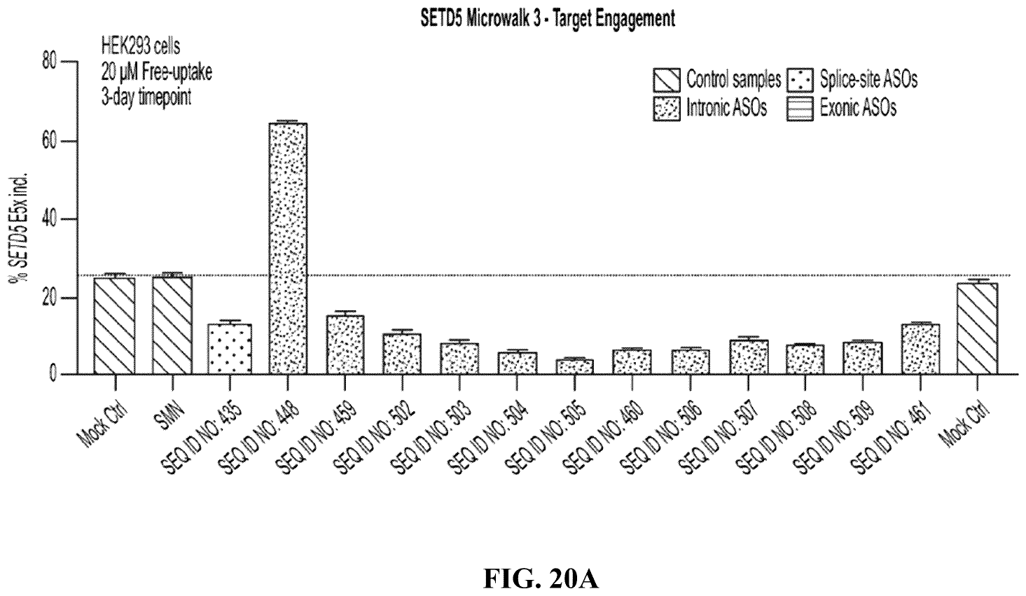

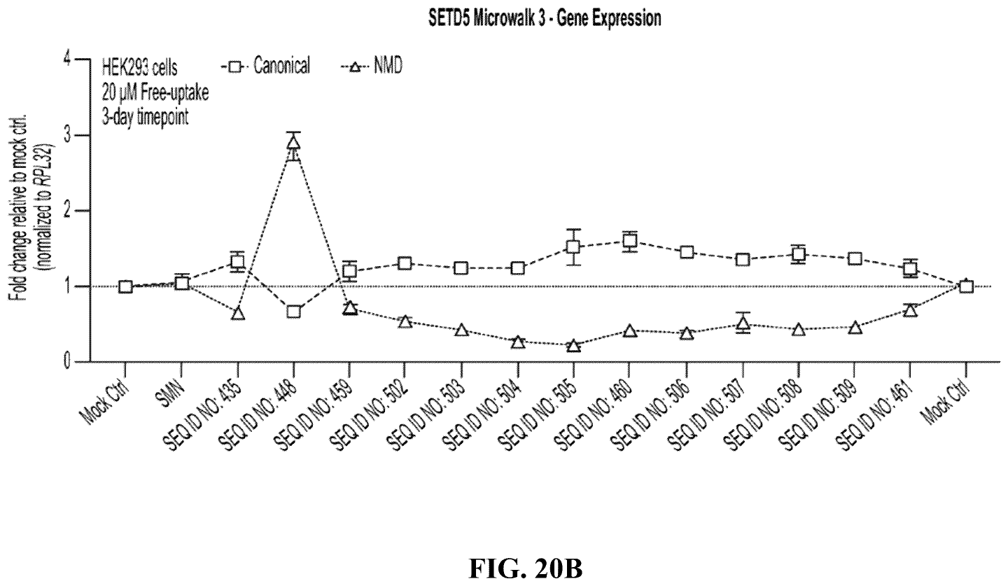

In yet another experiment, a subset of intronic ASOs generated from an SETD5 microwalk were treated under the same conditions (20 μM of test ASOs and evaluated 3 days after the initiation of ASO exposure) and the percentage of SETD5 Exon 5X inclusion (FIG. 20A) and fold change in gene expression were assessed (FIG. 20B). FIG. 20A shows a histogram representing the percentage of SETD5 Exon 5X inclusion in HEK293 cells post application of various ASOs relative to mock and SMN controls. FIG. 20B is a graph representing the fold change of gene expression by various ASOs from the SETD5 microwalk relative to control samples.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXEMPLARY EMBODIMENTS

Described herein, in certain embodiments, is a method of modulating expression of a target protein in a cell having a pre-mRNA that is transcribed from a target gene and that comprises a non-sense mediated RNA decay-inducing exon (NMD exon), the method comprising contacting an agent or a vector encoding the agent to the cell, whereby the agent modulates splicing of the NMD exon from the pre-mRNA, thereby modulating the level of a processed mRNA that is processed from the pre-mRNA, and modulating the expression of the target protein in the cell, wherein the target gene is a SETD5 gene.

Described herein, in certain embodiments, is a method of treating or reducing the likelihood of developing a disease or condition in a subject in need thereof by modulating expression of a target protein in a cell of the subject, comprising: contacting the cell of the subject with an agent or a vector encoding the agent to the cell, whereby the agent modulates splicing of a non-sense mediated mRNA decay-inducing exon (NMD exon) from a pre-mRNA that is transcribed from a target gene and that comprises the NMD exon, thereby modulating the level of a processed mRNA that is processed from the pre-mRNA, and modulating the expression of the target protein in the cell of the subject, wherein the gene is a SETD5 gene.

In some embodiments, the agent: (a) binds to a targeted portion of the pre-mRNA; (b) modulates binding of a factor involved in splicing of the NMD exon; or (c) a combination of (a) and (b).

In some embodiments, the agent interferes with binding of the factor involved in splicing of the NMD exon to a region of the targeted portion.

In some embodiments, the targeted portion of the pre-mRNA is proximal to the NMD exon.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NMD exon.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NMD exon.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NMD exon.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NMD exon.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site selected from the group consisting of: GRCh38/hg38: chr3 9429827; GRCh38/hg38: chr3 9433370; GRCh38/hg38: chr3 9434591 and GRCh38/hg38: chr3 9428823 9429009.

In some embodiments, the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site selected from the group consisting of: GRCh38/hg38: chr3 9429827; GRCh38/hg38: chr3 9433370; GRCh38/hg38: chr3 9434591 and GRCh38/hg38: chr3 9428823 9429009.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr3 9430051; GRCh38/hg38: chr3 9433562; GRCh38/hg38: chr3 9434630 and GRCh38/hg38: chr3 9428823 9429009.

In some embodiments, the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr3 9430051; GRCh38/hg38: chr3 9433562; GRCh38/hg38: chr3 9434630 and GRCh38/hg38: chr3 9428823 9429009.

In some embodiments, the targeted portion of the pre-mRNA is located in an intronic region between two canonical exonic regions of the pre-mRNA, and wherein the intronic region contains the NMD exon.

In some embodiments, the targeted portion of the pre-mRNA at least partially overlaps with the NMD exon.

In some embodiments, targeted portion of the pre-mRNA at least partially overlaps with an intron upstream or downstream of the NMD exon.

In some embodiments, the targeted portion of the pre-mRNA comprises 5′ NMD exon-intron junction or 3′ NMD exon-intron junction.

In some embodiments, the targeted portion of the pre-mRNA is within the NMD exon.

In some embodiments, the targeted portion of the pre-mRNA comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

In some embodiments, the NMD exon comprises a sequence with at least 80%, at least 90%, or 100% sequence identity to a sequence selected from the group consisting of the sequences listed in Table 2 (e.g., the sequence set forth in any one of SEQ ID NO: 332-339).

In some embodiments, the NMD exon comprises a sequence selected from the group consisting of the sequences listed in Table 2 (e.g., the sequence set forth in any one of SEQ ID NO: 332-339).

In some embodiments, the pre-mRNA comprises a sequence with at least about 80%, 85%, 90%, 95%, 97%, or 100% sequence identity to a sequence selected from the group consisting of the sequences listed in Table 2 or Table 3 (e.g., the sequence set forth in any one of SEQ ID NO: 332-403).

In some embodiments, the pre-mRNA is encoded by a genetic sequence with at least about 80%, 85%, 90%, 95%, 97%, or 100% sequence identity to a sequence selected from the group consisting of the sequences listed in Table 2 or Table 3 (e.g., the sequence set forth in any one of SEQ ID NO: 332-403).

In some embodiments, the targeted portion of the pre-mRNA comprises a sequence with at least 80%, 85%, 90%, 95%, 97%, or 100% sequence identity to a region comprising at least 8 contiguous nucleic acids of a sequence selected from the group consisting of the sequences listed in Table 2 or Table 3 (e.g., the sequence set forth in any one of SEQ ID NO: 332-403).

In some embodiments, the agent is an antisense oligomer (ASO) and wherein the ASO comprises a sequence that is at least about 80%, 85%, 90%, 95%, 97%, or 100% complementary to at least 8 contiguous nucleic acids of a sequence selected from the group consisting of the sequences listed in Table 4 (e.g., the sequence set forth in any one of SEQ ID NO: 1-331).

In some embodiments, the method comprises contacting the vector encoding the agent to the cell, wherein the agent is a polynucleotide comprising an antisense oligomer.

In some embodiments, the vector is a viral vector.

In some embodiments, the viral vector is an adeno-associated viral vector.

In some embodiments, the polynucleotide further comprises a modified snRNA.

In some embodiments, the modified human snRNA is a modified U1 snRNA or a modified U7 snRNA.

In some embodiments, the modified human snRNA is a modified U7 snRNA and wherein the antisense oligomer has a sequence at least 75%, at least 80%, at least 85%, at least 90%, at least 95%, at least 98%, at least 99%, or 100% identical to a sequence listed in Table 4 (e.g., the sequence set forth in any one of SEQ ID NO: 1-331).

In some embodiments, the targeted portion of the pre-mRNA is within the non-sense mediated RNA decay-inducing exon selected from the group consisting of: GRCh38/hg38: chr3 9429827 9430051; GRCh38/hg38: chr3 9433370 9433562; GRCh38/hg38: chr3 9434591 9434630 and GRCh38/hg38: chr3 9428823 9429009.

In some embodiments, the targeted portion of the pre-mRNA is upstream or downstream of the non-sense mediated RNA decay-inducing exon selected from the group consisting of: GRCh38/hg38: chr3 9429827 9430051; GRCh38/hg38: chr3 9433370 9433562; GRCh38/hg38: chr3 9434591 9434630 and GRCh38/hg38: chr3 9428823 9429009.

In some embodiments, the targeted portion of the pre-mRNA comprises an exon-intron junction of exon selected from the group consisting of: GRCh38/hg38: chr3 9429827 9430051; GRCh38/hg38: chr3 9433370 9433562; GRCh38/hg38: chr3 9434591 9434630 and GRCh38/hg38: chr3 9428823 9429009.

In some embodiments, the target protein expressed from the processed mRNA is a full-length protein or a wild-type protein.

In some embodiments, the target protein expressed from the processed mRNA is at least partially functional as compared to a wild-type SETD5 protein.

In some embodiments, the target protein expressed from the processed mRNA is at least partially functional as compared to a full-length wild-type SETD5 protein.

In some embodiments, the agent promotes exclusion of the NMD exon from the processed mRNA.

In some embodiments, the exclusion of the NMD exon from the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA in a control cell.

In some embodiments, the agent increases the level of the processed mRNA in the cell.

In some embodiments, the level of the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA in a control cell.

In some embodiments, the agent increases the expression of the target protein in the cell.

In some embodiments, a level of the target protein expressed from the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein produced in a control cell.

In some embodiments, the disease or condition is induced by a loss-of-function mutation in the target protein.

In some embodiments, the disease or condition is associated with haploinsufficiency of a gene encoding the target protein, and wherein the subject has a first allele encoding a functional target protein, and a second allele from which the target protein is not produced or produced at a reduced level, or a second allele encoding a nonfunctional target protein or a partially functional target protein.

In some embodiments, the disease or condition is an intellectual disability or an autism spectrum disease.

In some embodiments, the disease or condition is selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21).

In some embodiments, the disease or condition is associated with an autosomal recessive mutation of a gene encoding the target protein, wherein the subject has a first allele encoding from which: (i) the target protein is not produced or produced at a reduced level compared to a wild-type allele; or (ii) the target protein produced is nonfunctional or partially functional compared to a wild-type allele, and a second allele from which: (iii) the target protein is produced at a reduced level compared to a wild-type allele and the target protein produced is at least partially functional compared to a wild-type allele; or (iv) the target protein produced is partially functional compared to a wild-type allele.

In some embodiments, the disease or condition is an intellectual disability or an autism spectrum disease.

In some embodiments, the disease or condition is selected from the group consisting of: autism; mental retardation; mental retardation, autosomal dominant 23 (mrd23, autosomal dominant non-syndromic intellectual disability 23; autosomal dominant mental retardation 23; intellectual disability-facial dysmorphism syndrome due to setd5 haploinsufficiency; mental retardation, autosomal dominant, type 23); Cornelia De Lange Syndrome (cdls, typus degenerativus amstelodamensis, de lange syndrome, brachmann-de lange syndrome, bdls, brachmann de lange syndrome); alacrima, achalasia, and mental retardation syndrome (aamr, intellectual disability); chromosome 3pter-p25 deletion syndrome (3p-syndrome; 3p deletion syndrome; distal monosomy 3p; chromosome 3p-syndrome; chromosome 3, monosomy 3p25; del syndrome; deletion 3p25; distal 3p deletion; monosomy 3pter; telomeric monosomy 3p; partial monosomy 3p; 3p partial monosomy syndrome; chromo-some 3, deletion 3p; chromosome 3, monosomy 3p; chromosome 3p deletion syndrome; deletion 3p; monosomy 3p; deletion syndrome, chromosome 3pter-p25NK-cell enteropathy); and mental retardation, autosomal dominant 21 (mrd21; autosomal dominant non-syndromic intellectual disability 21; autosomal dominant mental retardation 21; intellectual disability-feeding difficulties-developmental delay-microcephaly syndrome; mental retardation, autosomal dominant, type 21).

In some embodiments, the agent promotes exclusion of the NMD exon from the processed mRNA and increases the expression of the target protein in the cell.

In some embodiments, the agent inhibits exclusion of the NMD exon from the processed mRNA encoding the target protein.

In some embodiments, the exclusion of the NMD exon from the processed mRNA in the cell contacted with the agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA in a control cell.

In some embodiments, the agent decreases the level of the processed mRNA in the cell.

In some embodiments, the level of the processed mRNA in the cell contacted with the agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA in a control cell.

In some embodiments, the agent decreases the expression of the target protein in the cell.

In some embodiments, a level of the target protein expressed from the processed mRNA in the cell contacted with the agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein expressed in a control cell.

In some embodiments, the disease or condition is induced by a gain-of-function mutation in the target protein.

In some embodiments, the subject has an allele from which the target protein is produced at an increased level, or an allele encoding a mutant target protein that exhibits increased activity in the cell.

In some embodiments, the agent inhibits exclusion of the NMD exon from the processed mRNA encoding the target protein and decreases the expression of the target protein in the cell.

In some embodiments, the agent is an antisense oligomer (ASO) and wherein the antisense oligomer comprises a backbone modification comprising a phosphorothioate linkage or a phosphorodiamidate linkage.

In some embodiments, the agent is an antisense oligomer (ASO) and wherein the antisense oligomer comprises a phosphorodiamidate morpholino, a locked nucleic acid, a peptide nucleic acid, a 2′-O-methyl, a 2′-Fluoro, a 2′-O-methoxyethyl moiety, or a 2′-NMA moiety.

In some embodiments, the agent is an antisense oligomer (ASO) and wherein the antisense oligomer comprises at least one modified sugar moiety.

In some embodiments, each sugar moiety is a modified sugar moiety.

In some embodiments, the agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some embodiments, the agent is an antisense oligomer (ASO) and wherein the antisense oligomer is at least 80%, at least 85%, at least 90%, at least 95%, at least 98%, at least 99%, or 100%, complementary to the targeted portion of the pre-mRNA.

In some embodiments, the method further comprises assessing mRNA level or expression level of the target protein.

In some embodiments, the subject is a human.

In some embodiments, the subject is a non-human animal.

In some embodiments, the subject is a fetus, an embryo, or a child.

In some embodiments, the cells are ex vivo.

In some embodiments, the agent is administered by intrathecal injection, intracerebroventricular injection, intraperitoneal injection, intramuscular injection, subcutaneous injection, intravitreal, or intravenous injection of the subject.

In some embodiments, the method further comprises administering a second therapeutic agent to the subject.

In some embodiments, the second therapeutic agent is a small molecule.

In some embodiments, the second therapeutic agent is an antisense oligomer.

In some embodiments, the second therapeutic agent corrects intron retention.

In some embodiments, the pre-mRNA comprises one or more NMD exons.

In some embodiments, the pre-mRNA comprises two or more NMD exons.

In some embodiments, the pre-mRNA comprises three or more NMD exons.

In some embodiments, splicing of one or more NMD exons from the pre-mRNA are modulated.

In some embodiments, splicing of two or more NMD exons from the pre-mRNA are modulated.

In some embodiments, splicing of three or more NMD exons from the pre-mRNA are modulated.

In some embodiments, the two or more NMD exons are located in a single intron.

In some embodiments, the two or more NMD exons are located in different introns.

In some embodiments, the three or more NMD exons are located in a single intron.

In some embodiments, the three or more NMD exons are located in different introns.

In some embodiments, the method treats the disease or condition.

Described herein, in certain embodiments, is a composition comprising an agent or a vector encoding the agent that modulates splicing of a non-sense mediated RNA decay-inducing exon (NMD exon) from a pre-mRNA that is transcribed from a target gene and that comprises the NMD exon, thereby modulating the level of a processed mRNA that is processed from the pre-mRNA, and modulating expression of a target protein in a cell having the pre-mRNA, wherein the target gene is a SETD5 gene.

Described herein, in certain embodiments, is a composition comprising an agent or a vector encoding the agent that modulates splicing of a non-sense mediated mRNA decay-inducing exon (NMD exon) from a pre-mRNA that is transcribed from a target gene and that comprises the NMD exon, thereby treating a disease or condition in a subject in need thereof by modulating the level of a processed mRNA that is processed from the pre-mRNA, and modulating expression of a target protein in a cell of the subject, wherein the target gene is a SETD5 gene.

Described herein, in certain embodiments, is a pharmaceutical composition comprising the composition as described herein; and a pharmaceutically acceptable excipient and/or a delivery vehicle.

LENGTHY TABLES
The patent application contains a lengthy table section. A copy of the table is available in electronic form from the USPTO web site (https://seqdata.uspto.gov/docdetail?docId=US20260035694A1). An electronic copy of the table will also be available from the USPTO upon request and payment of the fee set forth in 37 CFR 1.19(b)(3).

1-94. (canceled)

95. A method of modulating expression of a target protein in a cell having a pre-mRNA that is transcribed from a target gene and that comprises a non-sense mediated RNA decay-inducing exon (NMD exon), the method comprising contacting an agent or a vector encoding the agent to the cell, whereby the agent modulates splicing of the NMD exon from the pre-mRNA, thereby modulating the level of a processed mRNA that is processed from the pre-mRNA, and modulating the expression of the target protein in the cell, wherein the target gene is a SETD5 gene.

96. The method of claim 95, wherein the agent:
(a) binds to a targeted portion of the pre-mRNA;
(b) modulates binding of a factor involved in splicing of the NMD exon; or
(c) a combination of (a) and (b).

97. The method of claim 96, wherein the targeted portion of the pre-mRNA:
(i) is proximal to the NMD exon;
(ii) is located in an intronic region between two canonical exonic regions of the pre-mRNA, and wherein the intronic region contains the NMD exon;
(iii) at least partially overlaps with the NMD exon;
(iv) at least partially overlaps with an intron upstream or downstream of the NMD exon;
(v) comprises a 5′ NMD exon-intron junction or a 3′ NMD exon-intron junction;
(vi) is within the NMD exon; or
(vii) comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

98. The method of claim 96, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr3 9429827; GRCh38/hg38: chr3 9433370; GRCh38/hg38: chr3 9434591; and GRCh38/hg38: chr3 9428823.

99. The method of claim 96, wherein the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, or about 50 nucleotides downstream of genomic site selected from the group consisting of: GRCh38/hg38: chr3 9430051; GRCh38/hg38: chr3 9433562; GRCh38/hg38: chr3 9434630 and GRCh38/hg38: chr3 9429009.

100. The method of claim 95, wherein the NMD exon comprises a sequence with at least 90% sequence identity to the sequence set forth in any one of SEQ ID NOs: 333, 332, or 334-339.

101. The method of claim 95, wherein the agent is an antisense oligomer (ASO).

102. The method of claim 101, wherein the ASO comprises a sequence that has at least about 95% sequence identity to at least 8, 9, 10, 11, 12, 13, 14, 15, 16, 17, or 18 contiguous nucleic acids of the sequence set forth in any one of SEQ ID NOs: 1-331 or 417-509.

103. The method of claim 101, wherein the ASO is from 8 to 50 nucleobases in length.

104. The method of claim 95, wherein the method comprises contacting the vector encoding the agent to the cell, wherein the agent is a polynucleotide comprising an antisense oligomer.

105. The method of claim 104, wherein the vector is a viral vector.

106. The method of claim 104, wherein the polynucleotide further comprises a modified snRNA.

107. The method of claim 95, wherein the target protein expressed from the processed mRNA is at least partially functional as compared to a wild-type SETD5 protein.

108. The method of claim 95, wherein the pre-mRNA comprises two or more NMD exons.

109. The method of claim 108, wherein the two or more NMD exons are located in a single intron.

110. The method of claim 108, wherein the two or more NMD exons are located in different introns.

111. The method of claim 95, wherein the agent promotes exclusions of one or more NMD exons from the pre-mRNA, thereby increasing the expression of SETD5 in the cell.

112. A method of modulating expression of a target protein in a cell having a pre-mRNA that is transcribed from a target gene and that comprises a first exon that comprises a translational start site, the method comprising contacting an agent or a vector encoding the agent to the cell, whereby the agent modulates splicing of the first exon that comprises a translational start site from the pre-mRNA, thereby modulating the level of a processed mRNA that is processed from the pre-mRNA, and modulating the expression of the target protein in the cell, wherein the target gene is a SETD5 gene.

113. The method of claim 112, wherein the first exon that comprises a translational start site exon is upstream of a second exon that comprises a second translational start site.

114. The method of claim 112, wherein the first exon that comprises a translational start site is upstream of an NMD exon.
